(19) United States
(12) Patent Application Publication
(10) Pub. No.: US 2018/0192952 A1
(43) Pub. Date: Jul. 12, 2018

(54) FULLY IMPLANTABLE SOFT MEDICAL DEVICES FOR INTERFACING WITH BIOLOGICAL TISSUE

(71) Applicants: The Board of Trustees of the University of Illinois, Urbana (IL); Washington University in St. Louis, St. Louis, MO (US)

(72) Inventors: John A. ROGERS, Wilmette, IL (US); Gunchul SHIN, Urbana, IL (US); Sung Il PARK, Urbana, IL (US); Robert GEREAU, High Ridge, MO (US); Clinton MORGAN, St. Louis, MO (US); Daniel BRENNER, St. Louis, MO (US); Michael BRUCHAS, St. Louis, MO (US); Jordan McCALL, St. Louis, MO (US)

(21) Appl. No.: 15/741,081

(22) PCT Filed: Jul. 1, 2016
(86) PCT No.: PCT/US16/40717

§ 371 Date: Dec. 29, 2017

Related U.S. Application Data
(60) Provisional application No. 62/188,334, filed on Jul. 2, 2015.

(51) Int. Cl. A61B 5/00; A61B 5/01; A61B 5/04; A61B 5/07; A61B 5/03; A61F 7/12; A61N 1/05; A61N 1/372; A61M 31/00; A61N 1/378; A61N 5/06; A61N 1/375

(52) U.S. Cl. CPC A61B 5/686; A61N 2005/0652; A61B 5/01; A61B 5/04; A61B 5/076; A61B 5/4839; A61B 5/036; A61F 7/12; A61N 1/05; A61N 1/37229; A61M 31/002; A61N 1/3787; A61N 5/0601; A61N 1/37518; A61B 2562/164; A61F 2007/126; A61M 2205/3515; A61M 2205/3523; A61M 2205/8243; A61N 2005/0612; A61B 5/0084

(57) ABSTRACT

Provided are fully implantable soft medical devices and related methods. The devices comprise stretchable electronic devices between on an elastomeric substrate and an elastomeric super-state. Electronic components of the electronic device are configured to interface with tissue. Wireless power and control systems provide wireless power and control of the electronic components, thereby providing the fully implantable functionality. The devices may have a plurality of independently addressable electronic components, such as LEDs. In this manner, wireless control of a single implanted device may still provide multi-functional capabilities, including in a multiplexed configuration.

### CROSS-REFERENCE TO RELATED APPLICATIONS

This application claims the benefit of U.S. Provisional Patent Application No. 62/188,334 filed Jul. 2, 2015, which is hereby incorporated by reference to the extent not inconsistent herewith.

### STATEMENT REGARDING FEDERALLY SPONSORED RESEARCH OR DEVELOPMENT

This invention was made with government support under NS081707 awarded by the National Institutes of Health and RO1DA037152 and R21DA035144 awarded by the National Institute on Drug Abuse. The government has certain rights in the invention.

### BACKGROUND OF INVENTION

The devices and methods are in the field of fully implantable soft medical devices without any physical connection to externally positioned components. The ability to wirelessly control and communicate with a fully implanted device that has no observable components external to the body provides a number of important functional benefits.

Conventional implants, in contrast, generally require hard physical connection to a component, such as a light source with fiber optic cable or for effective communication and control via an externally mounted controller or connector thereto. Even implants that are partially implanted, tend to suffer from limitations associated with singleplex control, where there is not independent control of different tissue interface components. For example, in optogenetic applications, wireless control is limited to illumination at a single targeted region at a single optical wavelength. Accordingly, there is a need for fully implantable wireless devices that have multiplex capability, wherein there is the ability to simultaneously control any number of independently addressable tissue interface components. In this manner, for optical applications, complex optical emissions are achievable in a time-varying, spatial varying, and wavelength dependent manner.

### SUMMARY OF THE INVENTION

Provided herein are fully implantable soft medical devices that do not suffer the disadvantages of conventional implants that require physical interaction or connection with the external environment. For example, conventional optogenetic devices often require remote light sources and attendant fiber optic delivery to provide the desired optical excitation to tissue. Even alleged “wireless” systems require rigid externally positioned fixtures that mount to stable skeletal features, such as the skull. Such devices are vulnerable to physical damage, discomfort to the patient and practical constraints as to device operational lifetime and accessibility to tissue.

Accordingly, provided herein are fully implantable soft medical devices that are capable of full functionality without physical connections to externally located components. The device may comprise: an elastomeric substrate and a stretchable electronic device supported by the substrate. The electronic device may also be considered flexible, in that the electronic device is capable of accommodating substantial levels of strain and bending without failure, thereby providing conformability without adversely impacting functionality. The electronic device may comprise electronic components configured to interface with biological tissue and a wireless power and control system for wirelessly powering and receiving a control signal for controlling the electronic components. An elastomeric superstrate may cover at least a portion of a top surface of the stretchable electronic device. In this manner, the device as a whole is considered “soft” in that the bulk properties such as Young's modulus and bending modulus are tailored to minimize the transmission of undue physical stresses and forces on surrounding tissue. Similarly, the elastomeric covering layers minimizes the effects of any sharp edges, drop-offs, or other relief feature geometry associated with the electronic devices of the medical device. This can be particularly relevant depending on the position of the implant, such as a portion of electronic device implanted between a rigid tissue, such as the skull, and a mechanically vulnerable soft tissue, such as skin.

The wireless power and control system may be provided as a unitary component, such as an NFC chip device. The wireless power and control system may also be described in terms of individual components, such as a radio frequency antenna, including a stretchable radio frequency antenna, in electrical connection with various supporting elements such as impedance matching circuits and voltage multiplier or amplifier to ensure a desired signal is provided to the electronic component that interfaces with the tissue. A common function of any of the power and control systems useful in the instant invention is that it provides wireless control and measurement and does not adversely impact the soft and conformable nature of the implanted device. Exemplary configurations that achieve this functionality include a geometry that is thin with specially shaped serpentine configurations of certain circuit portions.

For example, the wireless power and control system may comprise a stretchable radio frequency antenna having a plurality of adjacent serpentine electrical conductors separated by a separation distance, wherein adjacent serpentine electrical conductors are capacitatively coupled to each other. The plurality of adjacent serpentine electrical conductors may be configured to provide a bandwidth of between 200 MHz and 300 MHz with a center frequency of between 2 GHz and 2.5 GHz over a strain range of up to 25% in a horizontal, a vertical or a horizontal and vertical direction.

The electronic device may be described in terms of a thickness, such as a thickness less than or equal to 100 μm, and the medical device as a whole as having a total thickness, such as a thickness less than 5 mm, lest than 2 mm, or less than 1 mm.

The power system is configured to facilitate wireless control and powering of the device. For example, a magnetic loop antenna and an externally located electrode for generating an electric field over the magnetic loop antenna may be used to power the electronic device.

Any of the systems provided herein may be battery free or be battery-assisted. For example, a combination of wireless powering to recharge batteries and/or power the device may be used.

The control system may comprise an externally located transmitter configured to transmit the control signal to the electronic device and a radio frequency harvester operably connected to the electronic device for receiving the control signal and subsequent control of said electronic components. The control signal may be a radiofrequency wave, with certain frequency corresponding to frequencies for device control. Accordingly, the control system may further comprise an impedance matching circuit and a voltage multiplier, wherein a received power from the power system is converted into a direct current output by the impedance matching circuit and voltage multiplier for the control of electronic components.

The radio frequency harvester may comprise a stretchable radio frequency antenna, such as a stretchable radio frequency antenna having adjacent serpentine electrical conductors in capacitative connection with each other.

The devices provided herein are useful for a range of applications, with various sensors and actuators selected as active electronic device components depending on the application of interest. Exemplary applications include, but are not limited to, any one or more of: electrical stimulation, electrical monitoring, or both; optical stimulation, optical monitoring, or both; controlled delivery of a biotherapeutic agent; thermal control or sensing; or pressure sensing.

Any of the devices provided herein may be configured to interface with a nerve or neural tissue. For example, the device may be configured to interface with a peripheral nerve.

The electronic components may comprise a light source to provide a rapid and temporally controllable optical stimulation. Particularly in multi-channel applications, any number of spatial optical stimulation patterns, with spatially-varying optical wavelengths, may be generated.

The device may be described in terms of an average optical output power density, including a density of between 9.5 mW/mm² and 10 mW/mm² over a target region during device activation and configured for use in an optogenetic application.

Any of the active electronic devices may be micro-sized, such as light sources that comprise one or more μLEDs (micro-LEDs), wherein at least one dimension of the micro-sized device is less than 1 mm, less than 0.5 mm, or less than 0.1 mm, including on the cellular scale, for individual addressing on the cellular scale. Of course, the devices and methods provided herein are scalable to any of a variety of size ranges, depending on the application of interest.

The devices provided herein may be configured for slideable insertion into a muscle pocket of a living animal. Similarly, the leading edge of the device may have a needle shape, thereby providing the capability of implantation in a manner corresponding to injection.

The device may further comprise a pair of bilateral wings connected to or extending from the device and configured for suturing to a surrounding tissue for stable positioning of the device after implantation. For example, the wings may extend from an electrical connector portion that electronically connects the active electronic component(s) that interface with tissue to the back-end power and control system.

The devices provided herein may be configured for chronic wireless implantation and remote control, including for up to one year.

The devices may be described as having a bulk Young's modulus that is matched to soft tissue and is less than or equal to 5 MPa and a bending stiffness of per unit width that is less than or equal to 10⁻⁷N m.

The device may be described as having a device footprint area, such as corresponding to the substrate surface area, including an area that is less than or equal to 500 mm² and, optionally, greater than or equal to 1 mm².

The substrate and/or superstrate may have an average Young's modulus that is independently less than or equal to 10 MPa and, optionally, greater than 0.5 kPa. Other exemplary ranges include between 10 kPa and 100 kPa.

The electronic components may comprise a plurality of independently addressable electronic components for a plurality of independently addressable interfacing with biological tissue. For example, an array of LEDs may be independently addressed to provide desired wavelengths and/or optical output patterns.

The electronic components may comprise at least one actuator and at least one sensor to provide simultaneous and independent control of tissue activation with the actuator and tissue sensing with the sensor.

The device may include electronic components that comprise a plurality of independently addressable LED optical sources.

The addressable LED optical sources can be each independently characterized by an emitting area less than or equal to 1×10⁵ μm², as may other actuator-type electronic components for interfacing with tissue. The independently addressable electronic components, including LED optical sources, may be each independently characterized by an emitting area selected from the range of 1×10³ μm² to 1×10⁵ μm².

The independently addressable electronic components, such as LED optical sources, may be provided in a 1D or 2D array.

The independently addressable electronic components, such as independently addressable LED optical sources, may be operationally connected to a plurality of stretchable antenna structures providing for independent control of the independently addressable electronic components, such as LED optical sources.

The stretchable antenna structures may integrate multiple capacitive coupling traces to provide non-overlapping resonance frequencies for selective energy harvesting and control of an input radiofrequency.

At least a portion of the independently addressable LED optical sources provide light characterized by a different emission wavelength spectrum. In this manner, different color LEDs may be independently controlled.

The plurality of independently addressable electronic components may comprise up to eight independently addressable electronic components for an up to eight-channel multiplexing. Of course, as desired, any number of channels with corresponding multiplexing may be utilized. This provides good flexibility, depending on the application of interest, with independent control of, for example, illumination, electrical activation, thermal and corresponding sensing thereof.

Any of the devices may further comprising a motion tracking system in operational connection with said stretchable electronic circuit for tracking a motion of the devices when in use. The motion tracking system may provide for a confined power delivery to the device over a power area.

The device may further comprise a biodegradable needle operationally connected to the electronic circuit, the biodegradable needle having a stiffness sufficient to allow for injection or implantation of the device in a biological tissue.

Any of the devices described herein may have a wireless power and control system that is stretchable and capable of accommodating a strain that is greater than 10% without fracture, including maximum strains of between 10% and 30%, or between 10% and 20%.

Also provided are methods of interfacing with biological tissue using any of the devices described herein. For example, the interfacing may be by providing in a patient a soft medical device comprising: an elastomeric substrate; a stretchable electronic device supported by said substrate, wherein said stretchable electronic device comprises: one or more electronic components configured to interface with biological tissue; a wireless power and control system for wirelessly powering and receiving a control signal for controlling said electronic components; an elastomeric superstrate that covers at least a portion of a top surface of said stretchable electronic device; and generating a control signal with an externally located control signal generator to wirelessly control said electronic device, thereby interfacing with biological tissue adjacent to the soft medical device. The “externally located” refers to a position of a component that is outside the body where the implant is implanted and not in direct physical contact with the component, including at a distance from the body.

The method may further comprise the step of powering the medical device by an externally-generated radiofrequency signal. The externally generated control signal may be received and processed by a medical device with an NFC device that is operably connected to the electronic device.

The soft medical device may interface with a peripheral nerve tissue, be epidurally implanted for a pain relief application, or may have interfacing with biological tissue by one or more of: optically interfacing; electrically interfacing, thermally interfacing; chemically interfacing; or pressure interfacing.

The interfacing may comprise optical stimulation of a tissue adjacent to the soft medical device, wherein at least a portion of the cells in the tissue have been genetically transformed to express light-sensitive proteins having a light-intensity dependent functional activity.

The soft medical device may be configured to conform to a desired tissue or to a desired shape upon implantation without substantial impact on device functionality. In this context, “substantial” refers to device functionality that deviates by less than 20% from corresponding functionality without conformation change.

The device may also be described in terms of accommodating flexibility in terms of accommodating a radius of curvature that is as small as 1 mm, 0.5 mm, or 50 μm without substantial degradation, including as define above.

The electronic components may comprise a plurality of independently addressable electronic components, the method further comprising the step of independently wirelessly powering and controlling the plurality of independently addressable electronic components. In this manner, multiplexing may be reliably achieved.

The electronic components may comprise a plurality of independently addressable LED optical sources, the method further comprising the step of independently wirelessly powering at least one of the plurality of independently addressable LED optical sources to provide optical stimulation.

The method may further relate to simultaneous device control in multiple patients, each having an implanted soft medical device, the method further comprising the step of: providing a multiplex control for the plurality of medical devices in the plurality of patients.

Also provided herein is a multi-channel fully implantable medical device comprising: a substrate; an electronic device supported by said substrate, wherein said electronic device comprises: a plurality of independently addressable electronic components configured to interface with biological tissue; a multi-channel antenna in electronic contact with said plurality of independently addressable electronic components for controlling said electronic components; and a superstrate that at least partially covers said electronic device. In this manner, the device is considered configured for multiplexing, that is independent and simultaneous control, including based on different electromagnetic frequencies input to the device, including by a wireless connection.

The electronic components may comprise a plurality of actuators, a plurality of sensors, or at least one actuator and at least one sensor, wherein each of the electronic components are independently addressable. The electronic components may comprise a plurality of independently addressable LED optical sources.

At least one LED optical source may have an emission output wavelength maximum that is at least 40 nm different from another LED optical source emission output wavelength maximum, thereby providing multiplex control of different color LED optical sources, for example, red, green and/or blue colored LEDs.

The multi-channel antenna may comprises: a plurality of capacitative coupling traces operably connected to said plurality of electronic components to provide non-overlapping resonance frequencies for selective energy harvesting and independent control of each of the plurality of independently addressable electronic components.

The device stretchability may correspond to a functional definition, such as capable of accommodating a strain greater than 10% without device failure and/or bending down to 5 mm, 2 mm, 1 mm, 0.1 mm or 50 μm radius of curvature.

The substrate and superstrate may comprise an elastomeric material having a Young's modulus of less than 10 MPa.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A-1I. Ultraminiaturized, fully implantable, soft optoelectronics systems for wireless optogenetics. FIG. 1A Exploded view schematic illustration of the energy harvester component of the system, with an integrated LED to illustrate operation. FIGS. 1B and 1C Illustration of the anatomy and location of the device relative to the sciatic nerve and spinal cord, respectively. FIG. 1D Picture of a harvester laminated onto the tip of the index finger. The device is 0.7 mm thick, 3.8 mm wide, and 6 mm long; its weight is 16 mg. FIG. 1E Picture of the epidural device embodiment, highlighting the soft, stretchable connection to an LED. The diameter of the injectable component is 380 □m, with cross sectional dimensions comparable to the epidural space. FIGS. 1F and 1G Images of mice with wireless devices implanted near the sciatic nerve and the spinal cord, respectively. FIGS. 1H and 1I Strain distributions in the stretchable antenna (left) and its reflection coefficient (right) for the cases of strain applied in the horizontal (28%) and vertical directions (30%) (blue), respectively, and for the undeformed configuration (red dashed).

FIG. 2A-2H. Radio frequency characteristics and control strategies. FIG. 2A Schematic illustration of the TX system and an experimental assay with computed SAR distributions on a mouse mesh body. Multiple antennas lie in the XY plane, placed below the assay. FIG. 2B Transmission coefficients (S₁₂) as a function of frequency for orientation in the XY, YZ, and ZX planes. FIGS. 2C, 2D and 2E Angular radiation beam patterns of the TX system (blue) and implanted antennas (red) located in the XY, YZ, and ZX planes. FIG. 2F Available power for operating the devices. The dashed line in the scale bar identifies the minimum power for operation. FIG. 2G Simultaneous operation of devices implanted into multiple animals in the same cage (30 cm by 30 cm). FIG. 2H Pictures of continuous streaks of light in the box (blue LEDs—left; green LEDs—right), corresponding to experiments in which mice freely move (with manually induced trajectories) in an enclosure during continuous operation of implanted devices.

FIG. 3A-3G. Electrophysiological and anatomical characterization of ChR2 expression in Advillin-ChR2 mice. FIG. 3A Schematic of the Ai32 locus and Advillin-Cre mouse locus, together with results of the credependent recombination of the Ai32 locus. FIGS. 3B and 3C Electrophysiological recordings from DRG neurons cultured from Advillin-ChR2 mice. For all traces, 470 nm illumination is delivered at 10 mW/mm². FIG. 3B 1 second-long illumination induces inward currents (lower trace) in voltage clamp, and in some cells produces sustained firing in current clamp recordings (upper trace). FIG. 3C Pulsed illumination at 20 Hz induces action potential firing with high fidelity (upper trace) resulting from the inward currents that are consistently generated as demonstrated in voltage clamp (lower trace). Note that the first pulse produces larger amplitude inward currents relative to the 2nd and all subsequent pulses of light, consistent with the rapid desensitization to a steady state current seen with prolonged illumination (FIG. 3B, lower). FIG. 3D Immunohistochemical analysis of tissue from adult Advillin-Ai32 mice demonstrates that ChR2 is expressed along the peripheral neuraxis, including termination in lamina I and lamina Hof the spinal cord dorsal horn as evidenced by overlap with CGRP (purple) and IB4 (red), respectively. FIG. 3E Staining of DRG demonstrates significant overlap of ChR2 expression with βIII tubulin (purple) and IB4 (red) within the soma. Longitudinal (FIG. 3F) and cross sections (FIG. 3G) of sciatic nerve demonstrate robust staining along the plasma membrane of the axons. See FIG. 17A-17F for comparison to the ChR2 expression pattern seen in TRPV1-Ai32 mice which, as expected, is more restricted than in these Advillin-ChR2 mice. Scale bars=100 μm.

FIG. 4A-4J. Wireless activation of ChR2 expressed in nociceptive pathways results in spontaneous pain behaviors and real-time place aversion. FIG. 4A Representation of ascending nociceptive pathways and illumination of nociceptive fibers with a sciatic μLED stimulator. FIG. 4B Implantation of the sciatic μLED stimulator has no effect on motor behavior vs. sham animals in the rotarod test (p=0.894, n=5 sham, n=8 device). FIG. 4C Wireless activation of the sciatic μLED stimulator caused increased nocifensive behaviors (flinching, hind paw licking, jumping) in Advillin-ChR2 mice (p<0.0001, n=3) but not in controls (n=3). FIG. 4D Real Time Place Aversion (RT-PA) Schematic. FIG. 4E Heat maps represent the time spent in each zone. In animals implanted with the sciatic μLED device, aversion to the LED-ON zone is observed in TrpV1-ChR2 and Advillin-ChR2 mice, but not in controls. FIG. 4F Quantification of time spent in each zone during RT-PA. TrpV1-ChR2 (p=0.011, n=5) and Advillin-ChR2 (p=0.032, n=8) mice display aversion to the LED-ON zone vs. the LED-OFF zone. No difference is observed in control mice (p=0.551, n=10). FIG. 4G Representation of ascending nociceptive pathways and illumination of primary afferent terminals innervating the spinal cord with a wireless epidural implant. FIG. 4H Wireless activation of the epidural μLED implant increased nocifensive behaviors in SNS-ChR2 mice (p<0.001, n=3 vs control mice n=3). FIG. 4I Heat maps represent time spent in each zone during RT-PA. Aversion to the LED-ON zone was observed in SNS-ChR2 mice, but not in controls. FIG. 4J Quantification of time spent in each zone during RT-PA. SNSChR2 mice display aversion to the LED-ON zone (p=0.006, n=3). No difference was observed in control mice (n=3).

FIG. 5A-5B. Layouts and components information of (FIG. 5A) Sciatic and (FIG. 5B) spinal epidural devices.

FIG. 6A-6C. Overview of system characteristics. FIG. 6A The peak wavelength of blue LED and the channelrhodopsin, FIG. 6B Current-Voltage (I-V) characteristics of LED, FIG. 6C light output power as a function of electrical input current.

FIG. 7A-7D. Demonstrations of wireless operation when a harvester is completely folded in half (FIG. 7A), rolled up (FIG. 7B), twisted (FIG. 7C), and knotted (FIG. 7D), respectively.

FIG. 8A-8B. Images of a mouse with a blue LED (FIG. 8A) or a green LED (FIG. 8B) during exercise on a running wheel.

FIG. 9A-9B. Hematoxylin and eosin (H&E) staining of sciatic nerves taken from C57BL/6J wildtype mice at 16× magnification. FIG. 9A is a representative ipsilateral nerve which has been interfaced with the sciatic optogenetic simulator. FIG. 9B is the contralateral nerve from this same representative animal utilized for comparison. No gross infiltration was noted using standard H&E techniques of neutrophils, lymphocytes, monocytes, basophils, eosinophils, red blood cells, or lipofuschin.

FIG. 10A Exploded view of a stretchable antenna (top), schematic view of constituent serpentine wires (bottom). FIG. 10B Normalized electric field distributions on a stretchable antenna (top), and exploded view (bottom). Five pairs of adjacent serpentine lines allow estimate of variations in gap distance with applied strain. FIG. 10C Reflection coefficient, S11, of a stretchable antenna in its undeformed configuration.

FIG. 11A-11H. Mechanical characteristics of the stretchable antenna when strain applied in the vertical direction (FIGS. 11A, 11C, 11E and 11G), and in the horizontal direction (FIGS. 11B, 11D, 11F and 11H). FIG. 11A Six, FIG. 11B five pairs of serpentine lines were selected, respectively. FIGS. 11C and 11D Plots of gap variations versus strain on the antenna. FIGS. 11E and 11F Images of the stretched devices. FIGS. 11G and 11H Plots of center frequency versus strain on antenna of experiments and simulations.

FIG. 12A-12B. Characteristics of the stretchable antenna under wet and dry skin. FIG. 12A Reflection coefficient, S11, of the stretchable antenna (under wet skin in red and dry skin in blue) FIG. 12B Deviations of center frequencies as a function of strain (under wet skin in red and dry skin in blue).

FIG. 13. Thermal characteristics of the fully implantable harvester. Variations of temperature were monitored in various optical output densities.

FIG. 14A-14C. Illustrations of mouse motions when the mouse is located on the (FIG. 14A) XY, (FIG. 14B) YZ, and (FIG. 14C) ZX plane, respectively.

FIG. 15A-15E. Overview of RF control strategies for spinal cord stimulation. FIG. 15A Schematic view of the TX system and an experimental assay with SAR distributions on a mouse mesh body. Multiple antennas are located on the XY plane and placed below the assay. FIGS. 15B, 15C and 15D Transmitted beam patterns of the TX system and the harvester implanted when the harvester (or a mouse) is located in the XY, YZ, and ZX plane. FIG. 15E Transmission characteristics, transmission coefficients (S₁₂) of the TX system and a freely moving mouse in an assay when the mouse is located in XY, YZ, and ZX plane, respectively.

FIG. 16. Map of optical power densities in an experimental assay (30 cm by 30 cm).

FIG. 17A-17F. Electrophysiological and anatomical characterization of ChR2 expression in TRPV1-Ai32 mice. FIG. 17A Schematic of the Ai32 locus and TRPV1-Cre mouse locus, together with results of the credependent recombination of the Ai32 locus. FIG. 17B Inward current in cultured sensory neuron from the TRPV1-ChR2 mice. FIG. 17C In current clamp, 20 Hz pulsed illumination results in high-fidelity action potential firing in TRPV1-ChR2 mice. FIG. 17D Immunohistochemical analysis of tissue from adult TRPV1-Ai32 mice demonstrates that ChR2 is expressed along the peripheral neuraxis, including termination in lamina I and lamina II of the spinal cord dorsal horn as evidenced by overlap with CGRP (purple) and IB4 (red), respectively. FIG. 17E Staining of DRG demonstrates significant overlap of expression with CGRP (purple) and IB4 (red) within the soma, and longitudinal (FIG. 17F) and cross sections (FIG. 17G) of sciatic nerve demonstrate robust staining along the plasma membrane of the axons. This expression pattern, as expected is more restricted than in Advillin-Ai32 mice. Scale bars=100 μm.

FIG. 18A. Accelerating rotarod testing of C57BL/6J wildtype mice implanted with epidural device (device) versus sham-operated littermates (sham) at one week post-implantation (n=6). Two-way ANOVA revealed no significant difference (p=0.226) between the two groups. FIG. 18B. Open field testing of locomotor activity of C57BL/6J wildtype mice implanted with optogenetic sciatic nerve stimulator versus sham-operated littermates at one week post-implantation (n=13). T-test revealed no significant difference between the two groups (p=0.886). FIG. 18C. Open field testing of locomotor activity of C57BL/6J wildtype mice implanted with epidural device implanted versus sham-operated littermates at one week post-implantation (n=6). T-test revealed no significant difference between the two groups (p=0.590).

FIG. 19. Glial fibrillary acid protein (GFAP) representative immunohistochemistry from 40 um spinal cord sections taken from C57BL/6J wild type mice implanted with the epidural implant (lower), versus sham-operated control littermates (upper) after one week of recovery.

FIG. 20A-20D. Multi-channel, soft wireless optoelectronic systems for optogenetics. FIG. 20A Exploded view schematic illustration of the multi-channel energy harvester components of the system. FIG. 20B Schematic illustration of the anatomy and location of the device relative to the brain. FIG. 20C Pictures of a two-channel, operating device on a fingertip, deformed by application of localized force with a pair of tweezers to illustrate the soft mechanics (left) and wireless operation in the air (right top and bottom). This harvester activates a green μ-ILED at 2.3 GHz (Channel 1, right to the top) and a blue μ-ILED at 2.7 GHz (Channel 2, right to the bottom). FIG. 20D Images of mice implanted with the two-channel optoelectronic system, operating at a frequency of 2.3 (top) and 2.7 (bottom) GHz, respectively.

FIG. 21A-21F. Overview of electrical and optical characteristics of a two-channel stretchable antenna. FIG. 21A Schematic illustration of a two-channel stretchable antenna (left) and red dotted magnified view of two input ports (right). Here serpentine lines are highlighted by colors (blue, red, and black), which makes it easy to identify which line contributes to a channel. Operation at Channel 1 is associated with capacitive coupling between blue and red lines; operation at Channel 2 is related to the coupling between red and black lines. FIG. 21B Normalized magnitude of electric fields on the serpentine lines (left) at a frequency of 2.3 GHz. Magnified views, corresponding to the regions highlighted by the black and red dotted boxes, show capacitive coupling between adjacent serpentine lines (blue, red, and black). FIG. 21C Scattering parameters of the two-channel stretchable antenna. FIG. 21D Block diagram of the two-channel stretchable optoelectronic system. Channel 1 and 2 operate at frequencies of 2.3 and 2.7 GHz, respectively. FIGS. 21E and 21F Measurements of the optical intensity generated in the non-targeted channel as a function of the optical intensity in the targeted channel at a frequency of 2.3 and 2.7 GHz, respectively. The dotted line identifies the threshold radio frequency power required to activate the non-targeted μ-ILED in each channel.

FIG. 22A-22K. Electrical characteristics of a three-channel stretchable antenna. FIG. 22A Schematic of a three-channel stretchable antenna with a magnified view of the antenna. The antenna consists of 4 serpentine lines (red, black, blue, and green). Here serpentine lines are highlighted by colors (red, black, blue, and green), which makes it easy to identify which line contributes to a channel. Channel 1, 2, and 3 are tuned at a frequency of 2.3, 2.7, and 3.2 GHz, respectively. Operation at Channel 1 is associated with capacitive coupling between red and black lines, operation at Channel 2 is related to the coupling between black and blue lines, and operation at Channel 3 is connected with the coupling between blue and green lines. FIG. 22B Normalized magnitude of electric fields on the serpentine lines at a frequency of 2.3 GHz. Enlarged views of it show capacitive coupling between adjacent serpentine lines. FIG. 22C Scattering parameters of the three-channel antenna. Each dotted line indicates an operation frequency at each channel. FIG. 22D-22F Measurements of optical intensity generated in the non-targeted channels as a function of the optical intensity in the targeted channel at a frequency of 2.3 (FIG. 22D), 2.7 (FIG. 22E), and 3.2 (FIG. 22F) GHz respectively. Dotted lines represent threshold power required for activation of the non-targeted μ-ILEDs in each channel. This can be referred to as a maximum single-channel activation threshold. FIG. 22G Images of wireless operation of the three-channel stretchable antenna at a frequency of 2.3 (top; green), 2.7 (middle; blue), and 3.2 GHz (bottom; red), respectively. FIG. 22H Angular radiation patterns of the three-channel stretchable antenna at a frequency of 2.3 (left), 2.7 (middle), and 3.2 GHz (right), respectively. FIG. 22I Cross sectional view of (h) at θ=90°. FIG. 22J Variations of center frequencies in terms of strain applied at each channel. FIG. 22K Comparison of area of one antenna with multi-channel to that of multi-antennas systems.

FIG. 23A-23H. Analytical modeling of the capacitive coupling and investigations of the input impedance as a function of input port location. FIG. 23A Schematic illustration of serpentine lines approximated with sinusoidal curves. The top (bottom) line is biased at V₀ (−V₀). The red dotted line represents a unit cell of a pair of serpentine lines. FIG. 23B Equivalent circuit model where the serpentine lines are treated as a sum of infinitely small capacitors where with top and bottom plate width of dx, separated by a gap of y. εᵣ and ε₀ are relative permittivity and permittivity of the air, respectively. FIG. 23C Magnitude of electric fields (numerical simulations in black; analytical modeling in red) along the line (Y=h−I). Here, h=500 μm & I=380 μm. FIG. 23D Unit capacitance (numerical simulations in black; analytical modeling in red) as a function of gap between adjacent serpentine lines. FIG. 23E Schematic illustration of a stretchable antenna. Each number represents the location of an input port. FIG. 23F Impedance as a function of input port location. FIG. 23G Scattering coefficients, S₁₁, of a stretchable antenna at each port. FIG. 23H Power reflection as a function of input port. Numbers represent how much electromagnetic waves are reflected at each input port, where low values are highly desirable.

FIG. 24A-24F. Single-channel optogenetic studies of complex behavioral responses. FIG. 24A Illustration of light illumination to locus coeruleus (LC) in the mouse brain. FIG. 24B Experimental paradigm for testing optogenetic control of arousal. FIG. 24C Confocal micrograph of virally-induced ChR2 expressions in galanin-expressing neurons in the LC region. Tyrosine hydroxylase (TH) immunoreactivity is red and ChR2-eYFP native fluorescence is yellow. FIG. 24D Photograph of hardware setup, six panel antennas line the bottom and side of the homecage. FIG. 24E Representative heatmap of position in homecage during each epoch of the experiment for a Gal-Cre+ animal. Map is only plotted where the animal explored in the homecage (i.e. the animal was stationary in the baseline and post-stimulation epochs), but freely explored during stimulation. FIG. 24F Analysis of the total distance traveled during each epoch in a home cage (n=3-5/group) (****p<0.0001 2-way Repeated measures ANOVA with Bonferroni post-hoc test).

FIG. 25A-25F. Multi-channel optogenetic studies of complex behavioral responses. FIG. 25A Cartoon of directionally-controlled light spread of μ-ILED devices for isolating subregions of NAcSh. FIG. 25B Experimental paradigm for testing optogenetic control of reward and aversion. FIG. 25C Confocal micrograph of virally-induced ChR2 expression in dynorphin-expressing neurons in the NAcSh. Nissl cell body stain is blue and ChR2-eYFP native fluorescence is green. FIG. 25D Photo of the real-time place testing assay. FIG. 25E Representative heatmap of time spent in the stimulation (RF on) side following no stimulation, dorsal or ventral wireless photostimulation and dorsal and ventral photostimulation together. FIG. 25F Stimulation with dorsal μ-ILED drives a real-time place preference but stimulation with ventral μ-ILED drives an aversion, measured as a significant increase or decrease in time spent in the stimulation side (%) respectively. Stimulating both ventral and dorsal μ-ILEDs has no significant effect on behavior. (Data represented as mean±SEM, n=6: One-Way ANOVA, Bonferroni post-hoc; ****p<0.0001 RF off vs ventral and dorsal vs ventral; **p<0.001 dorsal vs both and ventral vs both).

FIG. 26. Layout and component information for the multi-channel devices.

FIG. 27. Assessment of variations in normalized light intensity produced by the multi-channel system as a function of time of immersion in physiological PBS (7.4 pH) solution at three different temperatures.

FIG. 28A Images of a biodegradable, injectable p-needle in physiological PBS solution after 0 day (left), 1 day (middle), and 3 days (right) respectively. Results of measurements of the p-needle thickness (FIG. 28B) and bending stiffness (FIG. 28C) as a function of time of immersion in PBS at a variety of temperatures.

FIG. 29. In vivo monitoring of temperature of a mouse during device operation, for various duty cycles (DC).

FIG. 30. Scattering coefficients, S₁₁, of a 4-channel stretchable antenna. The antenna has 4 operation channels at a frequency of 1.9 (black), 2.2 (red), 2.8 (blue), and 3.2 (green) GHz, respectively.

FIG. 31A Illustration of motion tracking power transmission systems. A camera monitors the location of a mouse and sends images to the base station, which processes the images and sends control signals to the multiplexer (Mux). Output signals from Mux manipulate a switch that activates the appropriate circuit to transmit power with the appropriate antenna. These processes occur within 1 ms which is fast enough to respond to the motions of a mouse instantaneously. FIG. 31B Flowchart of the motion detection algorithm. FIG. 31C Illustration of smart power transmission. The antenna that is closest to the mouse is enabled and transmits power.

FIG. 32. Plot of average TX power as a function of number of antennas. The smart power system distributes a given TX power of 2 W across the antennas.

FIG. 33. Analysis of the total distance traveled, as an assessment of activity level, during a 20 min experiment.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Fully implantable” refers to a device capable of implantation into biological tissue without any portion that extends out of the tissue into the surrounding environment, having full functionality throughout the desired device lifetime. In contrast, non-fully implantable devices contain components that are physically accessible, including for desired device operation, including device control, device data transmission, device powering, or the like.

“Soft” refers to the ability of implanted devices to accommodate natural motion of biological tissue or forces exerted on the implanted device by deformation, thereby minimizing unwanted stresses and resultant forces on surrounding tissue. Soft may be quantifiably defined in terms of a Young's modulus, such as a low Young's modulus, or a Young's modulus less than or equal to 10 MPa. Similarly, the Young's modulus may be substantially matched to that of the soft tissue in which the device is implanted, including within about 10% of a bulk Young's modulus of adjacent tissue.

“Elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers. Elastomeric substrate or superstrate refers to a layer comprising at least one elastomer. Elastomeric substrates may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. In some embodiments, an elastomeric stamp comprises an elastomer. Exemplary elastomers include, but are not limited to silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a polymer is an elastomer.

“Conformable” refers to a device, material or substrate which has a bending stiffness that is sufficiently low to allow the device, material or substrate to adopt any desired contour profile, for example a contour profile allowing for conformal contact with a surface having a pattern of relief features. Similarly, the Young's modulus is sufficiently low or soft so that the device contours to the surrounding tissue, including under an applied force. In certain embodiments, a desired contour profile is that of a tissue in a biological environment.

“Young's modulus” is a mechanical property of a material, device or layer which refers to the ratio of stress to strain for a given substance. Young's modulus may be provided by the expression:

$$E = \frac{( stress )}{( strain )} = ( \frac{{L}_{0}}{Δ L} ) ( \frac{F}{A} ) , \qquad ( I )$$

where E is Young's modulus, L₀ is the equilibrium length, AL is the length change under the applied stress, F is the force applied, and A is the area over which the force is applied. Young's modulus may also be expressed in terms of Lame constants via the equation:

$$E = \frac{μ ( 3 λ + 2 μ )}{λ + μ} , \qquad ( II )$$

where λ and μ are Lame constants. High Young's modulus (or “high modulus”) and low Young's modulus (or “low modulus”) are relative descriptors of the magnitude of Young's modulus in a given material, layer or device. In some embodiments, a high Young's modulus is larger than a low Young's modulus, preferably about 10 times larger for some applications, more preferably about 100 times larger for other applications, and even more preferably about 1000 times larger for yet other applications. In an embodiment, a low modulus layer has a Young's modulus less than 100 MPa, optionally less than 10 MPa, and optionally a Young's modulus selected from the range of 0.1 MPa to 50 MPa. In an embodiment, a high modulus layer has a Young's modulus greater than 100 MPa, optionally greater than 10 GPa, and optionally a Young's modulus selected from the range of 1 GPa to 100 GPa. In an embodiment, a device of the invention has one or more components, such as substrate, encapsulating layer, inorganic semiconductor structures, dielectric structures and/or metallic conductor structures, having a low Young's modulus. In an embodiment, a device of the invention has an overall low Young's modulus.

“Inhomogeneous Young's modulus” refers to a material having a Young's modulus that spatially varies (e.g., changes with surface location). A material having an inhomogeneous Young's modulus may optionally be described in terms of a “bulk” or “average” Young's modulus for the entire material.

“Low modulus” refers to materials having a Young's modulus less than or equal to 10 MPa, less than or equal to 5 MPa or less than or equal to 1 MPa.

“Bending stiffness” is a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

“Stretchable” refers to an electronic device capable of accommodating substantial strain without failure.

To provide good flexibility or stretchability, at least one of the inorganic semiconductor components or one or more metallic conductor components of the electronic device is optionally a flexible or a stretchable structure. The flexible or stretchable structure may be an interconnect that connects island structures, such as island structures or other components that tend to be relatively less stretchable or flexible. In this manner, the interconnects may accommodate stresses and strains associated with stretching or flexing while avoiding undue stress and strain on the one more relatively rigid components, islands, or chips. For example, the stretchable structure may correspond to a relatively thin electrical conductor having a serpentine or meandering geometry.

“Electronic device” generally refers to a device incorporating a plurality of components, and includes large area electronics, printed wire boards, integrated circuits, component arrays, biological and/or chemical sensors, physical sensors (e.g., temperature, strain, etc.), nanoelectromechanical systems, microelectromechanical systems, photovoltaic devices, communication systems, control systems, power systems, medical devices, optical devices and electro-optic devices. An electronic device may sense a property of the target tissue, may control a property of the target tissue, and/or may provide to surrounding tissue a desired physical signal, such as optical stimulation, thermal stimulation, chemical stimulation, and the like. Furthermore, as used herein an “active electronic components” refers to those components of the electronic device that directly provide the desired functional interfacing with the tissue. The active electronic component may be either an actuator or a sensor.

“Actuating element” and “actuator” are used synonymously and refers to a device component useful for interacting with, stimulating, controlling, or otherwise affecting an external structure, material or fluid, for example a biological tissue. Useful actuating elements include, but are not limited to, electrode elements, electromagnetic radiation emitting elements, light emitting diodes, lasers and heating elements. Actuating elements include electrodes for providing a voltage or current to a tissue. Actuating elements include sources of electromagnetic radiation for providing electromagnetic radiation to a tissue. Actuating elements include ablation sources for ablating tissue. Actuating elements include thermal sources for heating tissue. Actuating elements include displacement sources for displacing or otherwise moving a tissue. Actuating elements may also include biotherapeutic devices for controlled delivery of a bioactive agent.

“Sensing element” and “sensor” are used synonymously and refers to a device component useful as a sensor and/or useful for detecting the presence, absence, amount, magnitude or intensity of a physical property, object, radiation and/or chemical. Sensors in some embodiments function to transduce a biological signal into an electrical signal, optical signal, wireless signal, acoustic signal, etc. Useful sensing elements include, but are not limited to electrode elements, chemical or biological sensor elements, pH sensors, optical sensors, photodiodes, temperature sensors, capacitive sensors strain sensors, acceleration sensors, movement sensors, displacement sensors, pressure sensors, acoustic sensors or combinations of these.

“Interface” is used broadly herein to refer to the interplay between electronic components of the electronic device and biological tissue in which the device is implanted. For example, interface includes an electronic component that generates a physical parameter or signal to affect biological tissue, ranging from optical, thermal, electrical or chemical. Interface may also refer to an electronic component that measures a parameter of the tissue. Interface may also refer to both. For example, an optical light source may provide excitation light to a tissue, and an optical detector to detect light emitted by the tissue, or a flourophor associated with the tissue, at a desired wavelength. The multiplex capability of devices provided herein is a flexible platform for generating any one or more physical parameters, including light, temperature, electrical field, chemical and also optionally assessing tissue status. Provided herein is a flexible mix and match of sensor types, actuator types, and both.

“Power and control” system is used broadly herein to refer to those portions of the electronic device responsible for controlling and powering active components of the electronic device, such as optical light sources, thermal actuators, chemical or biotherapeutic release devices, or any other component requiring an electrical signal and/or power to operate. For example, a near field communication chip device may be used to wirelessly receive command and control signals from a controller physically separated from the implanted device.

“Encapsulate” refers to the orientation of one structure such that it is at least partially, and in some cases completely, surrounded by one or more other structures, such as a substrate, adhesive layer or encapsulating layer. “Partially encapsulated” refers to the orientation of one structure such that it is partially surrounded by one or more other structures, for example, wherein 30%, or optionally 50% or optionally 90%, of the external surfaces of the structure is surrounded by one or more structures. “Completely encapsulated” refers to the orientation of one structure such that it is completely surrounded by one or more other structures. The invention includes devices having partially or completely encapsulated inorganic semiconductor components, metallic conductor components and/or dielectric components, for example, via incorporation a polymer encapsulant, such as biopolymer, silk, a silk composite, or an elastomer encapsulant. The encapsulation may correspond to a substrate that supports an electronic device and a superstrate that covers the electronic device.

“Barrier layer” refers to a component spatially separating two or more other components or spatially separating a component from a structure, material, fluid or environment external to the device. In one embodiment, a barrier layer encapsulates one or more components. In some embodiments, a barrier layer separates one or more components from an aqueous solution, a biological tissue or both. The invention includes devices having one or more barrier layers, for example, one or more barrier layers positioned at the interface of the device with an external environment.

“Thermal contact” refers to the ability of two or more materials and/or structures that are capable of substantial heat transfer from the higher temperature material to the lower temperature material, such as by conduction. Thermal communication refers to a configuration of two or more components such that heat can be directly or indirectly transferred from one component to another. In some embodiments, components in thermal communication are in direct thermal communication wherein heat is directly transferred from one component to another. In some embodiments, components in thermal communication are in indirect thermal communication wherein heat is indirectly transferred from one component to another via one or more intermediate structures separating the components.

“Fluid communication” refers to the configuration of two or more components such that a fluid (e.g., a gas or a liquid) is capable of transport, flowing and/or diffusing from one component to another component. Elements may be in fluid communication via one or more additional elements such as tubes, containment structures, channels, valves, pumps or any combinations of these. In some embodiments, components in fluid communication are in direct fluid communication wherein fluid is capable of transport directly from one component to another. In some embodiments, components in fluid communication are in indirect fluid communication wherein fluid is capable of transport indirectly from one component to another via one or more intermediate structures separating the components.

“Electrical contact” refers to the ability of two or more materials and/or structures that are capable of transferring charge between them, such as in the form of the transfer of electrons or ions. Electrical communication refers to a configuration of two or more components such that an electronic signal or charge carrier can be directly or indirectly transferred from one component to another. As used herein, electrical communication includes one way and two way electrical communication. In some embodiments, components in electrical communication are in direct electrical communication wherein an electronic signal or charge carrier is directly transferred from one component to another. In some embodiments, components in electrical communication are in indirect electrical communication wherein an electronic signal or charge carrier is indirectly transferred from one component to another via one or more intermediate structures, such as circuit elements, separating the components.

“Optical communication refers to a configuration of two or more components such that electromagnetic radiation can be directly or indirectly transferred from one component to another. As used herein, optical communication includes one way and two way optical communication. In some embodiments, components in optical communication are in direct optical communication wherein electromagnetic radiation is directly transferred from one component to another. In some embodiments, components in optical communication are in indirect optical communication wherein an electromagnetic radiation is indirectly transferred from one component to another via one or more intermediate structures, such as reflectors, lenses, or prisms, separating the components.

“Operably connected” refers to a configuration of elements, wherein an action or reaction of one element affects another element, but in a manner that preserves each element's functionality. For example, the action of a radio frequency harvester operably connected to the electronic device ensures that a received signal by the harvester is provided to the active electronic components to a desired tissue interface action, without affecting the functionality of any of the components of the device, or the device operation as a whole.

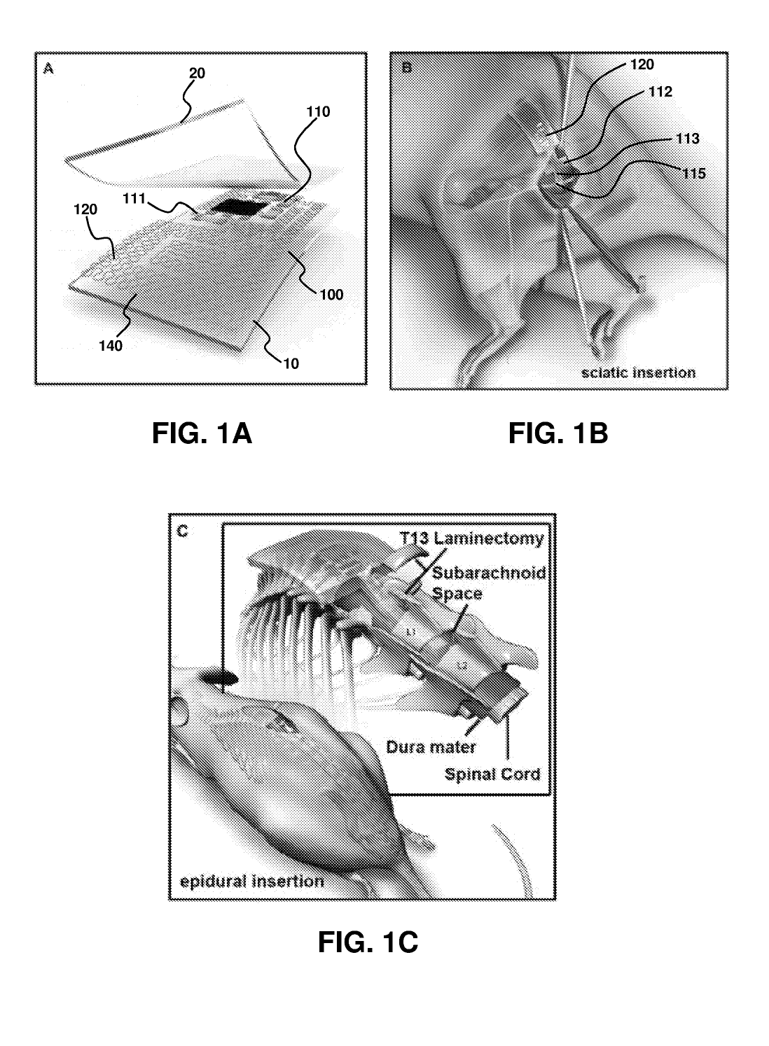

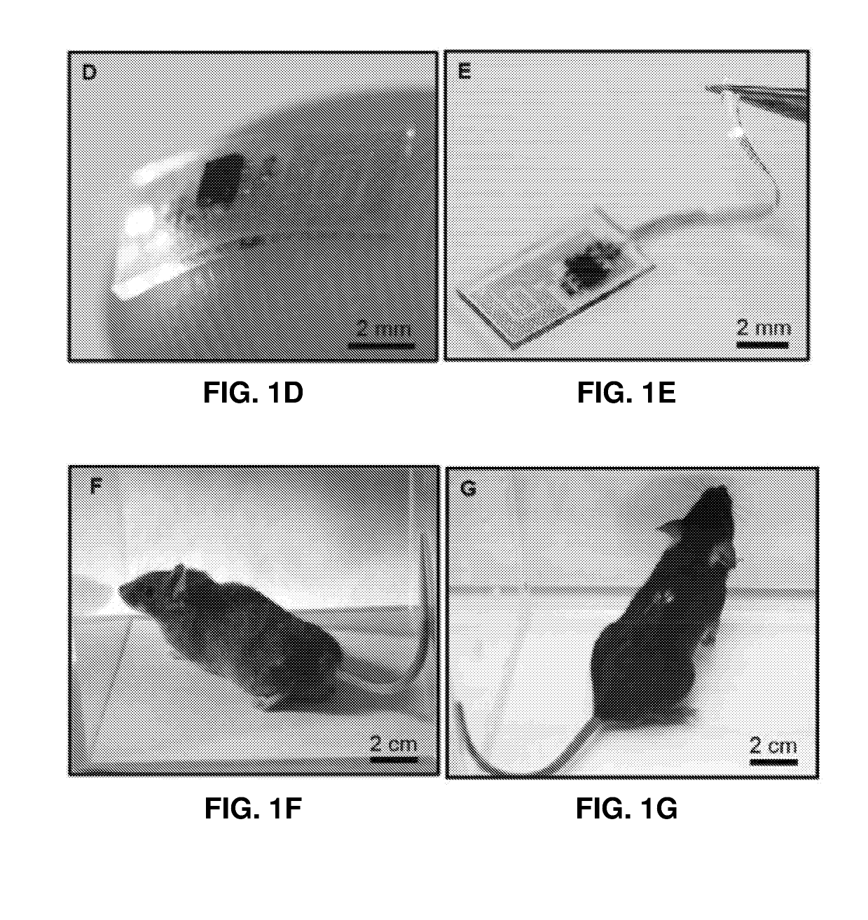

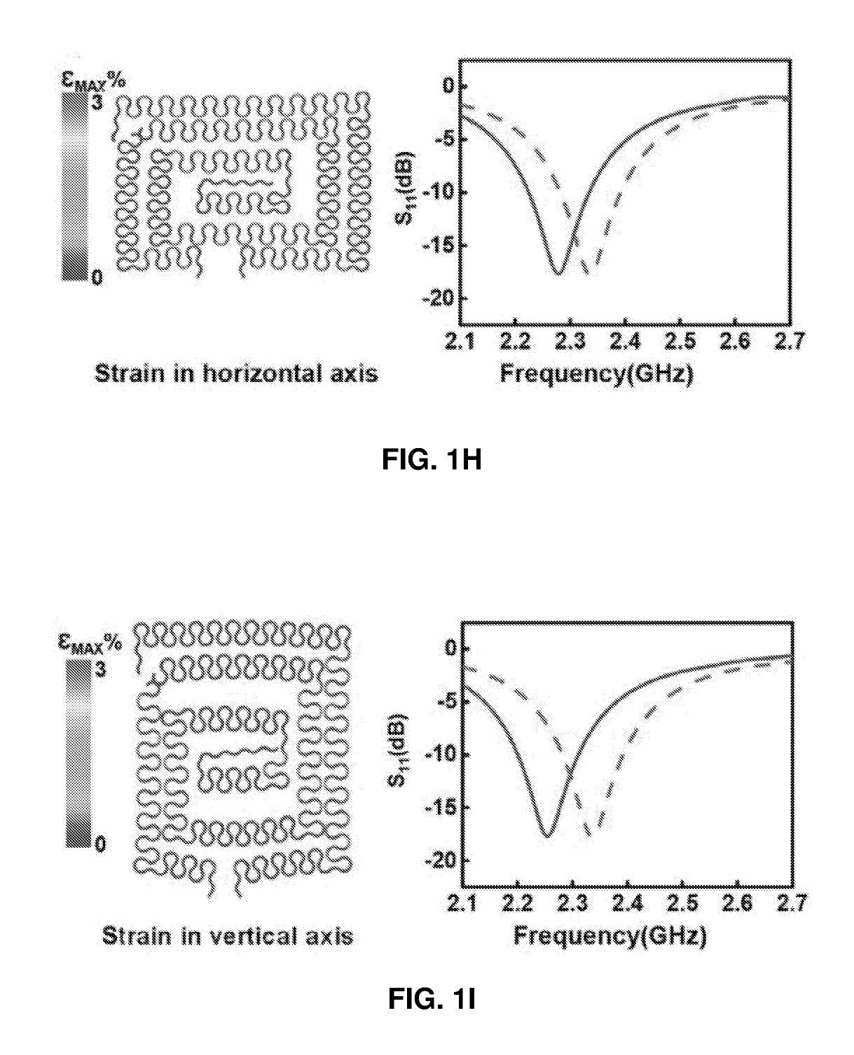

Referring to FIG. 1A, a fully implantable soft medical device has an elastomeric substrate 10 that supports a stretchable electronic device 100. The stretchable electronic device 100 itself may comprise electronic components 110 and a wireless power and control system 120. An elastomeric superstrate 20 may cover at least a portion of a top surface 111 of the stretchable electronic device 110. The stretchable electronic device 100 and substrates 10 and 20 together provide desired stretchability and flexibility, with specially configured thin elements/layers and/or curved structures capable of accommodating strains and corresponding stresses by deformation. In this manner, relatively rigid and inflexible electronic components are protected, and the overall medical device may be characterized as soft and conformable without adversely impacting medical device functionality.

FIG. 1B illustrates that the electronic device may have spatially separated components, with an active electronic component 110 that interfaces with tissue of interest (illustrated as LEDs 115 and nerves, including for optogenetic applications), physically separated from a wireless power and control system 120 and related electronic components 110 operationally positioned therebetween. The wireless power and control system may be positioned in a convenient location with respect to an externally located signal generator that can wirelessly power, control and/or monitor those electronic components. Similarly, the electronic components 115 that interface with the tissue may be independently positioned at a distance from the power and control system 120, specifically adjacent to tissue of interest. A pair of bilateral wings 112 may be used to reliably and stable position the active electronic components that are interfacing with tissue, including via sutures. Connector 113 may electrically and operably connect the active electronic components 115 to the power and control system 120. Such a configuration facilitates desired positioning of different components of the electronic device. As FIG. 1B illustrates use of the device for nervous tissue interfacing, the active components of the system, e.g., LEDs 110 may be positioned adjacent or around the relevant nerve(s), with the back-end power and control system physically separated therefrom, such as positioned immediately below the skin. Such a configuration reflects the different functionalities of the different components of the electronic device. The device is configured to be soft to avoid unwanted tissue damage during use, even under extreme animal movement, including avoiding abrasion of the skin or deeper tissue in which the active portion of the device is implanted.

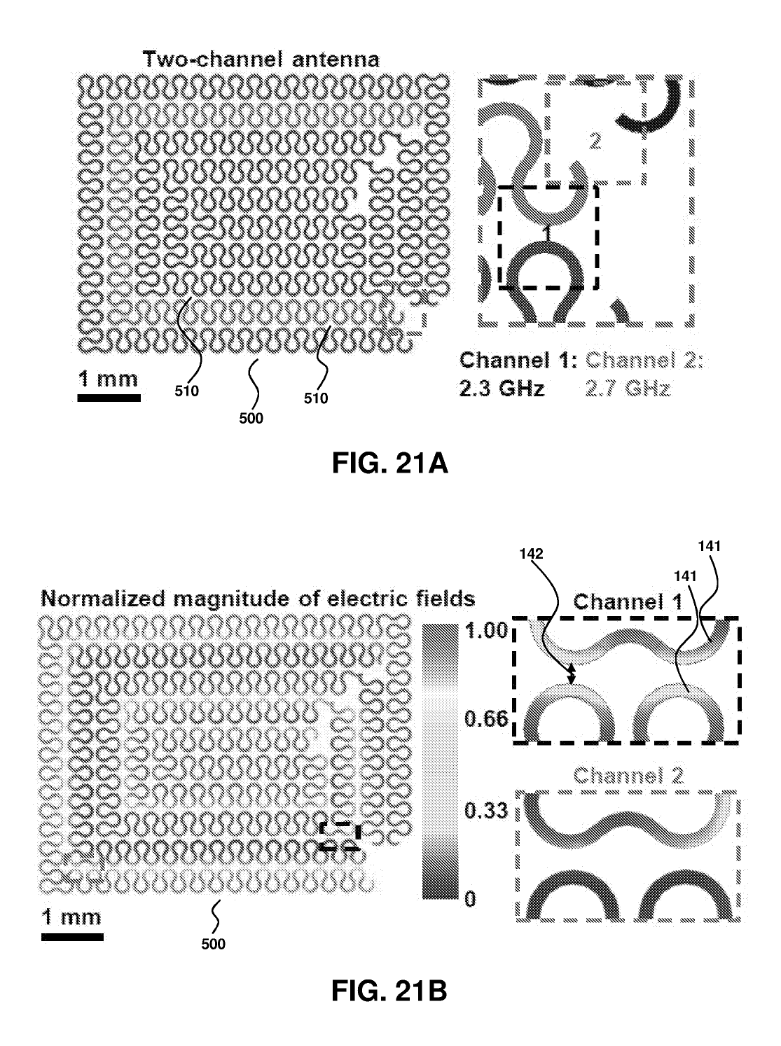

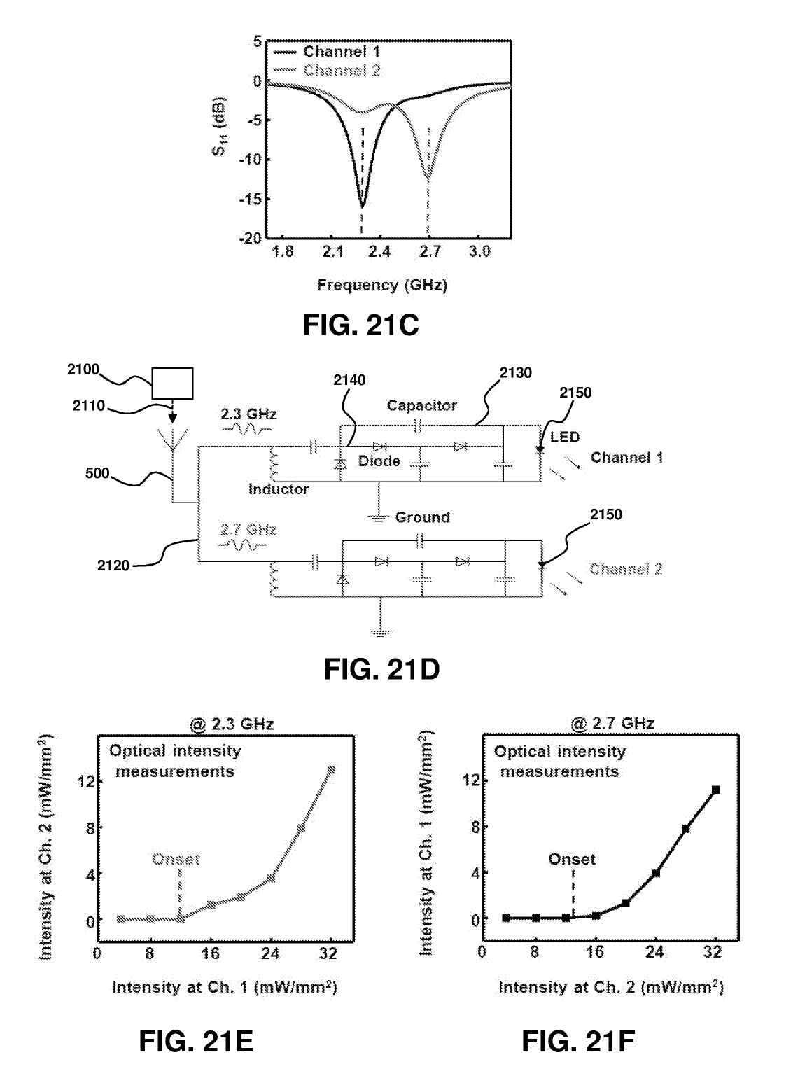

FIG. 21D schematically illustrates an externally located transmitter 2100 (not to scale) that generates a control signal 2110 that interacts with a radiofrequency harvester 2120 operably connected to active electronic components 2150, illustrated as two independently addressable LEDs. The operable connection may be via various electronic components and configurations, including impedance matching circuit 2130 and voltage multiplier 2140. In this manner, as discussed further below, provided is multiplexing capability through the use of independently addressable channels that can independently control a plurality of active electronic device components, including by channels. This is illustrated in FIG. 21D with the different frequency controls of 2.3 GHz (channel 1) and 2.7 GHz (channel 2). Accordingly, harvester antenna may be a multi-channel antenna 500. In a similar manner, a three-channel antenna 2170 provides a three different frequency control, illustrated as 2.3 GHz, 2.7 GHz and 3.2 GHz. Any type of sensor or actuator may be connected to each channel, thereby providing any number of multiplexing and multichannel configurations, depending on application of interest. For example, a plurality of actuators, such as optical light sources, thermal generators, biotherapeutic release actuators may be connected, thereby providing the ability to generate spatially varying actuation. Similarly, different types of actuators may be provided with independent control, such as to separately and independently control actuation by different means, such as optical, thermal or chemical. Similarly, a combination of actuation and sensing may be independently controlled, such as energizing optical light sources at one wavelength and sensing light of a different wavelength (e.g., excitation light to tissue at one wavelength and emission light from tissue at a different wavelength).

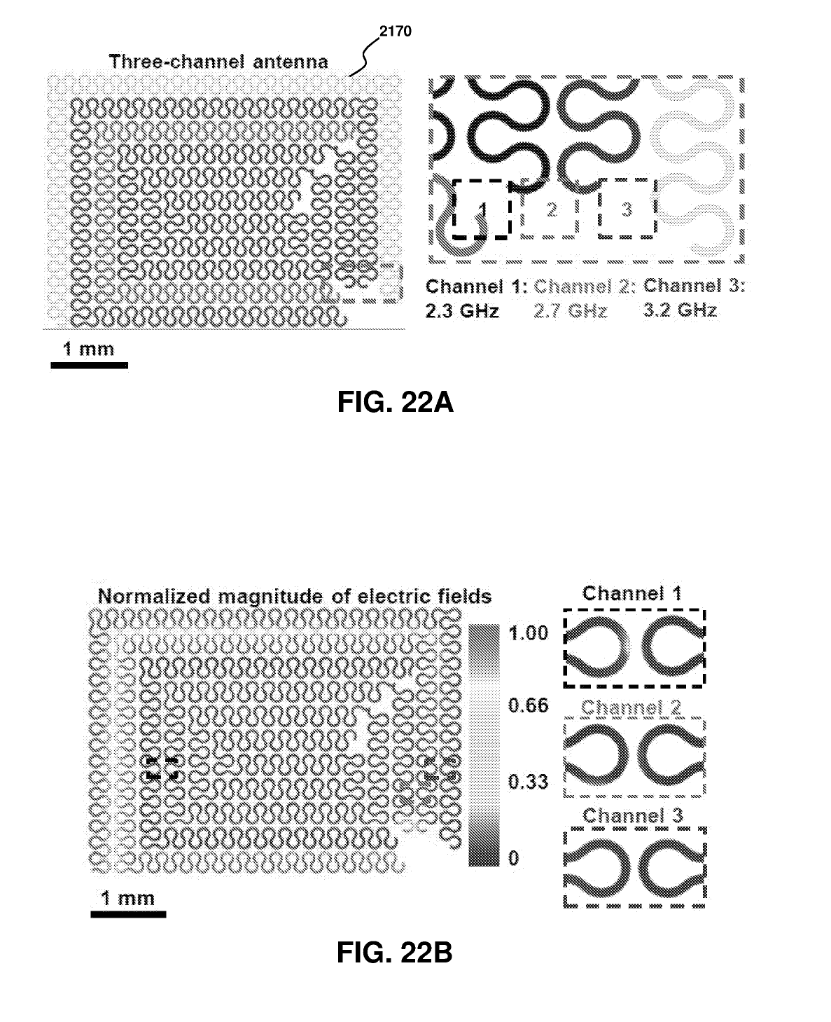

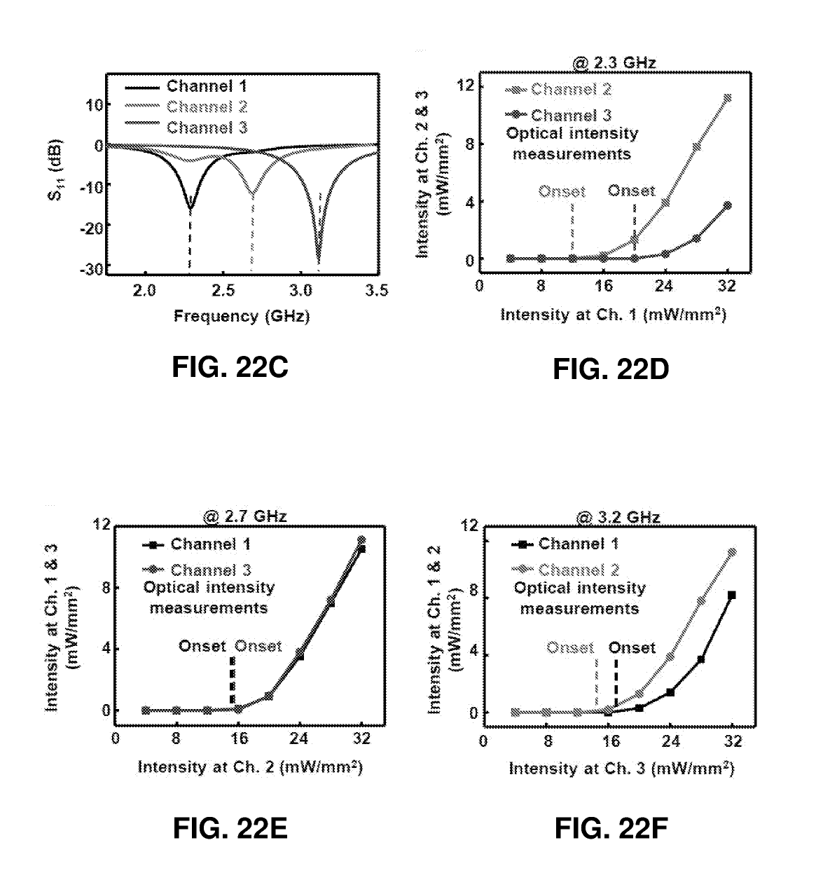

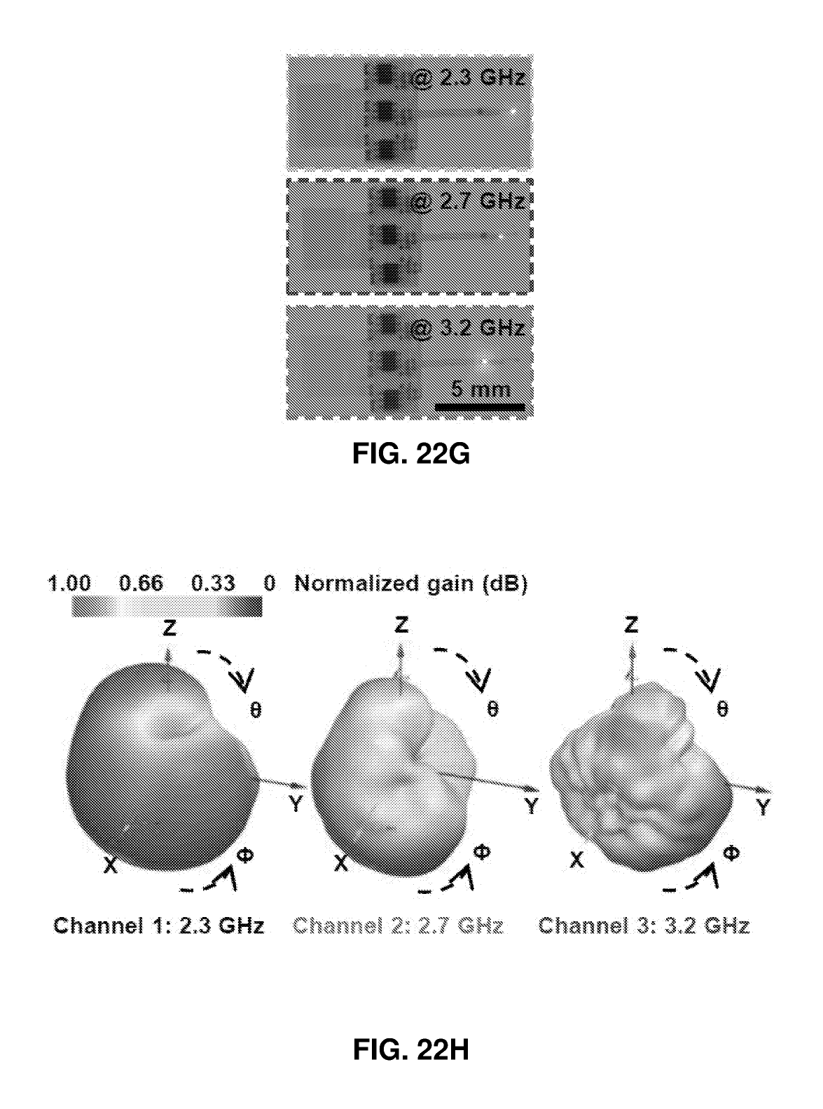

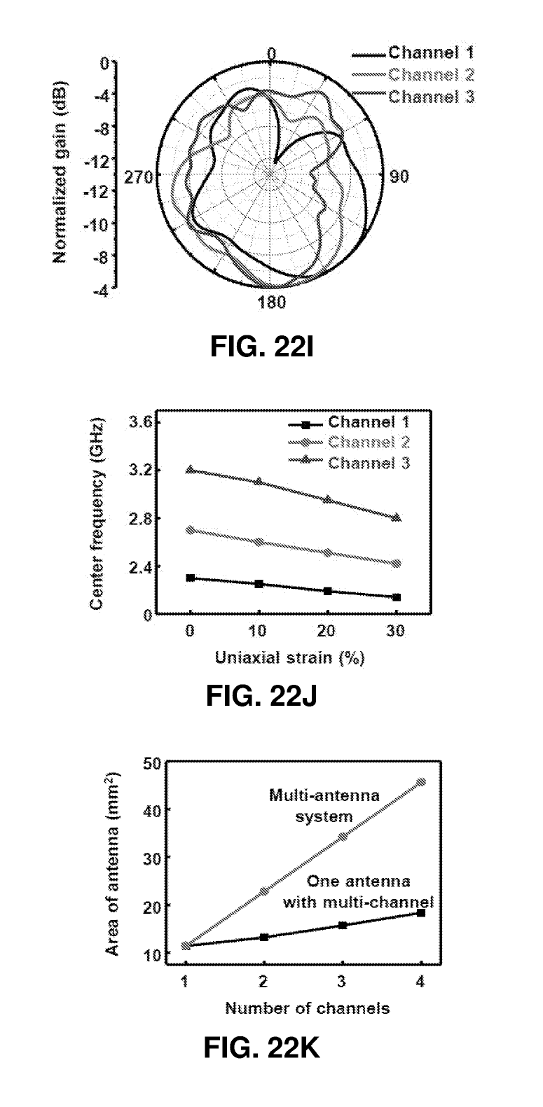

The wireless power and control system 120 may comprise a stretchable radio frequency antenna 140 with adjacent serpentine electrical conductors. FIGS. 21A-21B provide a close-up view of serpentine electrical conductors 141, with adjacent conductors 141 separated by a separation distance 142. The multi-channel antenna 500 has a plurality of capacitative coupling traces 510, with two for the two-channel antenna (FIG. 21A), three for the three-channel antenna (FIG. 22A) and n for an n-channel antenna.

The wireless power and control system may be replaced with a near field communication chip device, thereby providing additional digital control of the implantable medical device.

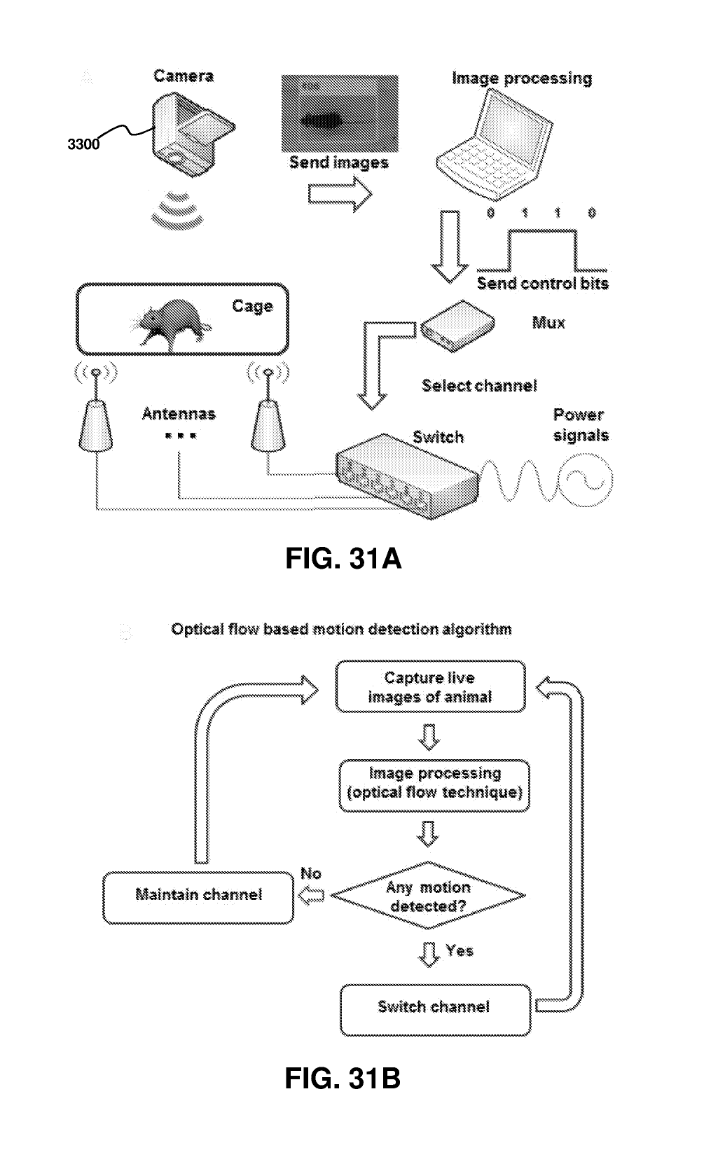

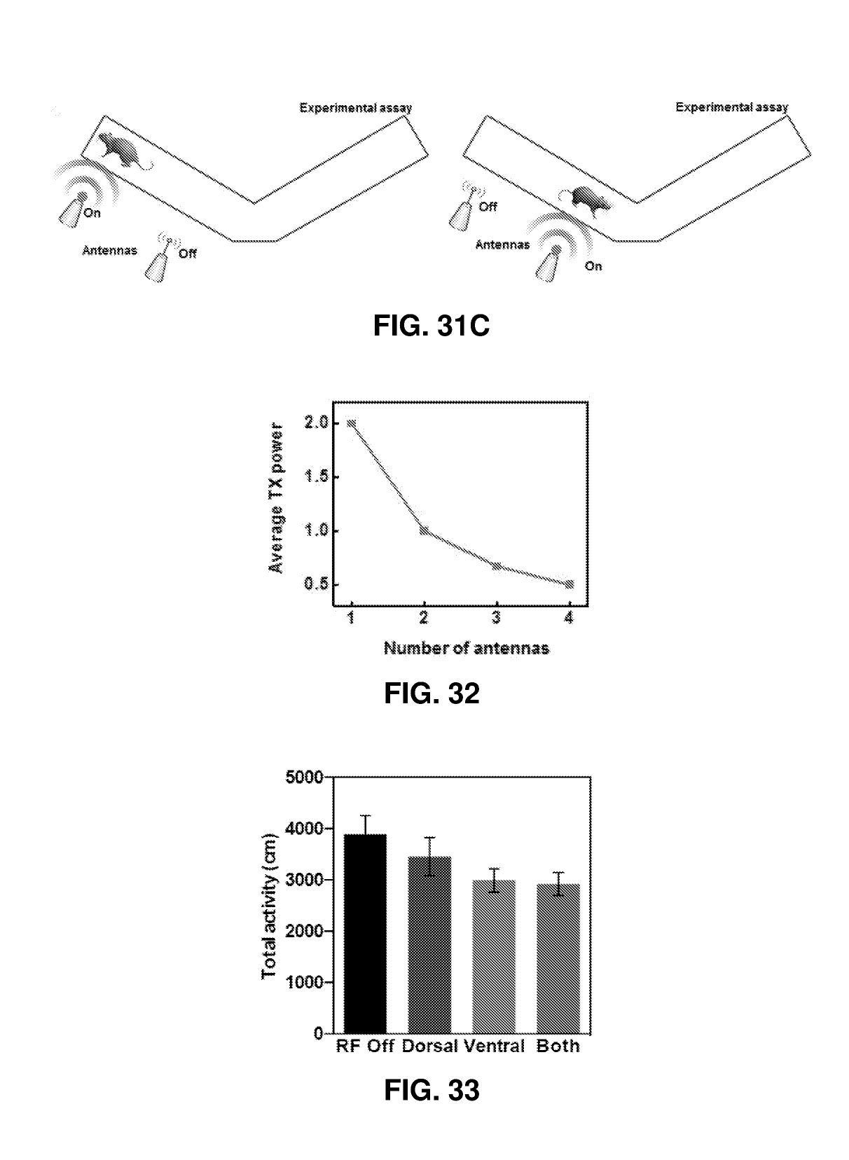

Other features of the system may include a motion tracking system 3300, including cameras and related image processing routines and controllers (FIG. 31A), a biodegradable needle 3500 (FIG. 21A).

FIG. 1B further illustrates sciatic insertion of a fully implantable soft medical device.

### EXAMPLE 1

### Fully Implantable, Soft Optoelectronics Systems for Wireless Optogenetics

The introduction of optogenetics, a technique that allows rapid and temporally specific optical control of neuronal activity via targeted expression and activation of light-sensitive proteins, has dramatically accelerated the process of mapping complex neural circuits and determining their function. Traditionally, optogenetics has required remote light sources and fiber optic delivery schemes that impose significant physical constraints on natural behaviors and thus limit utility in typical animal behavioral studies. Even recently described wireless, tether-free systems demand rigid fixtures and external components that mount to mechanically stable skeletal features (e.g. the skull) in configurations that leave fragile electrical devices vulnerable to physical damage and also limit access to non-cranial regions of the anatomy. This example provides technologies that combine soft, compliant neural interfaces with fully-implantable, stretchable wireless power and control systems to achieve chronic optogenetic modulation of nearly any region of the nervous system including the spinal cord and peripheral nerves, in freely behaving animals. Engineering design options range from stretchable appliques that interface directly with peripheral nerves to conforming filaments that insert into the narrow confines of the mouse spinal epidural space. Behavioral studies demonstrate the utility of these devices in the modulation of pain behavior, and provide evidence for their widespread use in neuroscience research and clinical applications of optogenetics outside the brain.

The use of optogenetics in the central nervous system has revolutionized the interrogation of neural circuitry by enabling temporally and spatially specific control of neuronal function. This approach utilizes light-sensitive channels, receptors or pumps to activate, inhibit, or modulate neuronal activity.^{1,2} Two commonly used optogenetic molecules are the excitatory cation channel channelrhodopsin (ChR2) and the inhibitory proton pump archaerhodopsin (Arch3.0).^{3,4} Activation or inhibition of neuronal pathways both in vitro and in vivo using these light-activated proteins has enabled detailed mapping of neuronal circuits and unprecedented insights into neuronal function.³⁻⁸

Substantial interest has developed in utilizing optogenetic modulation to dissect peripheral and spinal pathways involved in somatosensation. Furthermore, optogenetics offer the possibility of completely novel, nonpharmacologic approach to manage chronic pain and itch that afflicts millions of people.⁹⁻¹⁴ However, attempts to apply optogenetic studies to tissues beyond the brain are stymied by the inability to chronically target peripheral and spinal circuits in freely-moving animals. Studies to date have primarily focused on utilizing cumbersome tethered fiber optic cables or LED array light sources to activate opsins expressed transgenically or delivered peripherally through gene therapy approaches.^{13,15,16} Although these experimental approaches have utility, physical tethers impede movement in a way that can alter natural behaviors in ambulatory animals, frustrate natural motion in complex environments. These confounds limit the ability to manipulate neural circuits in well-established animal behavioral assays and complicate the interpretation of output data. Fiber-optic fixation requires physical bonding to a static skeletal feature of the animal, such as the skull. External fixtures have the risk of device loss due to damage by the animal itself, by a cage mate animal, or by inadvertent damage from housing or during behavioral experiments. These fibers can also damage the surrounding neural tissue during surgical insertion or during device coupling due to relative motion of the hard shaft of the fiber against soft neural tissues.^{17,18} Thin, injectable polymer filaments with integrated, cellular-scale light emitting diodes (LEDs) and externally mounted wireless power harvesting systems¹⁹⁻²² represent attractive alternatives, but do not allow for the use of optogenetics in spatially challenging and highly mobile areas like peripheral nerves or the spinal cord, which are difficult to target, but critical to the study of circuits involved in sensory input and motor output.

Chronically stable, ultraminiaturized, biocompatible devices which can safely interface with neural tissue are required for the development of innovative approaches utilizing optogenetics to better understand and treat chronic pain, itch, and other neurological disorders. Device development is challenged by difficulties in managing heat generation and power delivery, and enabling robust external control by a remote user.²³ Here, we present ultraminiaturized, soft wireless optoelectronic systems with versatile layout options that enable complete, minimally invasive implantation over multiple neural interfaces. The biocompatible devices presented here permit chronic, longitudinal experiments that would be impossible to perform with tethered optical fiber approaches or even with recently described injectable devices. We demonstrate one such application of these devices by specifically and reversibly activating both peripheral and central pain circuits to generate nociceptive responses in freely behaving, completely untethered mice.

The results presented here demonstrate that an ultraminiaturized implantable radiofrequency (RF) harvester and RF control strategies can offer versatile capabilities in optogenetics. Experimental and modeling studies establish a range of effective operating conditions for these approaches.

FIG. 1A shows a schematic view of a fully implantable, stretchable radio-frequency power/control module. The electronics include a radio frequency harvesting unit that receives signals from a separate transmitter, rectifies them, multiplies the voltages (3× multiplications) and routes the resulting direct current output to one or more μLEDs. Serpentine interconnects, miniaturized components with low modulus elastomer superstrates and substrates yield soft mechanics capable of accommodating anatomical shapes, using established concepts in stretchable electronics.

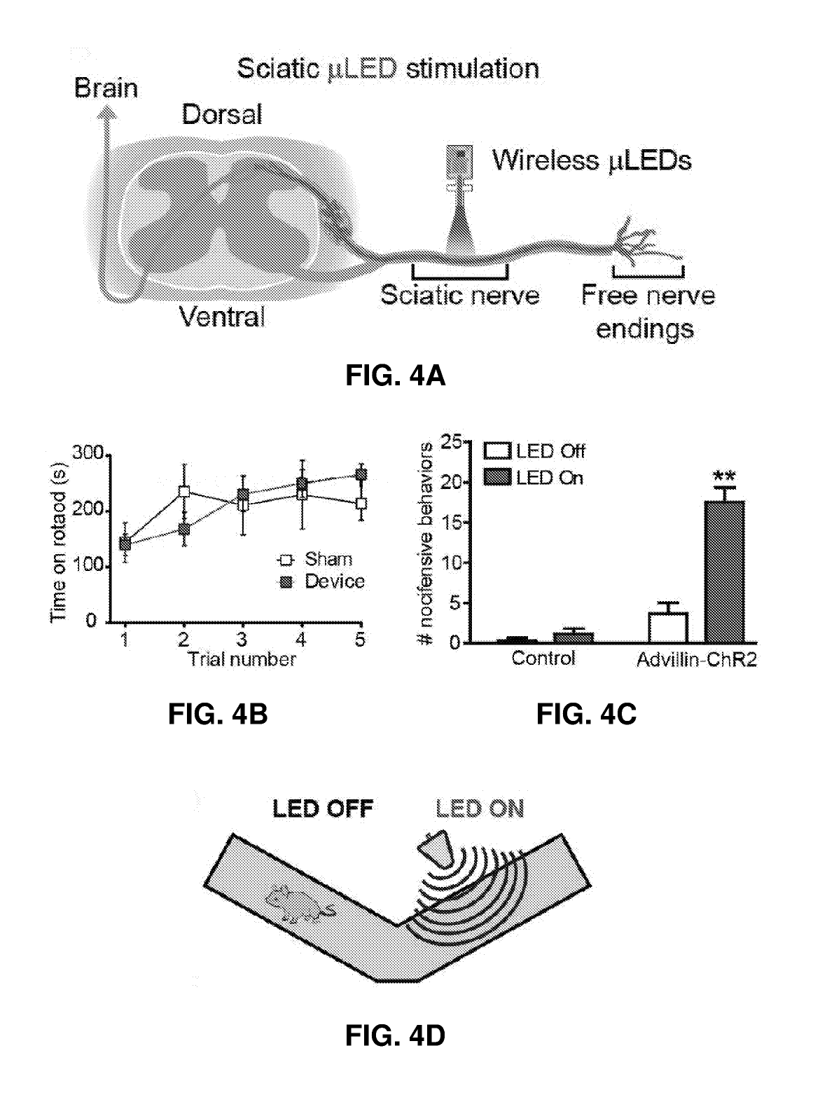

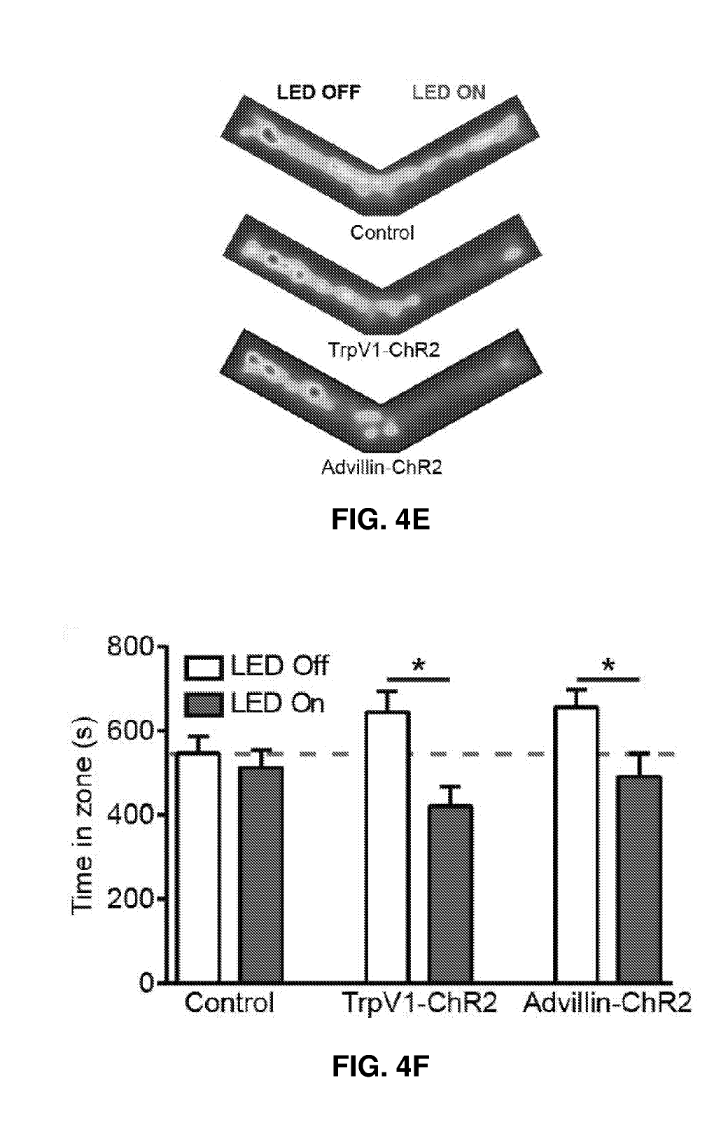

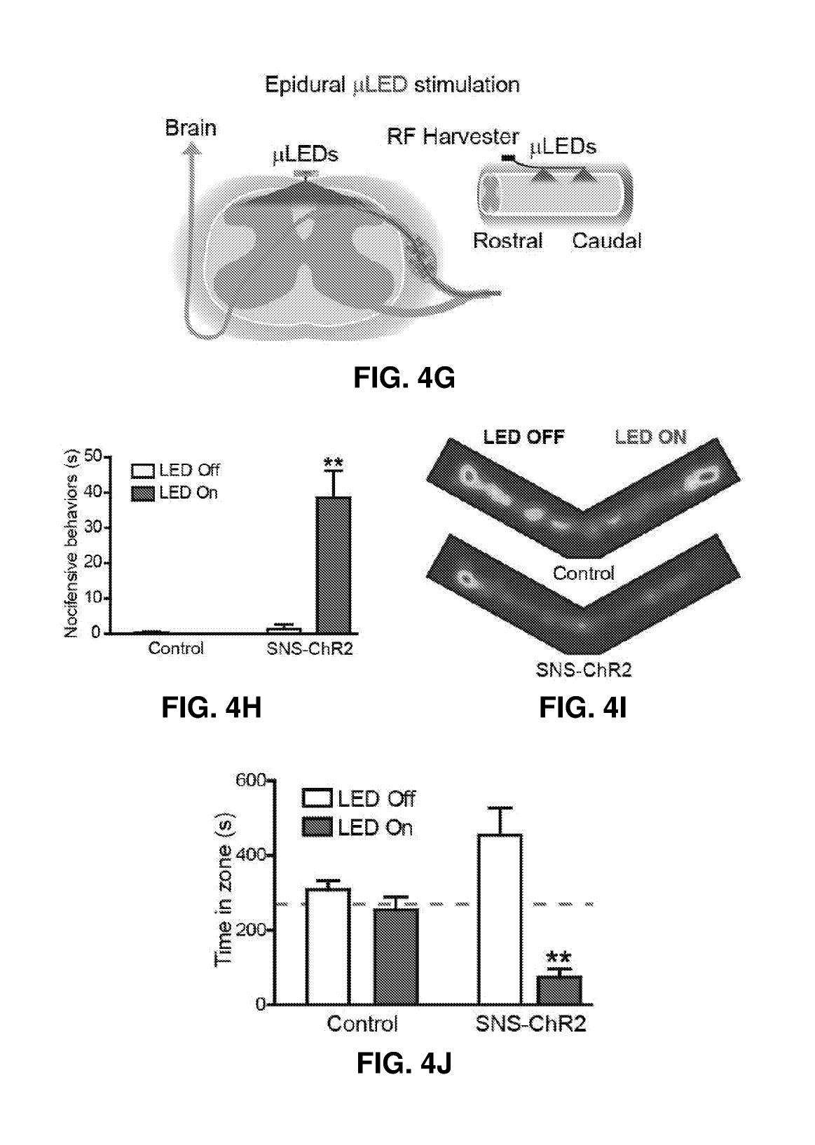

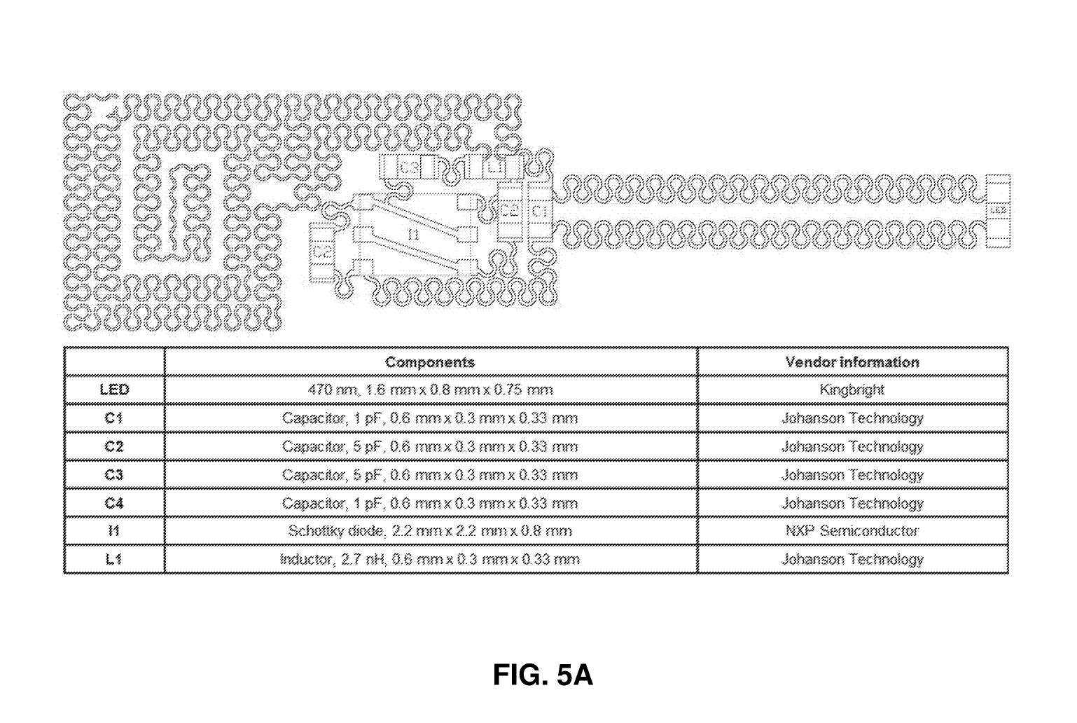

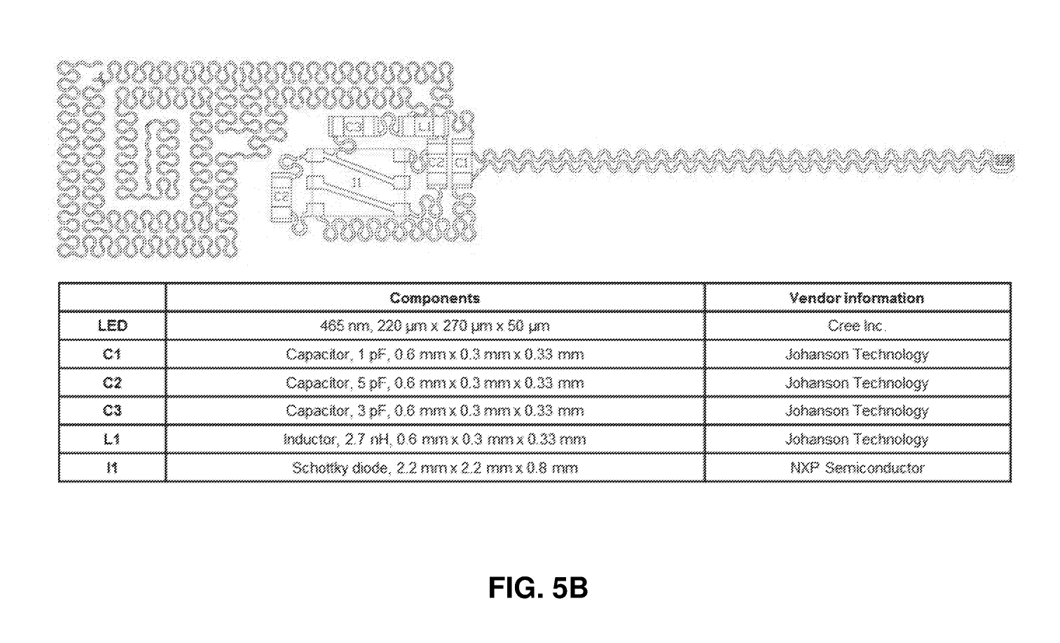

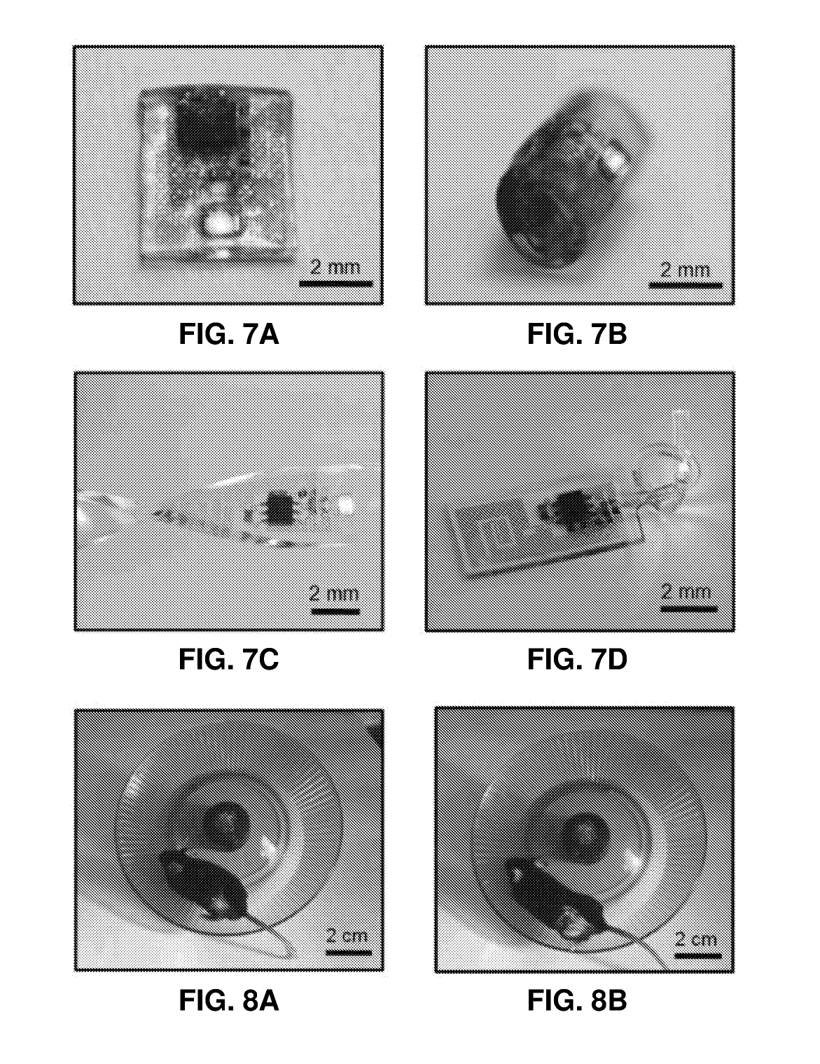

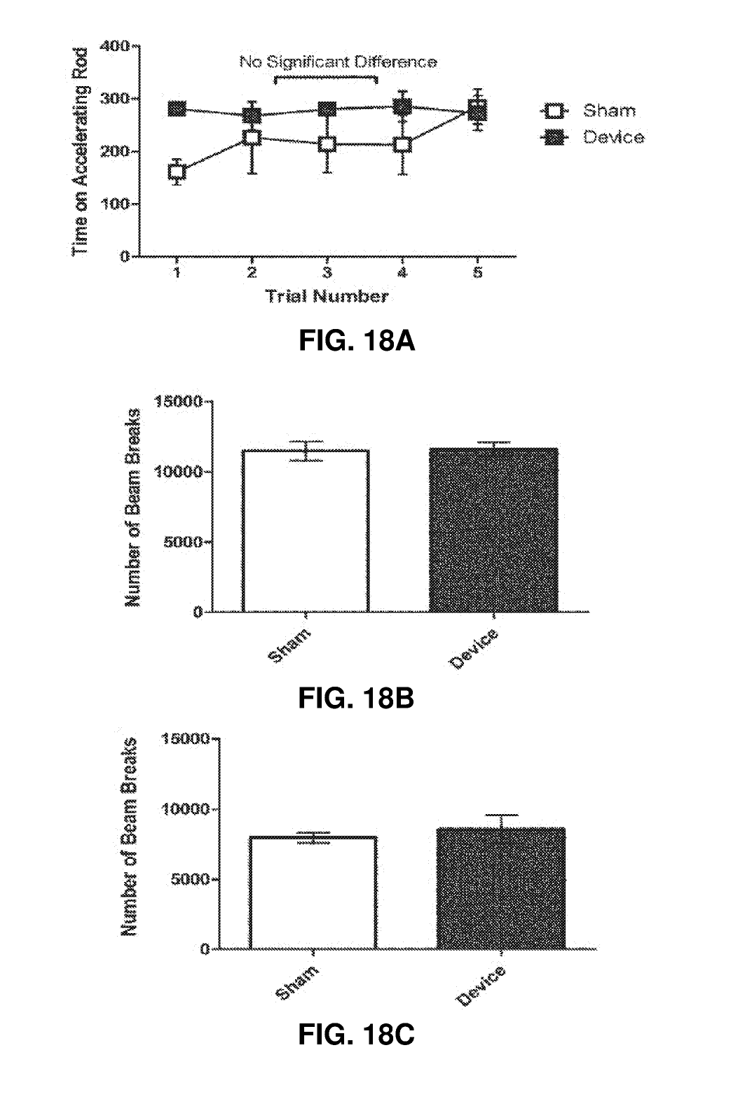

FIGS. 1B-1C illustrate the anatomy and location of the designs configured for use adjacent to a peripheral nerve embedded under muscle tissue and inserted into the epidural space for optogenetic control of the sciatic nerve and the spinal cord, respectively. The device for peripheral nerve illumination takes the form of a soft applique that slides into a muscle pocket made by the blunt dissection of the fascia, followed by folding and insertion under the gluteus maximus. Modification of the distal end encapsulating the μLED to include bilateral “wings” enables suturing to underlying muscle providing additional stabilization of the device over the nerve as shown in FIG. 1B. The epidural construction shown in FIG. 1C inserts under the vertebral bone in the epidural area, which has been exposed by thoracolumbar vertebral transition and laminectomy of the T13 spinous process. This placement centers the device over the dorsal horn of the L4-L6 spinal cord segment. The cross sectional dimensions (380 μm diameter) are suitable for implantation into the mouse epidural space. In both spinal and peripheral devices, the soft, stretchable mechanics enable stable chronic operation with minimal constraints on natural motions and behaviors of the animals (FIGS. 4B and 18A-18C). FIGS. 1D-1E provide images of devices designed for use with the sciatic nerve and the spinal cord, respectively. Serpentine interconnects in optimized configurations, together with strain isolating designs surrounded by soft elastomeric substrates and superstrates guarantee reliable operation even under extreme deformations associated with stretching, folding and twisting into knots, as in FIG. 7A-7D. These mechanical characteristics are well suited to robust operation when implanted in freely behaving animals. Images and movies of a mouse with a device interfaced to the sciatic nerve during exercise on a running wheel appear in FIG. 8A-8B. FIG. 1F shows wireless operation 6 months after implantation. The miniaturized dimensions (0.7 mm thick, 3.8 mm wide, 6 mm long) and weight (16 mg) of this system, together with its soft, compliant mechanics, renders the system nearly invisible in terms of appearance or physiological impact.

The system may be described in terms of ranges of dimensions, such as between 0.5 mm and 1 mm thick, between 1 mm and 10 mm wide, between 1 mm and 10 mm in length, and between 10 mg and 30 mg weight. Other geometries, depending on the application of interest, are available, including square, circular and ellipsoid, for example. The geometry may be described as having a footprint surface area, such as between about 10 mm² and 1 cm², and any subranges thereof.

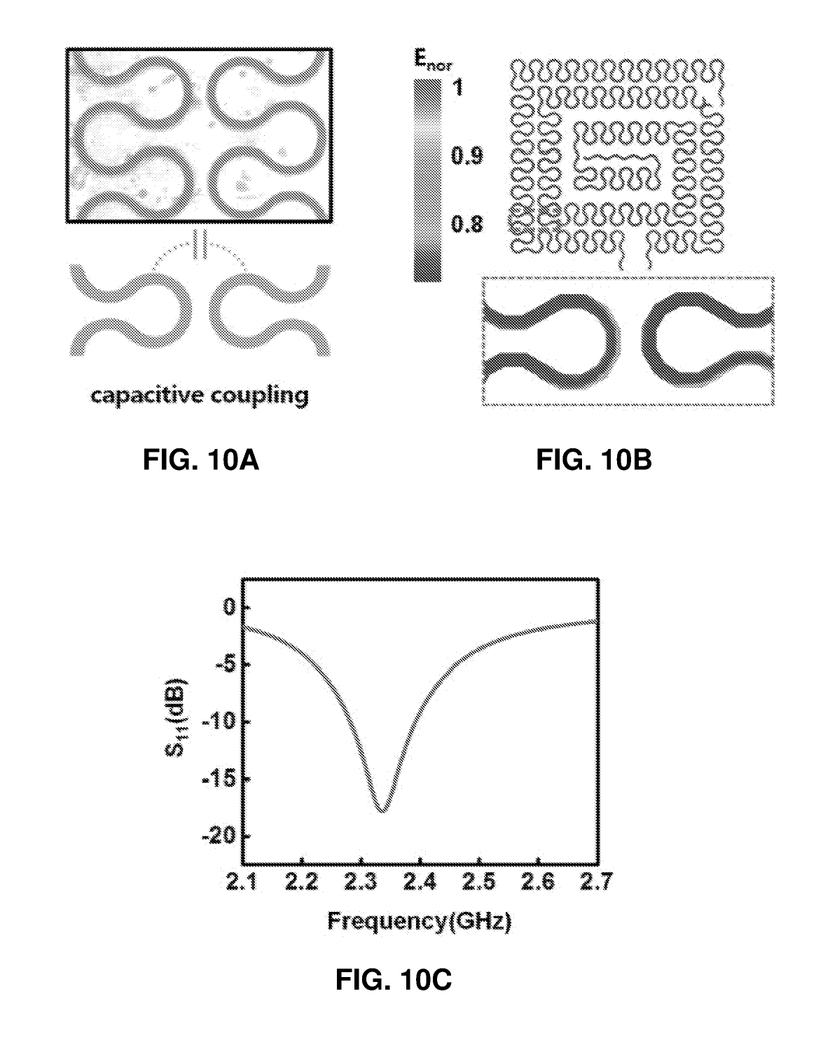

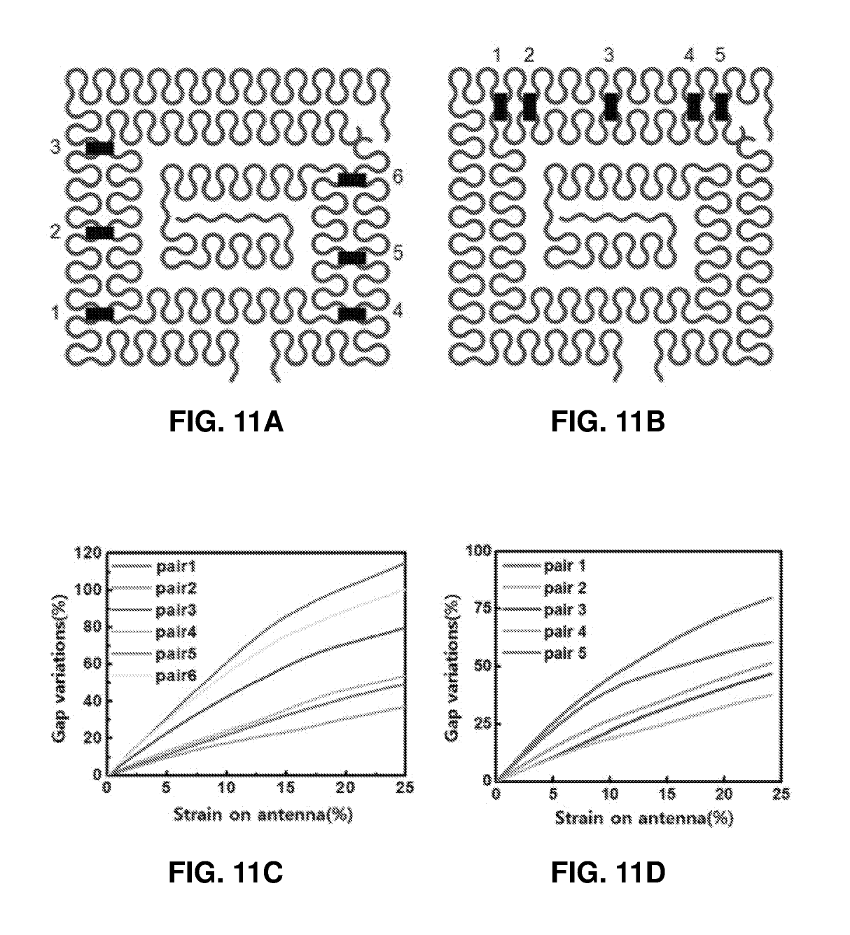

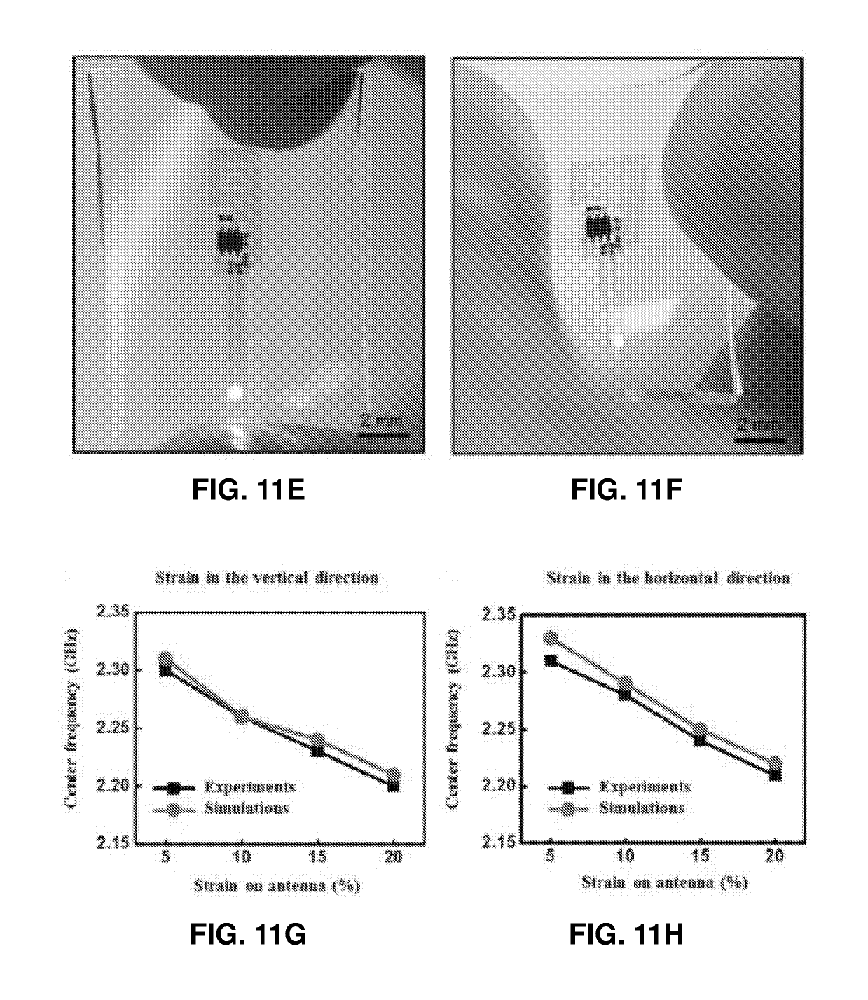

Stretchable Radio Frequency Antenna: The stretchable radio frequency antenna in the harvesting unit is an important component of the overall system. Operation relies on capacitive coupling between adjacent serpentine traces, as illustrated schematically in FIG. 10A. The overall exemplified layout offers a bandwidth of 250 MHz and a center frequency of about 2.35 GHz. The latter characteristic allows for significant size reductions compared to previously reported externally mounted, rigid harvesters that operate at 900 MHz. The former is critical for reliable operation even with variations in the center frequency that can arise from mechanical deformations, scar tissue formation, changes in temperature or hydration state. FIGS. 10B-10C show normalized electric field distributions for a representative stretchable antenna and its scattering parameter, S₁₁, respectively. Field distributions between adjacent serpentine lines in the magnified view illustrate the capacitive coupling. FIGS. 1H-1I and FIGS. 11A-11H summarize characteristics and their variations under application of strains in the vertical and horizontal directions. The coupled mechanical and electromagnetic efforts are important in design and operation. Mechanical simulations of six pairs of serpentine lines as shown in FIG. 11A reveal the effects of strain on the gaps between adjacent lines. Results in FIG. 11C show that uniaxial strains of -10% induce changes in the gaps in the orthogonal direction by up to 50%. The associated enhancements in capacitive coupling outweigh reductions in the direction of applied strain, to yield an overall shift of the center frequency toward lower frequencies, as in FIG. 1H (Right). Simulation results for strain applied in the vertical direction appear in FIG. 1I and FIGS. 11A-11H. These characteristics indicate reliable operation across the full range of strain conditions that are expected to occur during use. Related studies show that the large bandwidth can also accommodate variations in hydration over physiologically relevant ranges.

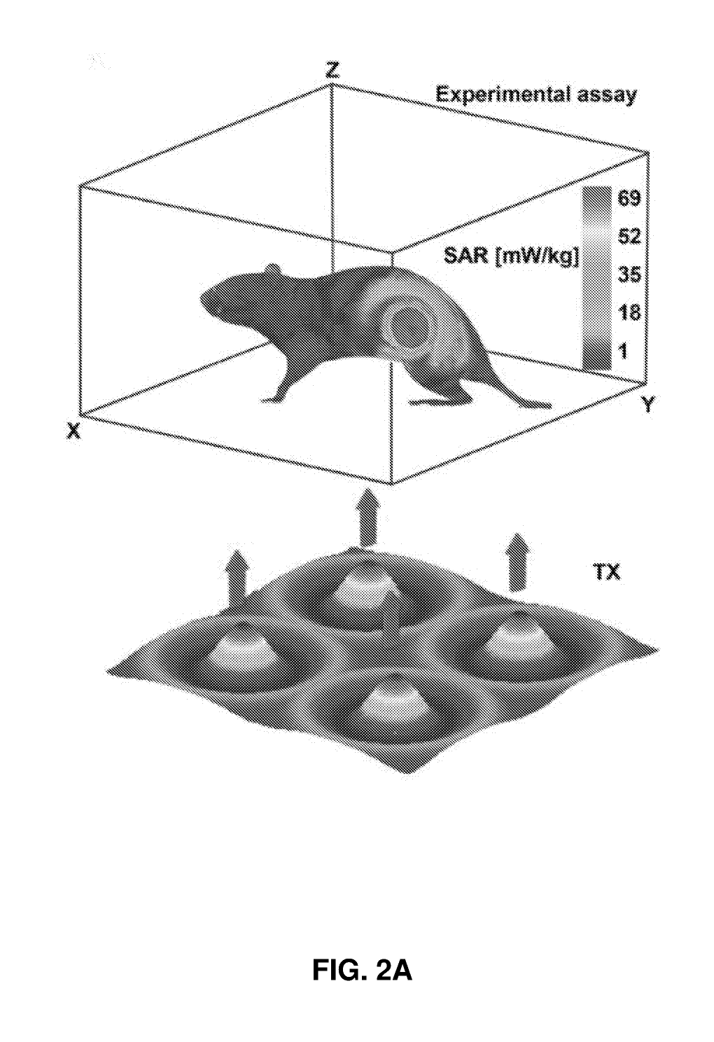

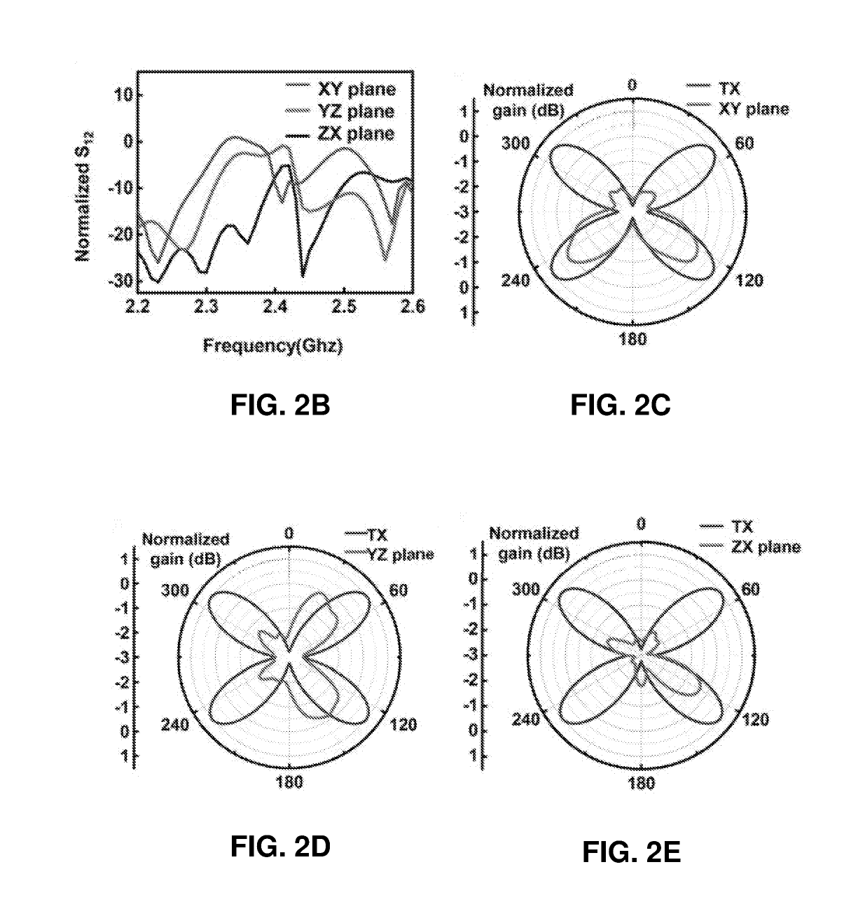

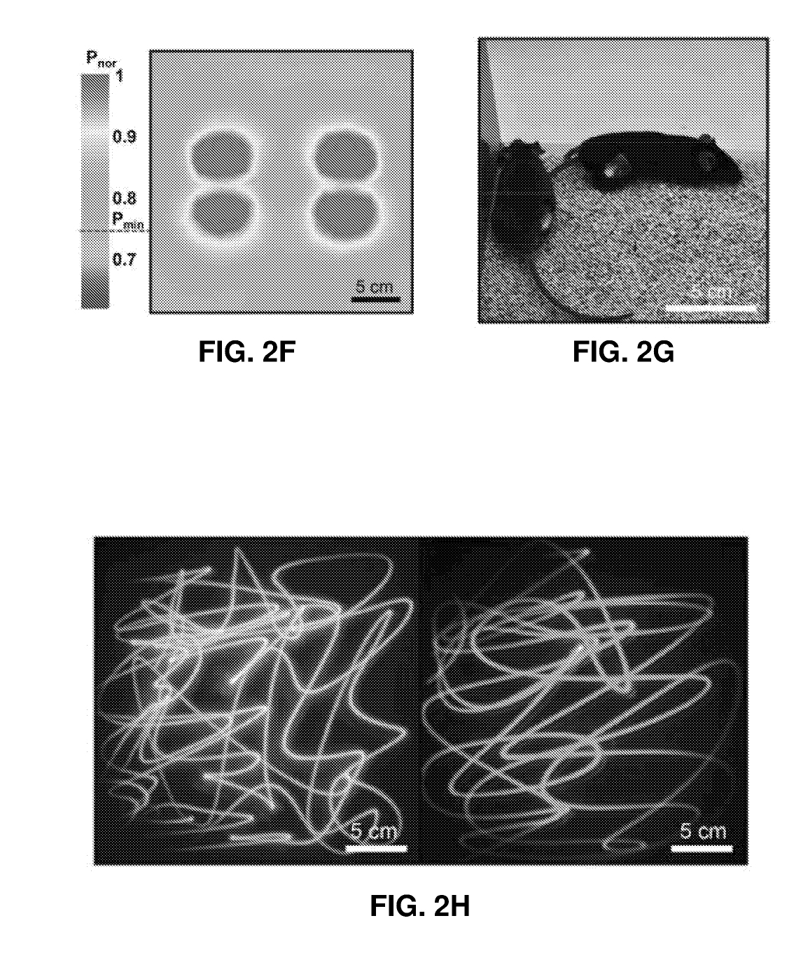

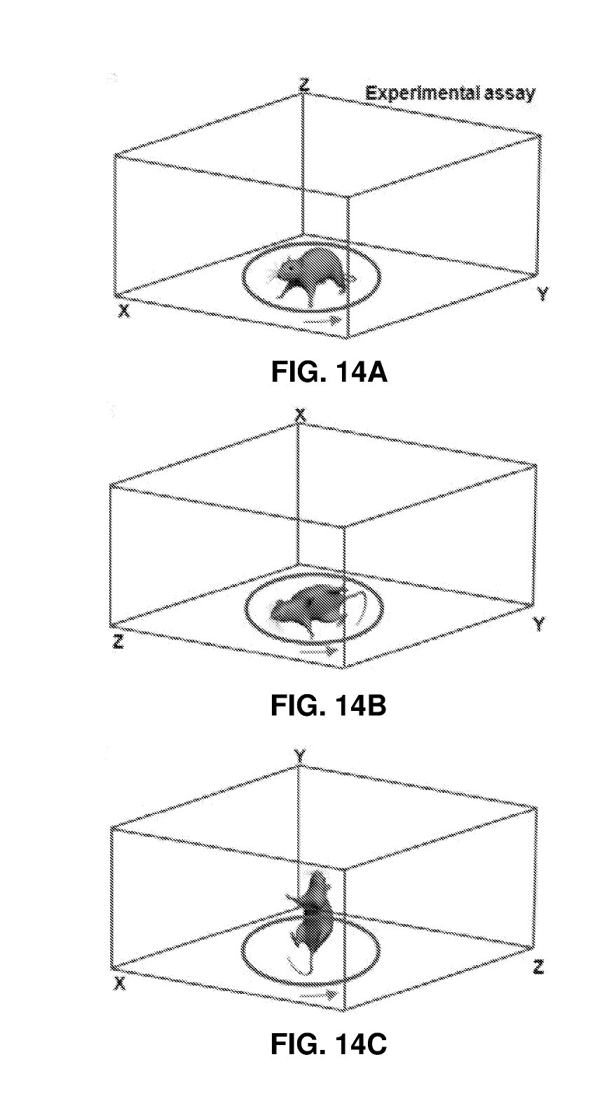

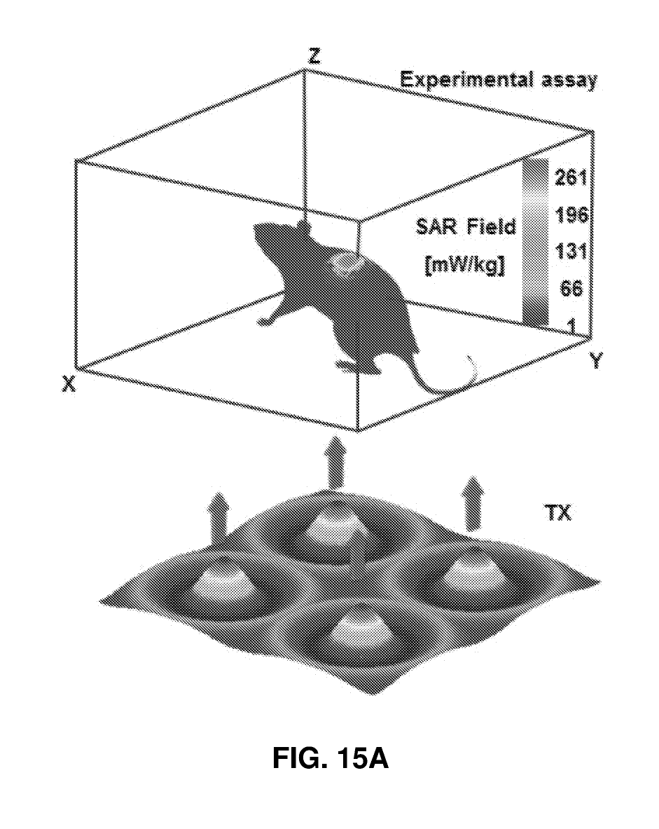

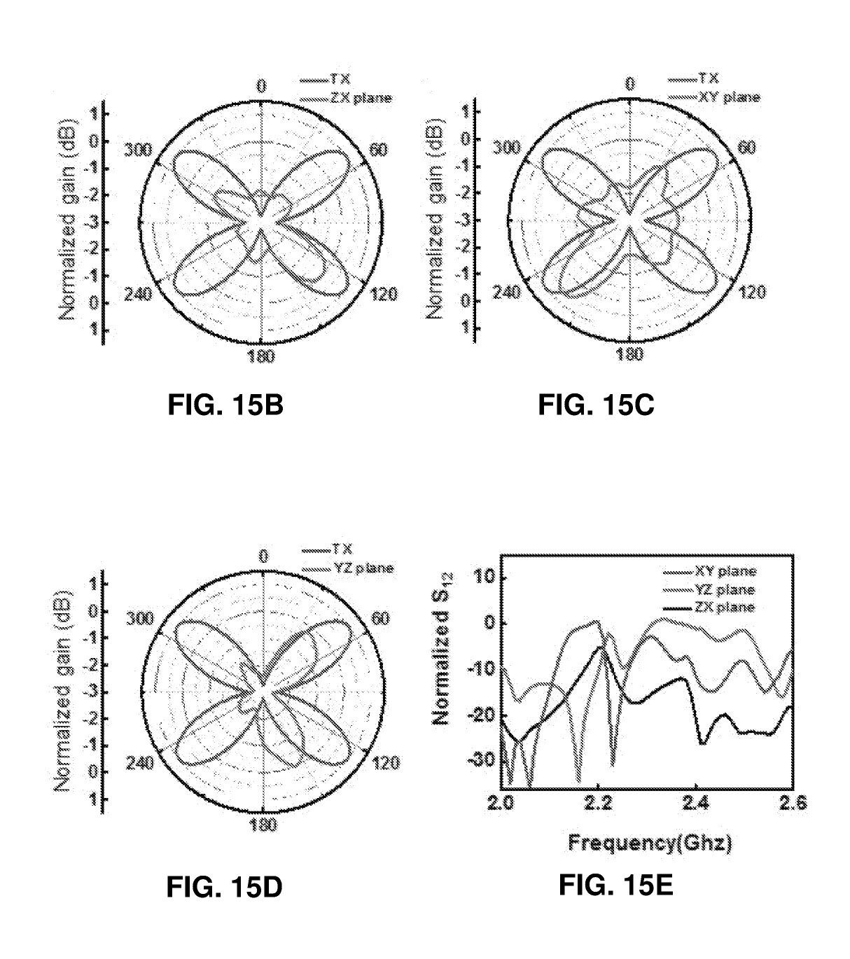

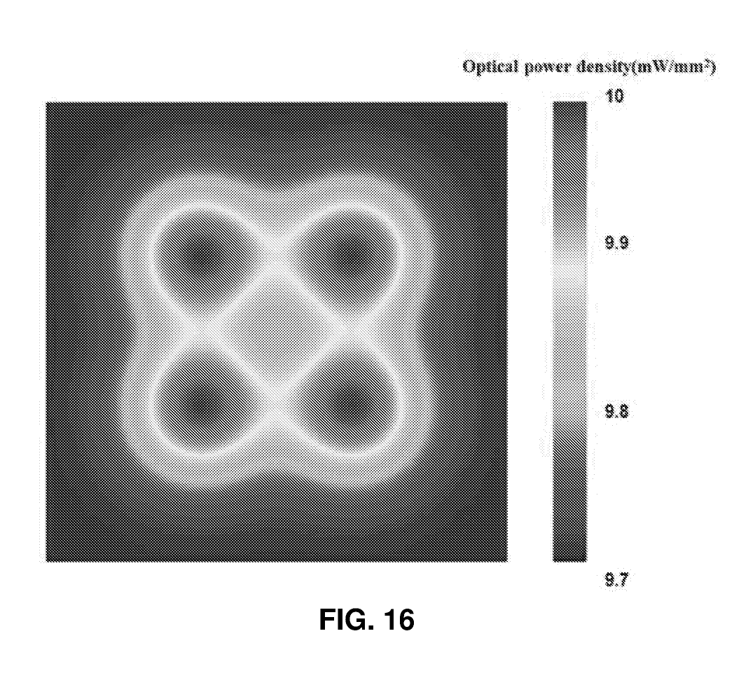

Transmission Control and Stability in Freely-Moving Mice Participating in Established Behavioral Assays: The RF transmission (TX) and control systems ensure continuous operation throughout a volume of interest (e.g. the homecage, behavioral testing arena or behavior apparatus used for the animal studies), at field strengths that lie below maximum levels determined by IEEE and FCC guidelines. FIG. 2A shows a multiple antenna configuration of four TX antennas connected to a common RF power supply. Calculated distributions of the specific absorption rate (SAR) using a mesh model for a mouse and typical RF powers (1 W) reveal that the absorption falls well below guidelines²⁴, even at the device location (sciatic nerve in this case). Experimental studies that use implantable thermal sensors are summarized below. The strength of absorption of RF by the antenna determines both the SAR values and the operation of the system. FIG. 2B summarizes the normalized transmission coefficient, S₁₂, for the cases in which the mouse (and device) is in the XY, YZ, and ZX plane for a TX system in the XY plane. The XY, YZ and ZX planes correspond approximately to the animal in walking, standing and lying postures, as illustrated in FIGS. 14A-14C. FIGS. 2C-2E show angular radiation patterns of the TX system and the device for these orientations. The XY and YZ planes yield comparable values of S₁₁ because both cases involve orthogonal orientation to the TX antennas, with similar radiation and coupling patterns. Comparatively modest coupling occurs in the ZX plane, as shown in FIG. 2E. In addition, RF from the TX antennas can be attenuated due to transmission through biological tissues.^{25,26} The intensity of the LED illumination varies in a related way. Analysis for the epidural device appears in FIGS. 15A-15E. Calculated optical power densities available in the experimental assay, shown in FIG. 16, suggest that variations of the light intensity are within acceptable ranges for optogenetic activation. Similarly, simulations of normalized electrical power suggest robust operation throughout the area, as in FIG. 2F. The photograph in FIG. 2G highlights the ability to simultaneously perform experiments on multiple animals in different orientations. Long-exposure images captured during motion of an operating device throughout an enclosure show continuous streaks of light (FIG. 2H; blue LEDs: left, green LEDs: right) demonstrating continual activation of the devices in the region of interest, providing experimental confirmation of the wireless coverage. Collectively, the results in FIGS. 1A-1I and FIGS. 2A-2H demonstrate that these devices offer robust activation in the desired biological environment at a wide variety of angles and positions.

Anatomical and Physiological Characterization of TRPV1-ChR2 and Advillin-ChR2: To test the utility of these new optoelectronic peripheral nerve- and spinal cord-targeting devices in studies of pain pathways, the ability of the devices to modulate pain-related behaviors when the light-activated cation channel ChR2 is expressed in peripheral sensory neurons is tested. First, to determine the effects of modulating activity of a broad population of sensory neurons, we crossed heterozygous Advillin-Cre mice to Ai32 mice²⁷⁻²⁹ which are homozygous for ChR2(H134R)-eYFP in the Gt(ROSA)26Sor locus, producing an Advillin-Ai32 line which expresses ChR2 in all sensory neurons. More details are provided in the methods below.

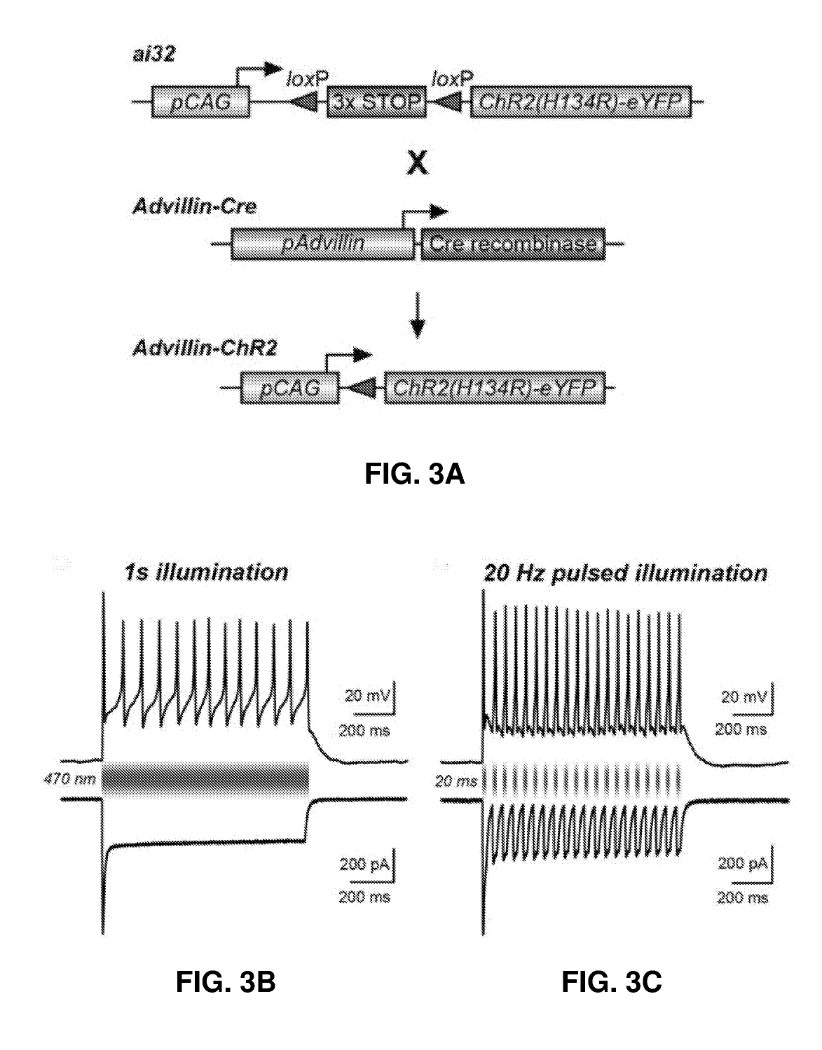

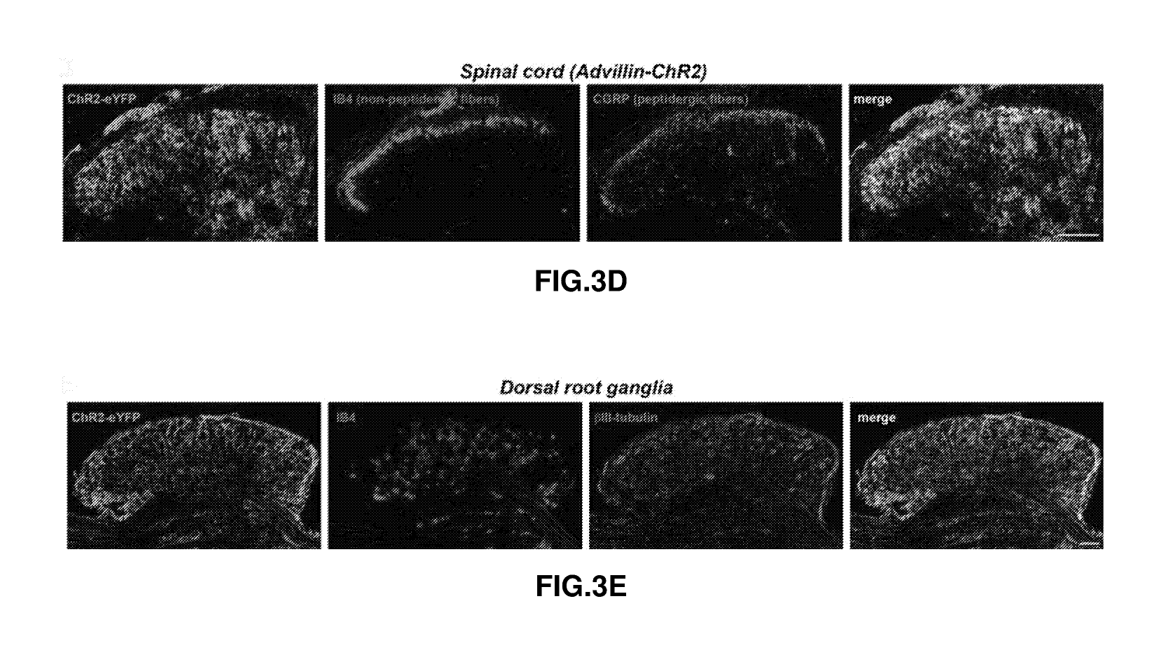

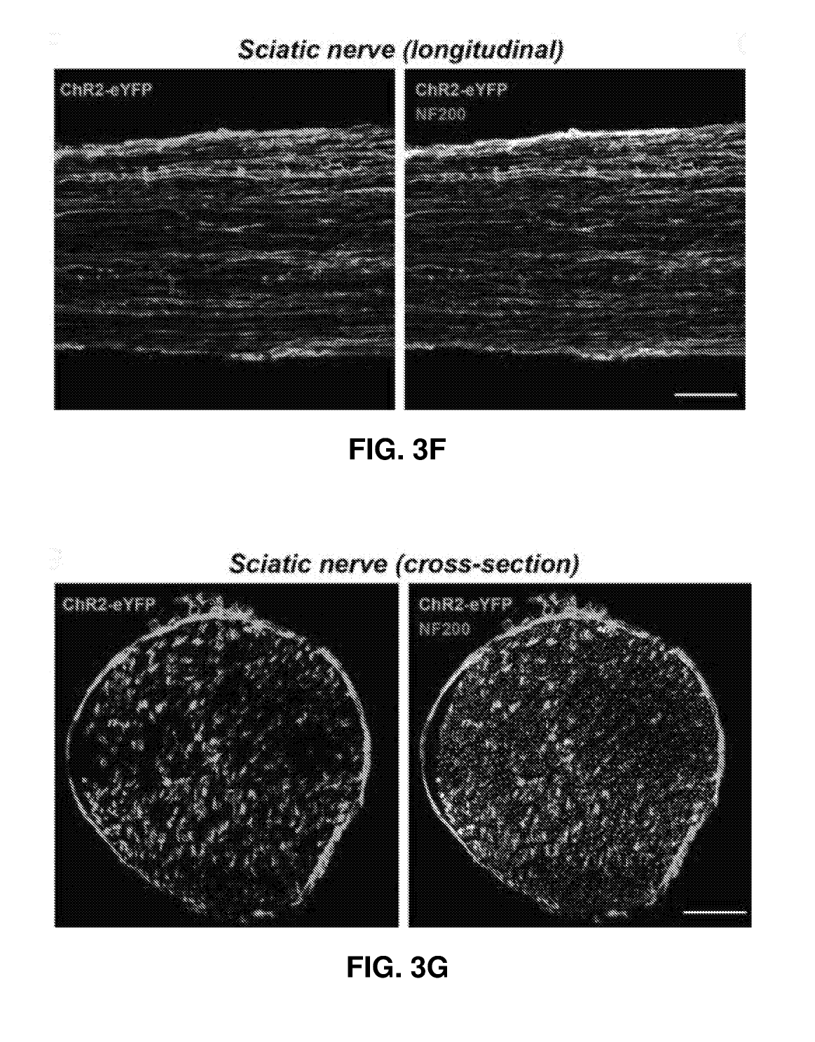

Whole-cell patch clamp recordings from cultured dorsal root ganglia (DRG) neurons from adult Advillin-Ai32 mice (hereafter referred to as Advillin-ChR2) demonstrate large inward photocurrents in response to blue light illumination (470 nm, 10 mW/mm²), confirming functional channel expression and trafficking in peripheral neurons (FIG. 3B, bottom trace). Current-clamp recordings reveal persistent action potential firing in response to constant illumination in a large number of neurons (FIG. 3B, top trace), while in others only a single action potential is elicited at the onset of illumination. Although prolonged illumination variably produces neuronal firing, we are able to drive firing with high fidelity using short pulses of light at defined frequencies up to 20 Hz (FIG. 3C), demonstrating that neuronal output in ChR2-expressing sensory neurons can be precisely controlled using blue light.

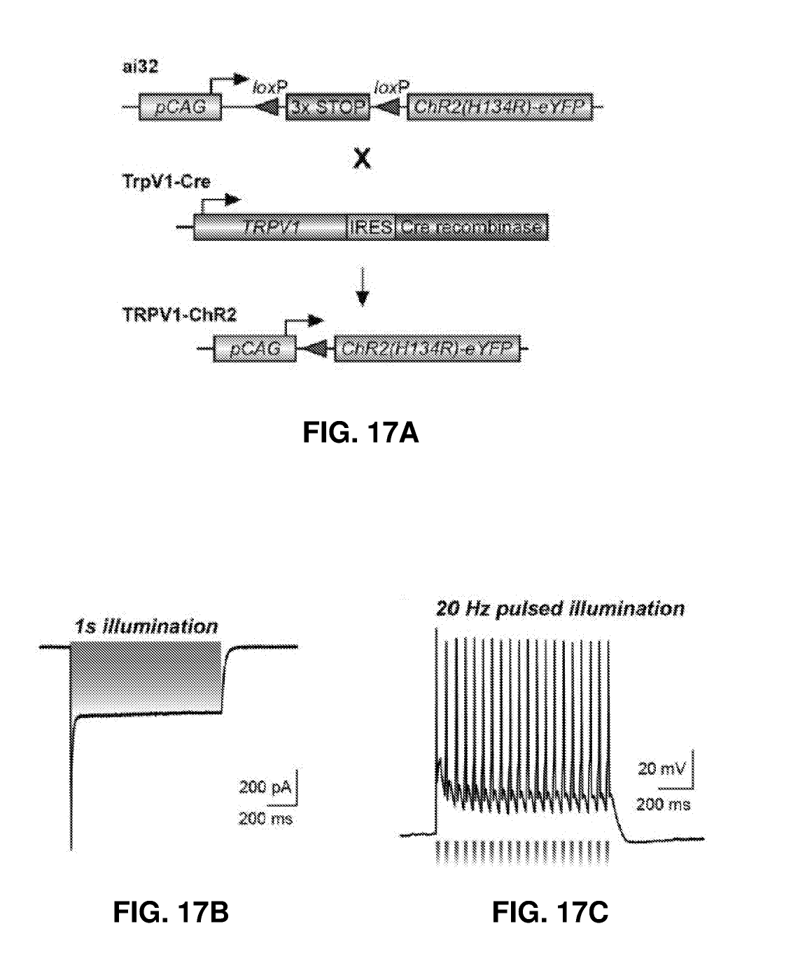

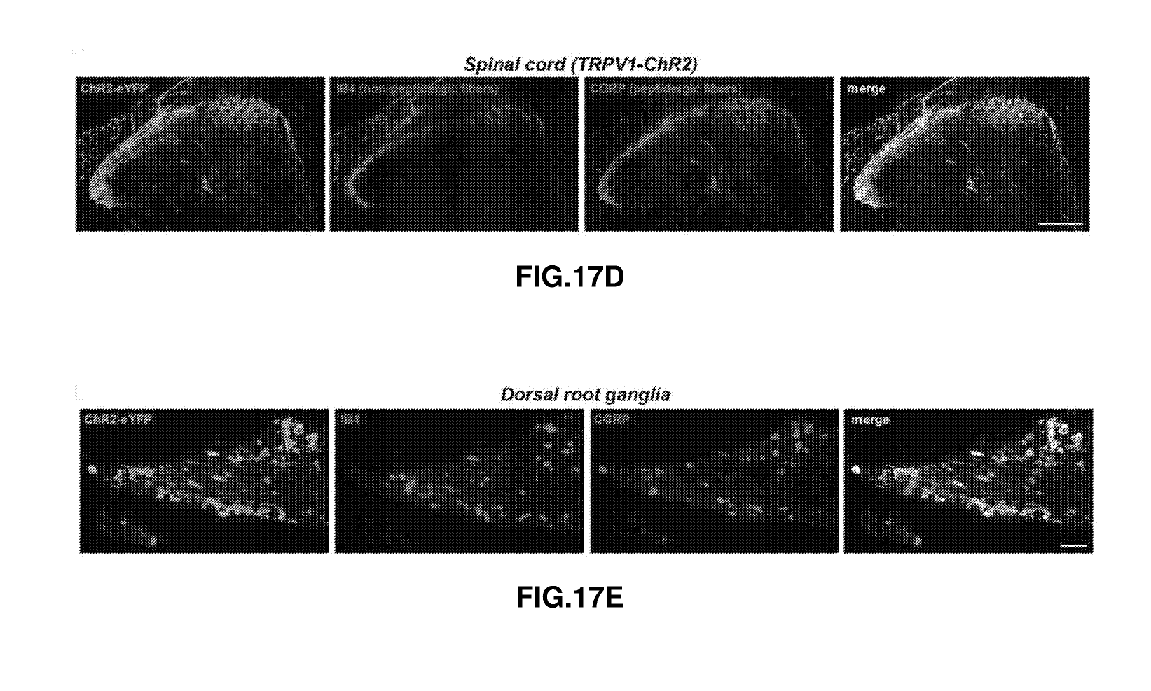

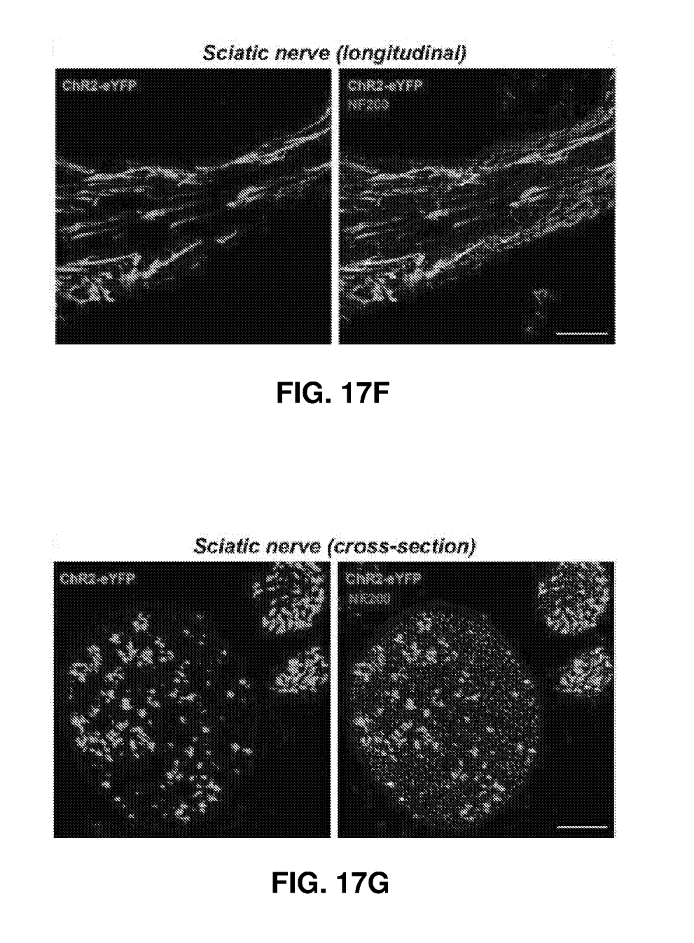

Immunohistochemical analysis of adult Advillin-ChR2 mice confirms ChR2 expression along all axes of the peripheral nervous system. Centrally projecting axons expressing ChR2 are found throughout the spinal cord dorsal horn (green, FIG. 3D), including nociceptive fibers that terminate in lamina I and II, where they are co-labeled with IB4 (non-peptidergic nociceptors, red) or CGRP (peptidergic nociceptors, purple). Staining of the dorsal root ganglion reveals ChR2 expression in most neurons, as confirmed by co-expression with the neuron-specific microtubule protein βIII-tubulin (green and purple, FIG. 3E). Additionally, all neurons that bind IB4 (red) express ChR2 (green, FIG. 3E). Also confirmed is that ChR2 is efficiently trafficked in peripherally-projecting axons. Longitudinal (FIG. 3F) and cross-sections (FIG. 3G) of the sciatic nerve shows robust ChR2 expression along the fibers, which are observed in a subset of myelinated axons marked by NF200 (purple). Similar results are observed when ChR2 expression is restricted to nociceptors by crossing heterozygous TRPV1-Cre mice³⁰ with homozygous Ai32 mice, producing a TRPV1-ChR2 line, although as expected the expression is restricted to a subset of sensory neurons (FIGS. 17A-17F).

Previous studies have shown that illumination of peripheral nerve terminals using an external light source induces spontaneous pain behavior and place aversion in mice expressing ChR2 in subsets of sensory neurons.^{15, 16} The results above indicate that in Advillin-ChR2 or TRPV1-ChR2 mice, μLED illumination of peripheral nerve fibers or of the central terminals of those fibers ending in the spinal cord should result in light-dependent induction of action potentials in sensory neurons, allowing fully wireless optogenetic control of pain-like responses in mice. Consistent with this, fiber-optic laser illumination of the exposed nerve or spinal dorsal horn in lightly anesthetized Advillin-ChR2 or TRPV1-ChR2 mice produces reflexive withdrawal behaviors (measured using EMG electrodes). With this information in hand, whether the new fully implantable, wireless μLED devices described are well tolerated in vivo, and are effective in behavioral studies, is tested.

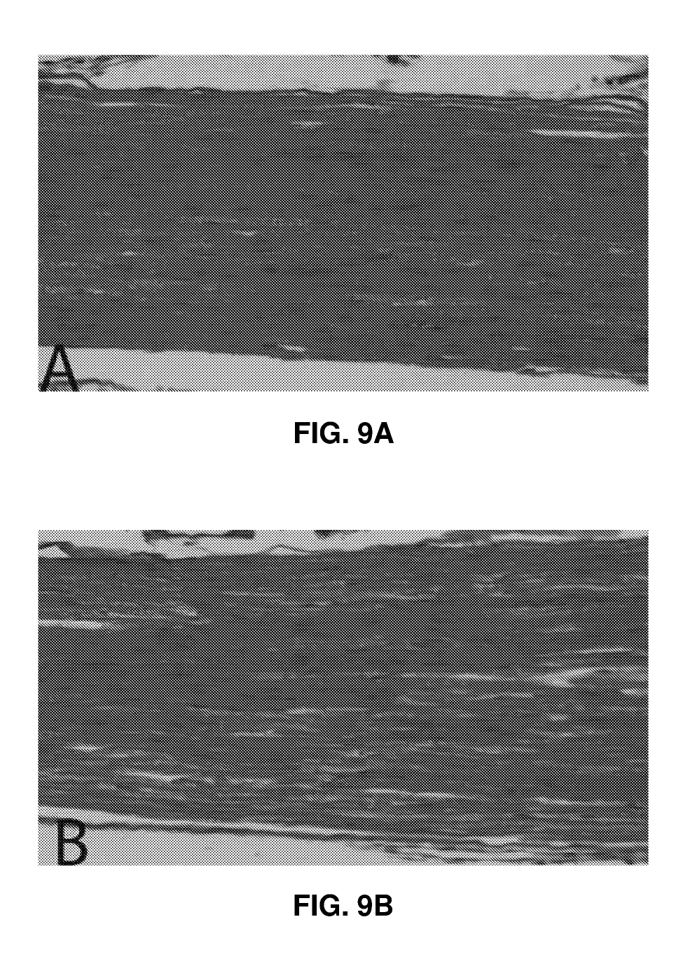

Reversible Wireless Activation of Nociceptive Pathways—Sciatic Stimulator: To demonstrate the ability of these devices to wirelessly activate sensory pathways in freely-moving mice, the sciatic optogenetic stimulator is implanted unilaterally over the sciatic nerve of Advillin-ChR2 and littermate cre-negative control mice as shown schematically in FIG. 4A and FIG. 1B. After two weeks of unilateral implantation over the sciatic nerve, the wireless sciatic optogenetic stimulator produces no significant infiltration of neutrophils, monocytes or lymphocytes; the sciatic nerve contacted with the implant is essentially indistinguishable from the contralateral sciatic nerve when assessed with hematoxylin and eosin (H&E) staining (FIGS. 9A-9B). Accordingly, any of the devices and systems provided herein may be described as “biocompatible”, in that no observable adverse immune response is as measured by this assays are detected. Furthermore, implantation of the wireless optogenetic stimulator over the sciatic nerve produces no impairment in motor behavior, even when running. Quantitative demonstration of this lack of impairment is seen in the accelerating Rotarod test and the open field test (OFT), suggesting that the implants do not affect balance, motor coordination, or locomotor activity when comparing mice with the wireless implants versus sham controls (Rotarod, p=0.894, OFT p=0.891, FIGS. 4B, 18A-18C).

Wireless powering (20 Hz, 2.35 GHz RF, 3-5 dBm) of these devices produces reversible nocifensive behaviors (flinching, hind paw licking, jumping) in Advillin-ChR2 mice (17.5 vs. 1.2 flinches, p<0.0001 vs. without illumination), but not in control mice (FIG. 4C). These spontaneous responses are consistent with nociceptive activation. To evaluate if these responses produce behavioral aversion consistent with the perception of ongoing pain (as opposed to representing reflex activation), we place mice in a modified Y-maze testing apparatus where one arm is exposed to a curtained RF (LED-ON Zone), and one arm (LED-OFF Zone) is not (FIG. 4D). Pre-testing of non-implanted devices in this arena demonstrates that activation occurs in the LED-ON Zone. Advillin-ChR2 mice demonstrate significant aversion to the LED-ON Zone (491.2 vs. 656 seconds p=0.032, FIGS. 4E-4F) compared to the LED-OFF Zone, while cre-negative control mice spend a similar amount of time in the two arms (547.0 vs. 512.1 seconds, p=0.551). Similarly, TRPV1-ChR2 mice demonstrate significant aversion to the LEDON Zone (420.5 vs. 644.5 seconds, p=0.011, FIGS. 4E-4F) compared to the LED-OFF Zone.

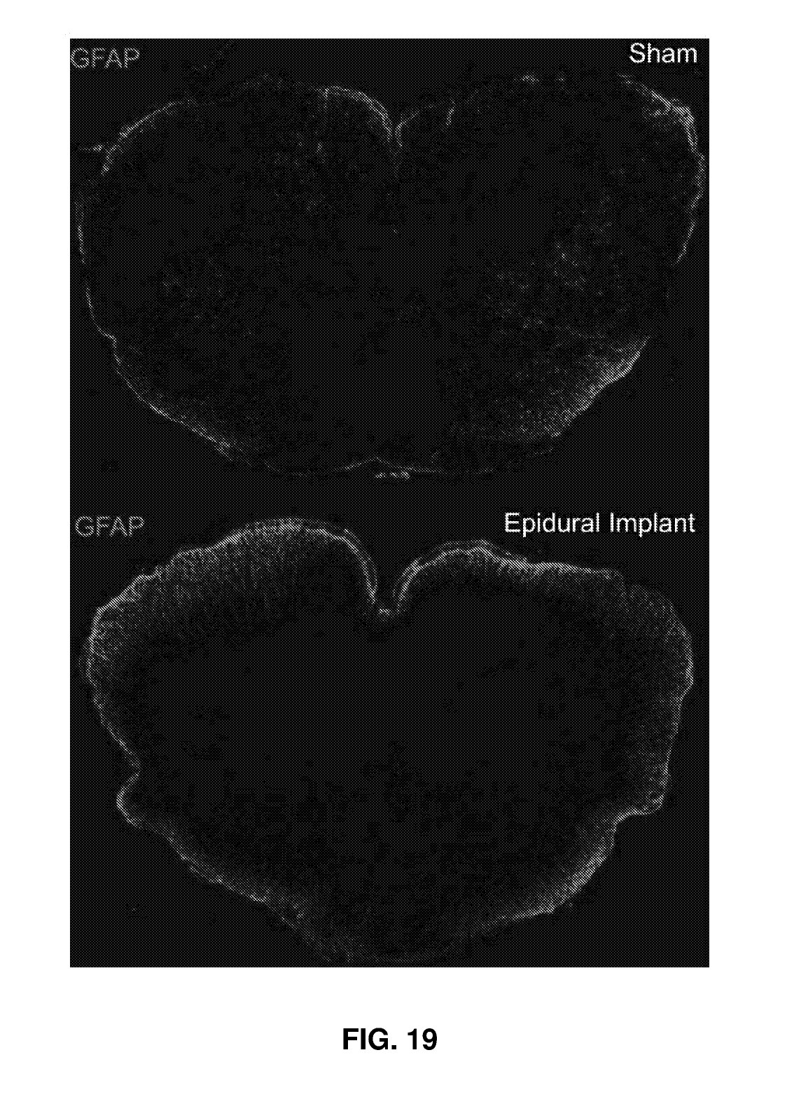

Reversible Wireless Activation of Nociceptive Pathways—Epidural Implantation for Spinal Cord Stimulation: The wireless epidural implant has potentially broader utility in optogenetic dissection of pain circuits, including primary afferent terminals innervating the spinal cord and extending to analysis of local circuitry in the spinal cord. First, we assess the impact of implantation of the spinal device in the spinal canal of wildtype C57BL/6J mice. The implantation procedure is described in detail below. Briefly, implantation consists of a small, midline dorsal incision, followed by a partial laminectomy. The soft implant is then threaded into the epidural space, allowing the stretchable antenna to rest on the dorsal vertebral process after being secured with suture (FIG. 1C). The implants produce no gross differences in glial fibrillary acidic protein (GFAP) staining (FIG. 19) in the spinal cord of mice 1 week after implantation. Furthermore, after one week of implantation, these devices produce no significant difference in motor behavior, locomotion, or coordination versus sham controls, as assessed using the accelerating Rotarod test (p=0.226) or OFT (p=0.590) (FIGS. 18A-18C).

As a demonstration of the utility of these implants to manipulate spinal circuitry, we demonstrate the ability to stimulate nociceptive afferent terminals innervating the dorsal horn of the spinal cord. Nav1.8-ChR2 mice were generated by crossing Nav1.8-Cre mice³¹ with homozygous Ai32 mice, resulting in mice in which ChR2 expression is targeted to the Nav1.8-expressing population of nociceptive sensory neurons. Nav1.8-ChR2 mice showed strong ChR2 immunoreactivity in laminae I, II and III of the dorsal horn of the spinal cord, similar to our findings in the TRPV1-ChR2 mice (FIGS. 17A-17F). Robust nocifensive responses are observed with fiber-optic illumination of nociceptor terminals in the skin, consistent with a prior report (data not shown),¹⁶ suggesting that these mice offer a suitable platform for testing the utility of the epidural devices. Upon wireless activation of epidural μLED implants(20 Hz, 2.5 GHz RF, 3-5 dBm), Nav1.8-ChR2 mice exhibit robust and reversible nocifensive behaviors that are entirely absent in cre-negative littermate controls (64.2% vs. 0% of time, p<0.001). In the real-time place aversion Y-Arm maze assay, Nav1.8-ChR2 mice with epidural implants demonstrate robust aversion to the LED-ON Zone compared to littermate cre-negative implanted control mice (73 vs. 251 seconds in LED-ON Zone, p=0.006).

The ultraminiaturized, fully implantable, soft optoelectronics systems described herein provide unmatched capabilities in a wide variety of optogenetic applications, many of which cannot be conducted with conventional fiber optic approaches or head-mounted designs. Elimination of all external hardware avoids animal interference with device operation and renders the devices imperceptible, as mice with implanted devices appear physically identical to non-implanted mice. The result enables uninhibited interactions with complex environments and with other animals. The small sizes, low effective moduli, and versatility in layout designs allow intimate interfaces to nearly any part of the body, even in locations lacking skeletal features for mechanical fixation.

Straightforward extensions of this platform allow simultaneous sensing modalities and electrical stimulation. As further described in Example 2, advanced radiofrequency techniques have the potential to allow multiplexed operation across collections of animals, in large-area environments. Thus, previously impractical studies, such as the functional interrogation of multilevel spinal circuits in the spinal cord to understand the circuits and signaling cascades that mediate normal sensory or motor function can be studied in awake, freely behaving mice. Similarly, targeting of peripheral nerves or organs which lack bony anchor points can be directly targeted using the small, fully-implantable μLED devices exemplified with our sciatic nerve stimulator design. In addition to their use for basic research, these devices may serve as important clinical tools. Since the gene therapy technology that could be used to safely deliver optogenetic channels to human cells is already in clinical testing³²⁻³⁶, these optogenetic stimulators could be adapted for use in treating chronic intractable human diseases such as chronic pain. By expanding the range of optogenetically controllable tissues with miniaturized biocompatible light sources, these devices make it possible to apply this powerful technique to a host of new approaches with basic and translational potential.

Methods: Device design and fabrication: The energy harvester comprises a stretchable antenna, impedance matching circuits, a voltage multiplier, and LEDs. The antenna, including one designed for operation at 2.3 GHz, receives RF energy from a separate source, impedance matching circuits tuned to this frequency maximize the strength of the received power, and the voltage multiplier converts this power into direct current output with voltage increased by a factor of three. Fabrication begins with a clean glass slide (75 mm long, 50 mm width, and 1 mm thickness), with a layer (200 nm thickness) of polymethyl methacrylate (PMMA, 495 PMMA A6, Microchem) and a layer (300 nm thickness) of polydimethylsiloxane (PDMS) formed by spin-casting at 3000 rpm for 30 seconds, cured at 70° C. for 6 hours. Photolithography (AZ 4620, AZ Electronic Materials) defined the necessary conducting traces in a copper film (18 μm thickness, Dupont) attached on a piece of PDMS. Solder paste (SMD290SNL250T5, Chipquik) cured at 285° C. inside a vacuum oven for 10 minutes served to electrically bond the LEDs and the SMD components to the copper traces. An encapsulating layer of PDMS, spin-cast and cured at 70° C. for 1 hour, sealed the device prior to its release from the substrate by dissolution of the PMMA in acetone. For the epidural device, the narrow serpentine area (˜380 μm width) and LED were inserted into a Teflon tube (PTFE-28-25, SAI), with an inner diameter of 380 μm. Casting and curing PDMS inside the tube followed and removal of the tube completed the fabrication.

Configuration: RF system for power transmission: The RF systems consist of a signal generator (N5181 MXF, Agilent), a power amplifier (1189/BBM3K5KKO, Richardson RFPD), a DC power supply (U8031A, Keysight Technologies) with a heat sink (53M7972, Fischer Elektronik), and TX antennas (PE51019-3, Pasternack Enterprises) with a splitter (RFLT4W0727GN, RFLambda). The amplifier and the fan connect to a separate DC power supply. The outputs (channel 1 & 2) connect to the J3 input of the amplifier, with VDD into Pin #6, 7 and GND into Pin #8, 9 and to the fan, respectively. Output of the signal generator connects to the input of the amplifier. Output of the amplifier connects to the input of the splitter, and outputs of the splitter connect to the TX antennas.

Animals and Genetic Strategy: Adult mice (8-12 weeks of age) were utilized for this study. Mice were housed in the animal facilities of the Washington University School of Medicine on a 12 hour light/dark cycle, with access ad libitum to food and water. Institutionally approved protocols were followed for all aspects of this study.

Three Cre-driver lines are used for this study including heterozygous Naᵥ1.8-Cre mice from Rohini Kuner³¹, heterozygous TRPV1-Cre mice from Mark Hoon³⁰, and heterozygous Advillin-Cre mice provided by Fan Wang.²⁷ Mice from each of these three lines are crossed to homozygous Ai32 mice from Jackson Laboratory. As previously described, Ai32 mice harbor ChR2 (H134R)-eYFP in the Gt(ROSA)26Sor locus.²⁹ To generate mice with conditional expression of ChR2 in specific populations of sensory neurons, mice expressing ChR2 from the Rosa locus (Ai32 mice) are crossed to mice expressing Cre from various sensory neuron-specific driver gene loci (Naᵥ1.8, TRPV1, or Advillin). For the purposes of this study, the three lines generated are referred to as Nav1.8-ChR2, TRPV1-ChR2, and Advillin-ChR2, respectively.

Surgical Procedure: Epidural Device Implantation: Under isofluorane anesthesia on an isothermal heating pad, a small 2 cm midline incision was made on the back, exposing the thoracolumbar vertebral transition. The paraspinal muscles were separated, exposing the T13 spinous process and lamina. A partial laminectomy was made at the rostral end of this landmark level, allowing insertion of the epidural stimulator with u-LEDs centered over the dorsal horn of the L4-L6 spinal cord segment.³⁷ The distal end of the epidural stimulator and proximal stretchable antenna were secured with 6-0 suture. The skin was closed utilizing interrupted sutures for wound integrity and mice were allowed to recover on an isothermal pad with access to food and water ad libitum.

Surgical Procedure: Sciatic Device Implantation: The surgical procedure was modified from the Chronic Constriction Injury procedure.³⁸ Mice were anesthetized with isoflurane and eyes were covered with Altalube ointment (Altaire Pharmaceuticals, Riverhead, N.Y.) to prevent corneal drying. A small skin incision was made over the greater trochanter of the femur on the left flank of the animals. The fascia connecting the biceps femoris and the gluteus maximus was blunt dissected apart to open a plane between the muscles, in which the sciatic nerve was clearly accessible. The fascia connecting the skin of underlying muscle in the area directly rostral to the incision was blunt dissected apart using needle driver forceps. The body of the device was inserted under the skin into the subcutaneous pocket generated by the blunt dissection. The gluteus maximus was pulled caudally to expose the sciatic nerve, and the tip of the device containing the μLED was folded under the gluteus and placed over the nerve. The gluteus maximus was pulled over the device and sutured into place with a resorbable Ethicon 6-0 vicryl suture (Cornelia, Ga.) to restore the original muscle architecture, and to secure the device between the muscles and above the nerve. The left flank incision was sutured closed using Ethicon 6-0 nylon monofilament suture and the mouse was allowed to recover from anesthesia in a warmed chamber.

Behavior: Spontaneous Behavior: Each mouse was placed in an individual, plexiglass behavioral chamber. Mice were allowed to acclimate for at least 30 minutes before testing in the presence of white noise generators to reduce the influence of external noise pollution on testing. To measure spontaneous behaviors, the wireless μLED devices were activated using the RF signal generator antenna at 3-5 dBm and 2.0-2.5 GHz. Behavior was recorded through an HD video camera (Sony) for one minute. Nocifensive behaviors (defined as licking hindpaw, vocalizations, or jumping) were quantified post-hoc from the video recordings.

Behavior: Real-Time Place Aversion: Real-time place aversion was tested in two arms of a Y-maze constructed of plexiglass with a layer of corn cob bedding. Each arm of the maze was 10 cm wide×100 cm long and was marked with either vertical or horizontal black stripes with a neutral area between the arms. To generate the RF signal, one antenna was located below an arm of the maze allowing for the control of μLED devices through the maze floor and a second antenna was positioned on the side of the same arm to ensure complete local field coverage. To begin the experimental protocol, a mouse was placed in the neutral area of the maze and was continuously monitored and recorded through a video connection for 20 minutes. During this time an experimenter blinded to the genotype manually controlled the RF signal by watching the monitoring system. Upon entry of the mouse into the “ON” chamber, activation of the μLED device through the RF antenna was initiated; likewise, upon departure from the “ON” chamber RF activation was terminated. Video data were collected and time-in-chamber was analyzed using Ethovision software (Noldus, Leesburg, Va.).

Behavior: Rotarod

The method for this testing has been described previously.^{39,40} An accelerating Rotarod (Ugo Basile) was utilized to study motor coordination and balance after implantation of the epidural optogenetic stimulator and the sciatic stimulator. Five consecutive acceleration trials were performed with 5 minute breaks separating each acceleration trial.

Behavior: Open Field: As described previously, locomotion was measured in a Versamax Animal Activity Monitoring System (AccuScan Instruments) open field arena.^{39, 40} Mice were initially habituated to the climate-controlled test room 1 hour before testing. Locomotor activity was assessed by recording beam breaks in this 42 (length)×42 (width)×30 (height) cm chamber for 1 hour. The total distance traveled during this time, time spent moving, and the number of horizontal beam breaks was calculated for the entire chamber.

Electrophysiology: Whole-cell patch clamp recordings were made from cultured DRG neurons using pipettes with resistance values ranging from 2-3 megaohms, when filled with (in mM) 120 potassium gluconate, 5 NaCl, 2 MgCl₂, 0.1 CaCl₂, 10 HEPES, 1.1 EGTA, 4 Na₂ATP, 0.4 Na₂GTP, 15 sodium phosphocreatine; pH adjusted to 7.3 using KOH, osmolarity 291 mOsm. The extracellular solution consisted of (in mM): 145 NaCl₂, 3 KCl, 2 CaCl₂, 1.2 MgCl2, 10 HEPES, 7 glucose; pH adjusted to 7.3 with NaOH. Recordings and light stimulation were performed using Patchmaster software (HEKA Instruments, Bellmore, N.Y.) controlling an EPC10 amplifier (HEKA Instruments). Neurons were optically stimulated with collimated light through the microscope objective, using a custom set-up with a blue LED (M470L2; Thorlabs) coupled to the back fluorescent port of an Olympus BX-51 microscope. Light intensity at the focal plane (10 mW/mm²) was calculated using a photodiode (S120C, Thorlabs) and power meter (PM100D, Thorlabs).

Immunohistochemistry: Mice were deeply anesthetized with a ketamine, xylazine, and acepromazine cocktail, then transcardially perfused with cold 4% paraformaldehyde in PBS. Lumbar DRG, spinal cord, and sciatic nerves were dissected and placed in 30% sucrose in PBS for overnight cryopreservation, then frozen in OCT. Frozen tissue was then sectioned in a −20° C. cryostat (Leica) at either 30 μm (spinal cord and sciatic nerve) or 18 μm (DRG) directly onto frosted glass slides. IHC was conducted as described previously.⁴¹ Goat anti-CGRP (1:400, AbD Serotec Cat #1720-9007), rabbit anti-GFP (1:1000, Molecular Probes (Invitrogen) Cat #A11122), mouse anti-NF200 (1:400, Millipore Cat #MAB5266), mouse anti-GFAP (1:500, Cell Signaling Technologies), and mouse anti-βIII-tubulin (1:1000, Covance Research Products Inc Cat #PRB-435P-100) were utilized while IB4+ labeling was obtained using an Alexa Fluor 568-conjugated IB4 (1:400, Invitrogen Cat #121412). Research Resource IDs are provided below to assist the reader. Fluorescent-conjugated secondary antibodies (Life Technologies) were used to visualize primary immunostaining were: donkey anti-goat AF647 (1:500), donkey anti-rabbit AF488 (1:500), and goat antimouse AF647 (1:500). Slides were sealed overnight with Prolong Gold Antifade Mountant with DAPI (Life Technologies). Images from sealed slides were obtained using a Leica SPE confocal microscope, with gain and exposure time constant throughout image groups.

| | | | | Research |
|---|---|---|---|---|
| Antibody | Dilution | Company | Catalog ID | Resource ID |
| Goat | 1:400 | AbD Serotec | 1720-9007 | AB_2290729 |
| anti-CGRP | | | | |
| Rabbit | 1:1000 | Molecular | A11122 | AB_22156 |
| anti-GFP | | Probes | | |
| | | (Invitrogen) | | |
| Mouse | 1:400 | Sigma Aldrich | N0142 | AB_2149763 |
| anti-NF200 | | | | |
| Mouse | 1:1000 | Millipore | 05-166 | AB_291637 |
| anti-βIII- | | | | |
| tubulin | | | | |
| Mouse | 1:500 | Cell | 3670 | AB_561049 |
| anti-GFAP | | Signaling | | |
| | | Technologies | | |

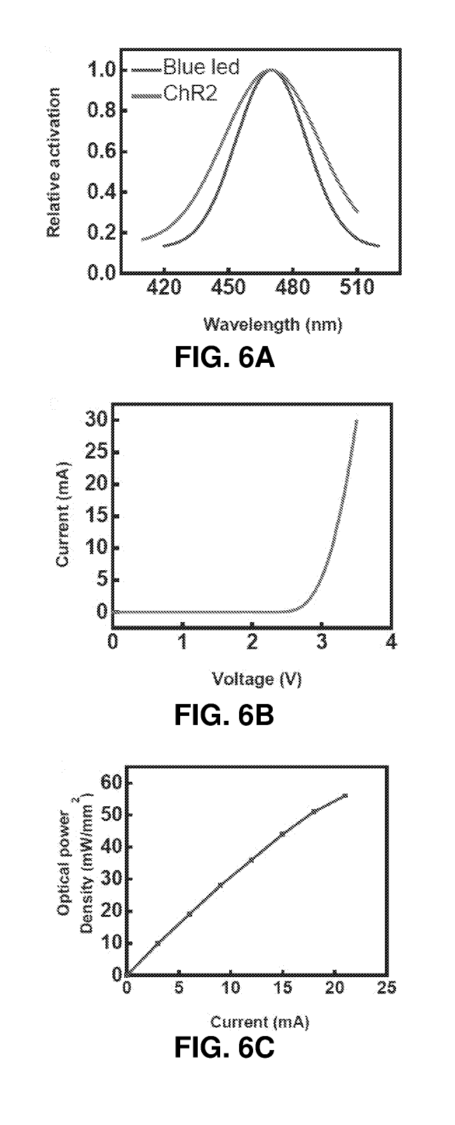

Characteristics of LEDs: FIGS. 6A-6C show characteristics of the LEDs. The built-in voltage is 2.9V and the peak emission wavelength is 470 nm, matched to the sensitivity of the channelrhodopsin. The current-voltage graph shows current requirements of 5 mA at 3.0 V and 30 mA at 3.5 V. The LED has a radiation angle of 120°.

Physiology: Mice were perfusion fixed with 4% paraformaldehyde. The portion of the sciatic nerve underlying the device was dissected, fixed overnight in 4% paraformaldehyde, and embedded in paraffin. The nerve was cut in 6 μm longitudinal sections and stained with hematoxylin and eosin. These suggest that implantation of the device does not alter the microscopic structure of the sciatic nerve or induce inflammation in the nerve as shown in FIGS. 9A-9B.

Center frequencies dependency on hydration: We performed simulations to investigate characteristics of the stretchable antenna how hydration affects performance, especially a center frequency. In this calculations, we used Colecole relaxation dielectric model and parameters for dry and wet skin described.

$${ɛ}_{r} ( ω ) = {ɛ}_{a} + {\sum }_{n} \frac{{Δɛ}_{x}}{1 + {( iw {τ}_{x} )}^{( 1 - {α}_{n} )}} + \frac{{δ}_{i}}{iw {ɛ}_{0}} ,$$

where δ is conductivity, τ is the relaxation time constant, ε₀ is the static relative permittivity, εᵣ and is the relative permittivity.

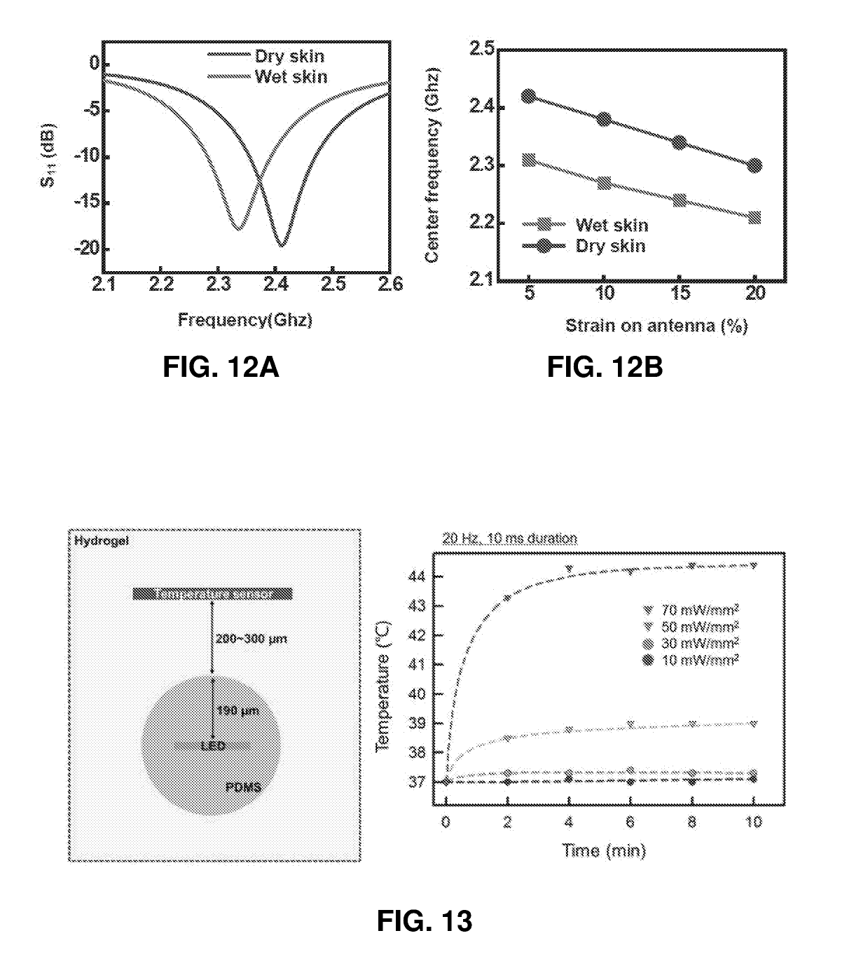

Simulations results in FIG. 12A reveal that a center frequency of the stretchable antenna when implanted under wet skin is shifted toward lower frequencies ranges as opposed to the case for dry skin. This represents electromagnetic characteristics of biological tissues are altered by hydration conditions and wet tissues are more dispersive. FIG. 12B shows mechanical simulations results how strain affect center frequencies under two different hydration conditions, wet and dry, of biological tissues. These results indicate that large bandwidth of the stretchable antenna can accommodate mechanical deformation and hydration.

Thermal measurements: To mimic thermal characteristics of biological tissues, we put hydrogel film on top of a harvester. Then, we monitored variations of temperature on the surface between the LED and hydrogel by taking/calculating ultrathin thermal sensor which is implanted with depth of around 0.5 mm, as shown in FIG. 13. Then, we baked the system on the hot plate up to 37° C. During measurement, a harvester was powered wirelessly and operated with a stimulation condition (20 Hz, 10 ms duration for 10 mins) that is used in the behavior test. The temperature of the LED increased up to 44° C. and saturated at a given output power density of 70 mW/mm². At a given optical power density lower than 10 mW/mm², there are no noticeable variations of temperature during measurement.

TX configuration for stimulation of central nervous systems: FIG. 15A shows TX configuration for stimulation of central nerve systems with SAR distributions in the assay. Similarly, the TX systems comprises 4 antennas placed below the experimental assay, and this array system makes low profile beam patterns suitable for applications required for broad wireless coverage. SAR distributions on a mouse mesh model reveals that absorption of transmitted radio frequency energy into a body of a mouse is within the guideline suggested by IEEE or FCC. FIGS. 15B-15D explain a normalized transmission coefficient, S₁₂, when a TX system is on the XY plane and a mouse (or a harvester) is on the XY, YZ, and ZX plane, respectively. FIGS. 2C-2E show radiation patterns of the TX system and the harvester when the harvester (or the mouse) scans in the XY, YZ, ZX cut direction, respectively, and the results of simulations provide visual evidence of the robustness of operation in terms of position and orientation. When the harvester is located on the XY or YZ plane, its S₁₁ is comparable each other. However, the couplings are marginal when the harvester is located on the ZX plane. The modest coupling is mainly due to directional radiation patterns of the harvester when on the ZX plane as shown in FIG. 15E.

### REFERENCES FOR EXAMPLE 1

1. Zhang, F. et al. Optogenetic interrogation of neural circuits: technology for probing mammalian brain structures. Nature protocols 5, 439-456 (2010).

2. Boyden, E. S., Zhang, F., Bamberg, E., Nagel, G. & Deisseroth, K. Millisecond-timescale, genetically targeted optical control of neural activity. Nature neuroscience 8, 1263-1268 (2005).

3. Chow, B. Y. et al. High-performance genetically targetable optical neural silencing by light-driven proton pumps. Nature 463, 98-102 (2010).

4. Nagel, G. et al. Channelrhodopsin-2, a directly light-gated cation-selective membrane channel. Proceedings of the National Academy of Sciences of the United States of America 100, 13940-13945 (2003).

5. Haubensak, W. et al. Genetic dissection of an amygdala microcircuit that gates conditioned fear. Nature 468, 270-276 (2010).

6. Lee, J. H. et al. Global and local fMRI signals driven by neurons defined optogenetically by type and wiring. Nature 465, 788-792 (2010).

7. Hasegawa, H., Abbott, S., Han, B. X., Qi, Y. & Wang, F. Analyzing somatosensory axon projections with the sensory neuron-specific Advillin gene. The Journal of neuroscience: the official journal of the Society for Neuroscience 27, 14404-14414 (2007).

8. Gradinaru, V., Mogri, M., Thompson, K. R., Henderson, J. M. & Deisseroth, K. Optical deconstruction of parkinsonian neural circuitry. Science 324, 354-359 (2009).

9. Smith, K. M., Madden, J. F., Callister, R. J., Hughes, D. I. & Graham, B. A. The search for novel analgesics: re-examining spinal cord circuits with new tools. Frontiers in pharmacology 5, 22 (2014).

10. Iyer, S. M. et al. Virally mediated optogenetic excitation and inhibition of pain in freely moving nontransgenic mice. Nature biotechnology 32, 274-278 (2014).

11. Carr, F. B. & Zachariou, V. Nociception and pain: lessons from optogenetics. Frontiers in behavioral neuroscience 8, 69 (2014).

12. Daou, I. et al. Remote optogenetic activation and sensitization of pain pathways in freely moving mice. The Journal of neuroscience: the official journal of the Society for Neuroscience 33, 18631-18640 (2013).

13. Towne, C., Montgomery, K. L., lyer, S. M., Deisseroth, K. & Delp, S. L. Optogenetic control of targeted peripheral axons in freely moving animals. PloS one 8, e72691 (2013).

14. Gereau, R. W. t. et al. A pain research agenda for the 21st century. The journal of pain: official journal of the American Pain Society 15, 1203-1214 (2014).

15. Iyer, S. M. et al. Virally mediated optogenetic excitation and inhibition of pain in freely moving nontransgenic mice. Nature biotechnology (2014).

16. Daou, I. et al. Remote optogenetic activation and sensitization of pain pathways in freely moving mice. The Journal of neuroscience: the official journal of the Society for Neuroscience 33, 18631-18640 (2013).

17. Kozai, T. D. et al. Ultrasmall implantable composite microelectrodes with bioactive surfaces for chronic neural interfaces. Nature materials 11, 1065-1073 (2012).

18. Sparta, D. R. et al. Construction of implantable optical fibers for long-term optogenetic manipulation of neural circuits. Nature protocols 7, 12-23 (2012).

19. Jang, K. I. et al. Rugged and breathable forms of stretchable electronics with adherent composite substrates for transcutaneous monitoring. Nature communications 5, 4779 (2014).

20. Kim, D. H. et al. Epidermal electronics. Science 333, 838-843 (2011).

21. Kim, J., Kosaka, Y., Shimizu-Okabe, C., Niizaki, A. & Takayama, C. Characteristic development of the GABA-removal system in the mouse spinal cord. Neuroscience 262, 129-142 (2014).

22. Xu, S. et al. Soft microfluidic assemblies of sensors, circuits, and radios for the skin. Science 344, 70-74 (2014).

23. Kim, T.-i. et al. Injectable, cellular-scale optoelectronics with applications for wireless optogenetics. Science 340, 211-216 (2013).

24. Engineers, I.f.E.a.E., Vol. C95.1-2005 (2005).

25. Peyman, A., Rezazadeh, A. A. & Gabriel, C. Changes in the dielectric properties of rat tissue as a function of age at microwave frequencies. Physics in medicine and biology 46, 1617-1629 (2001).

26. Park, S. I. EHANCEMENT OF WIRELESS POWER TRANSMISSION INTO BIOLOGICAL TISSUES USING A HIGH SURFACE IMPEDANCE GROUND PLANE. Progress in Electromagnetics Research 135, 123-136 (2013).

27. Da Silva, S. et al. Proper formation of whisker barrelettes requires periphery-derived Smad4-dependent TGF-beta signaling. Proceedings of the National Academy of Sciences of the United States of America 108, 3395-3400 (2011).

28. Zhou, X. et al. Deletion of PIK3C3/Vps34 in sensory neurons causes rapid neurodegeneration by disrupting the endosomal but not the autophagic pathway. Proceedings of the National Academy of Sciences of the United States of America 107, 9424-9429 (2010).

29. Madisen, L. et al. A toolbox of Cre-dependent optogenetic transgenic mice for light-induced activation and silencing. Nature neuroscience 15, 793-802 (2012).

30. Mishra, S. K., Tisel, S. M., Orestes, P., Bhangoo, S. K. & Hoon, M. A. TRPV1-lineage neurons are required for thermal sensation. The EMBO journal 30, 582-593 (2011).

31. Agarwal, N., Offermanns, S. & Kuner, R. Conditional gene deletion in primary nociceptive neurons of trigeminal ganglia and dorsal root ganglia. Genesis (New York, N.Y.: 2000) 38, 122-129 (2004).

32. Fink, D. J. et al. Gene therapy for pain: results of a phase I clinical trial. Annals of neurology 70, 207-212 (2011).

33. Fink, D. J. & Wolfe, D. Gene Therapy for Pain: A Perspective. Pain management 1, 379-381 (2011).

34. Miyazato, M. et al. Suppression of detrusor-sphincter dyssynergia by herpes simplex virus vector mediated gene delivery of glutamic acid decarboxylase in spinal cord injured rats. J Urol 184, 1204-1210 (2010).

35. Yokoyama, H. et al. Gene therapy for bladder overactivity and nociception with herpes simplex virus vectors expressing preproenkephalin. Human gene therapy 20, 63-71 (2009).

36. Pleticha, J. et al. Preclinical toxicity evaluation of AAV for pain: evidence from human AAV studies and from the pharmacology of analgesic drugs. Mol Pain 10, 54 (2014).

37. Harrison, M. et al. Vertebral landmarks for the identification of spinal cord segments in the mouse. NeuroImage 68, 22-29 (2013).

38. Bennett, G. J. & Xie, Y. K. A peripheral mononeuropathy in rat that produces disorders of pain sensation like those seen in man. Pain 33, 87-107 (1988).

39. Golden, J. P. et al. Dopamine-dependent compensation maintains motor behavior in mice with developmental ablation of dopaminergic neurons. The Journal of neuroscience: the official journal of the Society for Neuroscience 33, 17095-17107 (2013).

40. Montana, M. C. et al. The metabotropic glutamate receptor subtype 5 antagonist fenobam is analgesic and has improved in vivo selectivity compared with the prototypical antagonist 2-methyl-6-(phenylethynyl)-pyridine. The Journal of pharmacology and experimental therapeutics 330, 834-843 (2009).

41. Golden, J. P. et al. RET signaling is required for survival and normal function of nonpeptidergic nociceptors. The Journal of neuroscience: the official journal of the Society for Neuroscience 30, 3983-3994 (2010).

### EXAMPLE 2

### Multichannel Wireless Operation

Optogenetic methods to modulate cells and signaling pathways via targeted expression and activation of light-sensitive proteins have greatly accelerated the process of mapping complex neural circuits and unraveling their biological purposes. Recently demonstrated technologies based on injectable, microscale inorganic light emitting diodes (μ-ILEDs) with wireless control and power delivery strategies offer important functionality in this context, by reducing the immune responses and eliminating the external tethers associated with traditional fiber optic approaches. Existing wireless μ-ILED embodiments allow, however, illumination only at a single targeted region of the brain with a single optical wavelength, and over spatial ranges of operation that are constrained by the radio frequency power transmission hardware. Provided herein are devices and schemes for multi-channel wireless operation of independently addressable, multi-color μ-ILEDs with fully implantable, miniaturized platforms. This advance, as demonstrated through in vitro and in vivo studies using thin, mechanically soft systems that separately control as many as three different μ-ILEDs, relies on specially designed stretchable antennas in which parallel capacitive coupling circuits yield several independent, well-separated operating frequencies, as verified through experiment and modeling results. When used in combination with active motion-tracking antenna arrays, these devices enable multi-channel optogenetic research on complex behavioral responses in groups of animals over large areas at low levels of radio frequency power (<1 W). Studies of the regions of the brain that are involved in sleep arousal and preference/aversion demonstrate the unique capabilities of these technologies.

Optogenetics exploits a toolbox of light-sensitive proteins for optical manipulation of neural networks as a powerful means for the study of circuit-level mechanisms that underlie psychiatric diseases [1-4]. Canonical optogenetic experiments in the brain require cranial insertion of an optical fiber to illuminate a region of interest [5, 6]. Although this approach permits simple behavior modeling, constraints in animal motion and alterations in natural behaviors due to fiber tethering and external fixation frustrate use in chronic longitudinal models and in experiments that assess complex responses. Many of these limitations can be bypassed with optoelectronics technologies and wireless receivers, as recently demonstrated in optogenetic stimulation of the brain, the peripheral nerves and the spinal cord [4, 7-13]. Systems that offer soft, compliant mechanical properties and thin, fully implantable designs are particularly advantageous [7]. The work reported here summarizes three key advances in this general class of technology; (1) designs for use in the deep brain, with examples in optogenetic control of sleep/wake transitions via stimulation of the locus coeruleus (LC) [14], (2) motion tracking systems for coordinated control over multiple transmission antennas, to allow ultralow power radio frequency operation over large areas and, most importantly, (3) specialized radio frequency antennas and associated circuits for independent control over multiple light sources in a single platform, with examples in optogenetic modulation of aversion/preference behaviors via dynorphinergic neurons in nucleus accumbens shell (NAcSh) using two-channel wireless devices for stimulation of separate regions of the brain individually, in a single mouse. This experimental demonstration is significant because, although activation of the dorsal and ventral regions of the NAcSh are known to generate preference and aversion behaviors, respectively, other approaches either cannot stimulate both regions in the same animal individually or simultaneously, or they cannot do so in a wireless, tether-free mode. The ability for independent, wireless power delivery and control over multiple light sources with emission wavelengths across the visible range also suggests related opportunities in optogenetic stimulation and inhibition via recently developed light sensitive proteins for advanced types of behavior experiments [15].

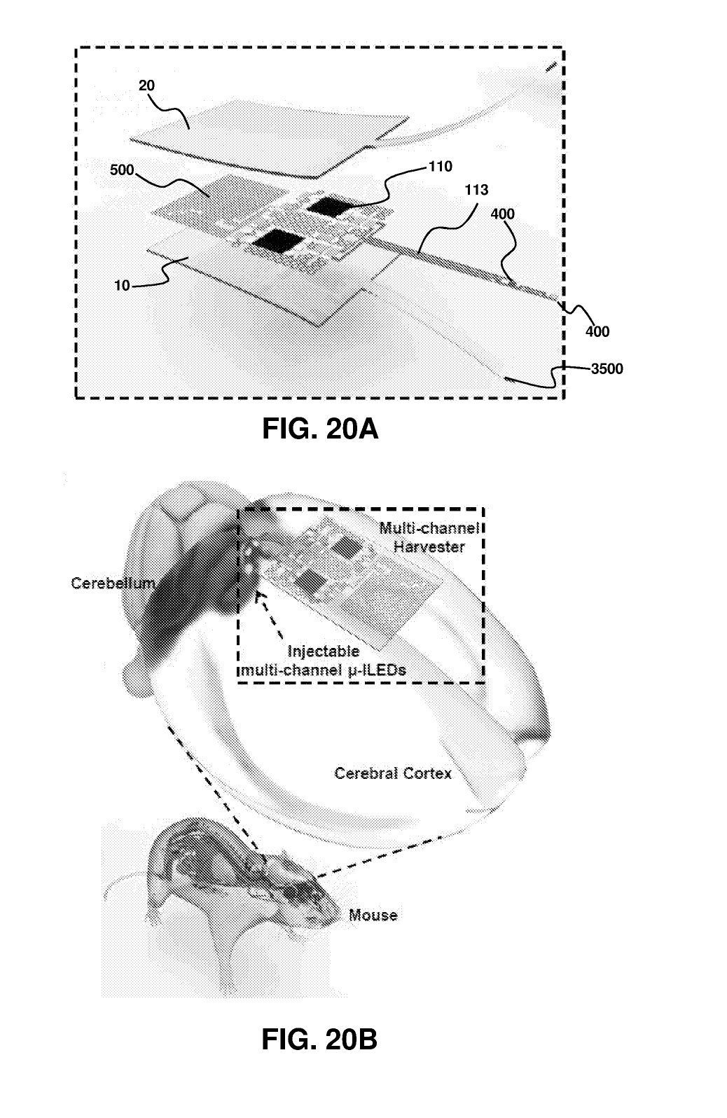

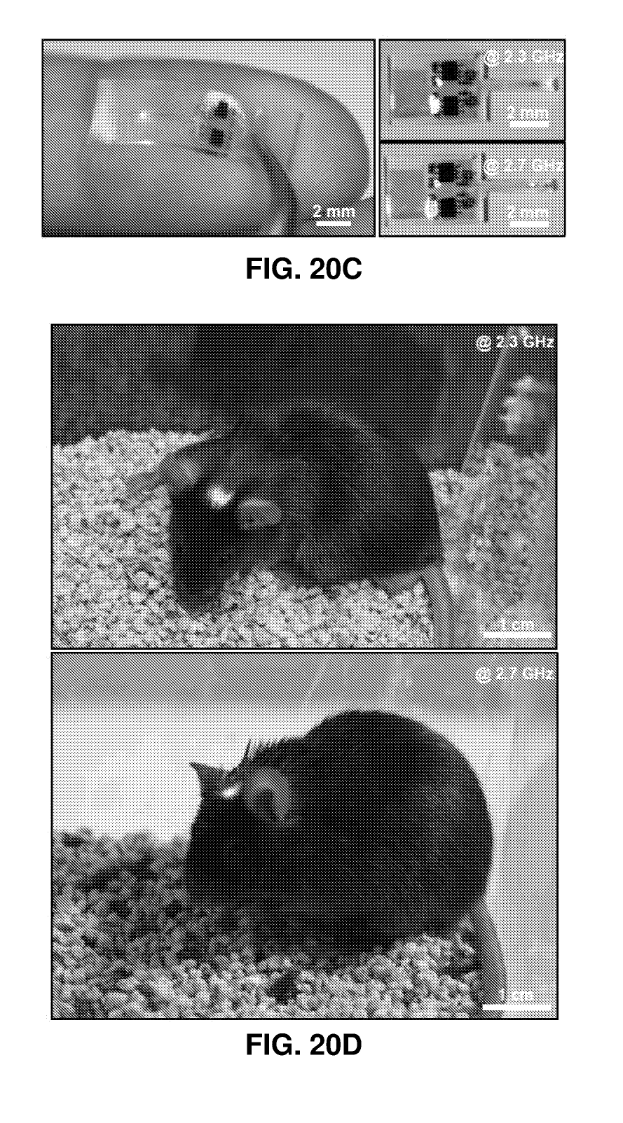

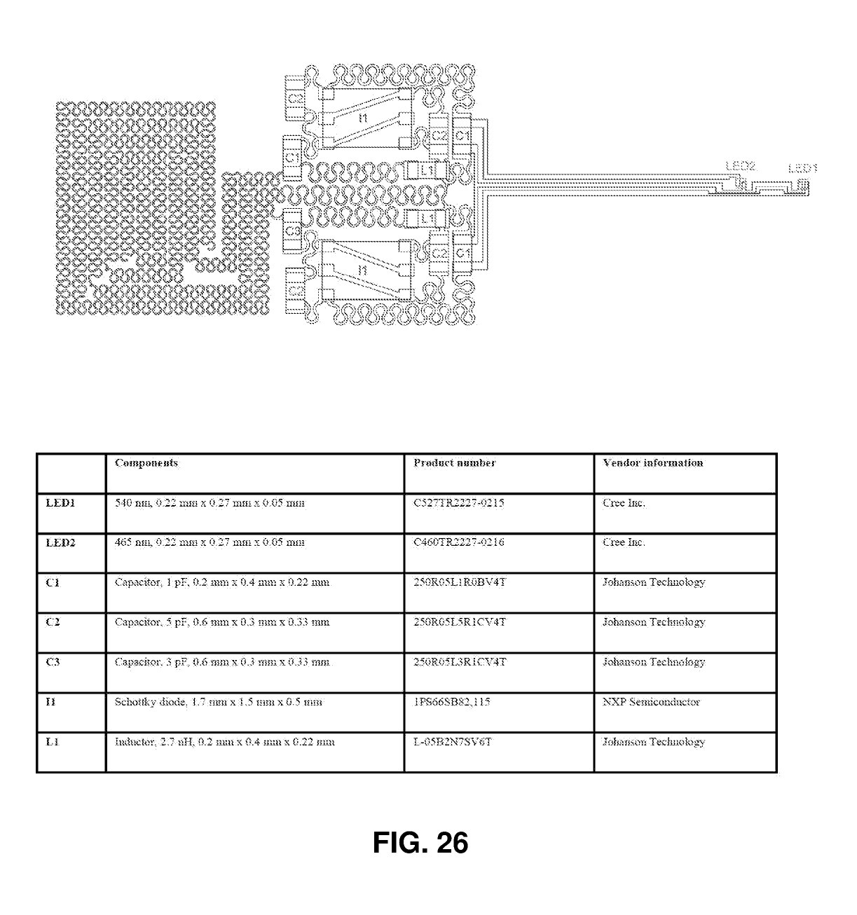

Optoelectronic design. The device platform enables separate, wireless operation of a collection of injectable microscale inorganic light emitting diodes (μ-ILEDs, based on unpackaged devices with dimensions of 220 μm width, 270 μm length, and 50 μm thickness). A radio frequency energy harvester receives signals from a transmitter, rectifies them, and triples the resulting voltage to provide a direct-current output for the μ-ILEDs. An impedance matching circuit, designed to maximize the received power, utilizes a ceramic chip capacitor (3 pF; 0.20 mm width, 0.4 mm length, 0.22 mm thickness) and an inductor (2.7 nH; 0.20 mm width, 0.4 mm length, 0.22 mm thickness) connected in series. The rectifier incorporates miniaturized Schottky diodes (1.7 mm width, 1.5 mm length, 0.5 mm thickness) and ceramic chip capacitors (5 pF; 0.20 mm width, 0.4 mm length, 0.22 mm thickness). The multiplier involves three Schottky diodes, identical to those in the rectifier, for the purpose of boosting the voltages provided by the rectifier (˜0.9 V) to values sufficient to operate the μ-ILEDs (˜2.7 V). To accommodate anatomical shapes and natural motions inside the targeted biological environment, the devices exploit principles of stretchable electronics in the form of serpentine Ti/Au interconnects passivated by layers of polyimide (40 μm thickness), with the entire system encapsulated in a low modulus silicone elastomer (˜0.5 MPa, 100 μm thickness for superstrate and substrate) to yield soft mechanics at the system level (effective modulus ˜1.7 MPa) (FIG. 20A) [7]. The fabrication procedures, along with details of the components and the circuit layouts are further described below and in FIG. 26.

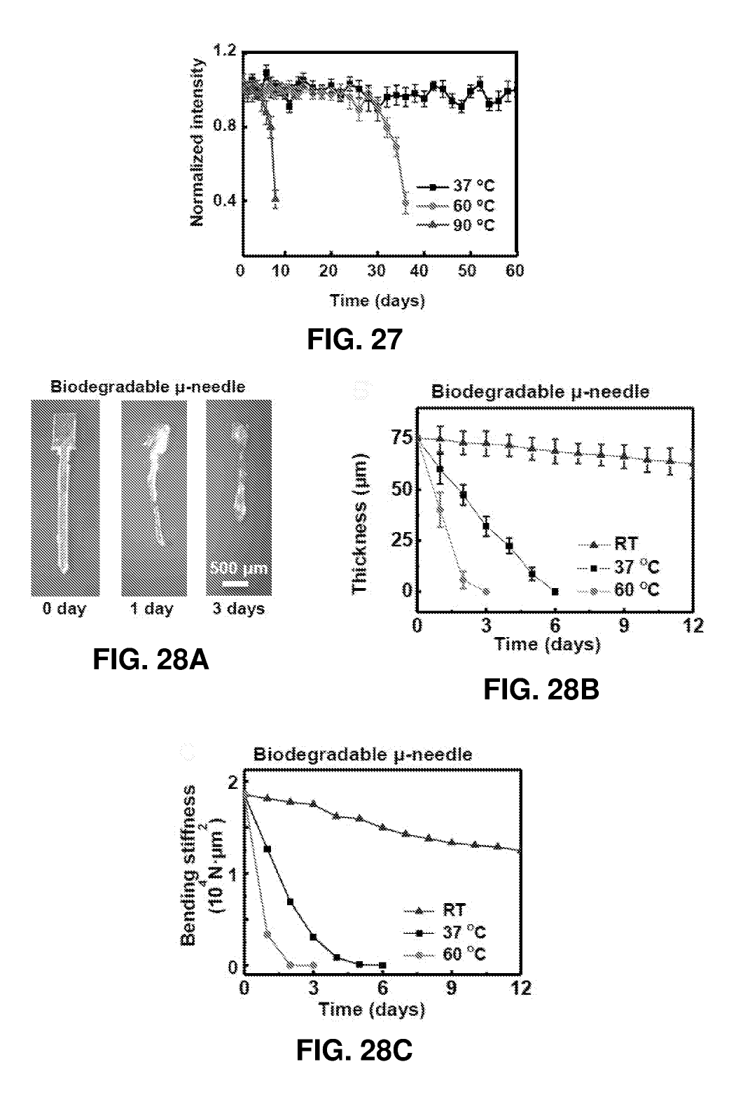

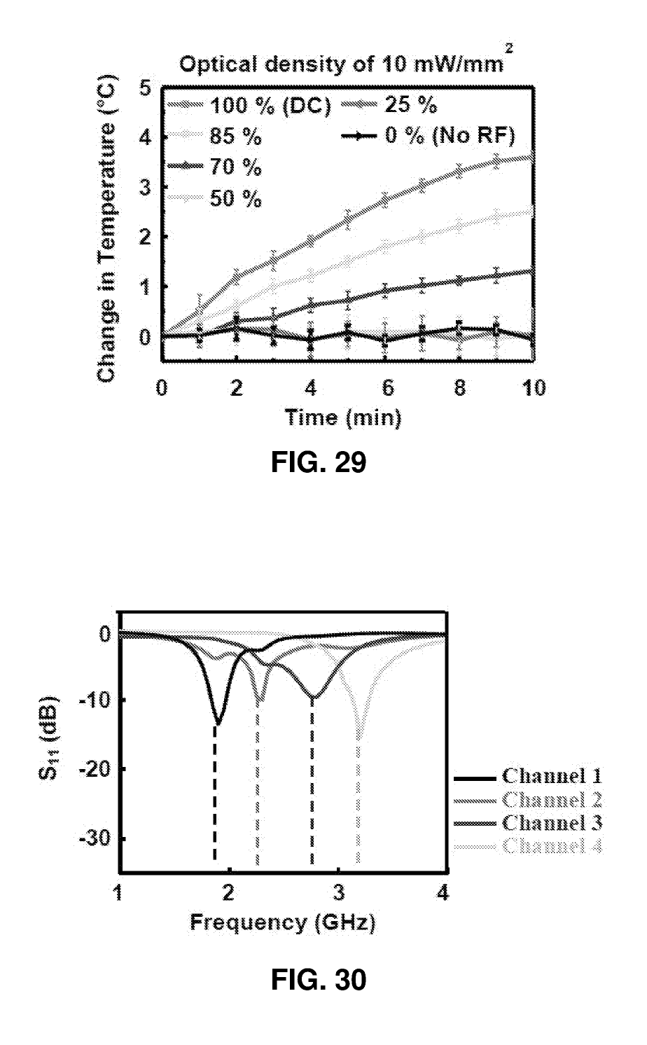

The thin, miniaturized geometry (4.3 mm width, and 8 mm length, 0.7 mm thickness), ultra-lightweight construction (33 mg), and mechanical compliance of these devices facilitate full implantation and chronic use in the brain (FIG. 20B). The system includes two parts: a back-end power harvesting and control unit, and an injectable needle-shaped substrate (μ-needle) that allows delivery of the μ-ILEDs (FIG. 20C; right top and bottom) to targeted structures in the deep brain. Both components offer soft mechanics to impose minimal constraints on natural motions of the surrounding tissue (FIG. 20C; left, FIG. 20D), with reliable operation under levels of deformation that significantly exceed those expected in freely moving animals [7]. Accelerated testing indicates that the soft silicone encapsulation preserves device functionality for 6 days when immersed in phosphate buffered saline (PBS, 7.4 pH) at 90° C., and the devices show no degradation after more than 2 months in similar solution at 37° C. (FIG. 27). Arrhenius-based extrapolations of testing at 60° C. and 90° C. suggest lifetimes of ˜6 months at 37° C. [16]. These soft, flexible components also reduce brain trauma upon insertion and minimize tissue damage after chronic implantation and activation [9, 10, 17]. The high degree of compliance of the injectable part of the system can, however, frustrate mechanical penetration into the brain tissue. Previous approaches exploit releasable injection microscale needles (μ-needles) to overcome this challenge [14]. Here, a biodegradable μ-needle made of poly lactic-co-glycolic acid (PLGA) obviates the need for extraction; this μ-needle provides sufficient stiffness for injection into regions of the deep brain such as the locus coeruleus (LC) and nucleus accumbens (NAc), but fully dissolves after exposure to biological fluids (FIG. 28A) [18-20]. In vitro testing shows that at 37° C. the μ-needles lose half of their thickness (an eight-fold reduction in their bending stiffness) in 3 days, with complete dissolution within a week (FIG. 28B-28C). Heat generation is also a potential concern for long-term implantation and operation of electrical devices in animals. Experimental studies using implantable thermal sensors show that optical power densities of 10 mW/mm² (50% duty cycle; 10 Hz period; 50 ms pulse width do not cause detectable temperature changes (FIG. 29).

Optical and electrical characteristics. One key feature of the devices described herein is that they support multi-channel operation, thereby overcoming a critical limitation of the single-channel, single-wavelength capabilities of previously reported wired and wireless systems [7-11, 13]. Here, an advanced, stretchable antenna structure integrates multiple capacitive coupling traces to yield non-overlapping resonances for frequency-selective harvesting and control. Each two-channel antenna consists of three serpentine lines (FIG. 21A; blue, red, and black line), where each pair (blue and red line, red and black line) involves resonant capacitive coupling with its neighbor. In particular, coupling between the blue and red traces (Channel 1, FIG. 21A) and between the red and black traces (Channel 2, FIG. 21A) enables operation at 2.3 (Channel 1) and 2.7 GHz (Channel 2), respectively. To illustrate this operation, computations of electric field magnitudes near the surfaces of the serpentine traces at a frequency of 2.3 GHz reveal enhanced capacitive coupling [21], and therefore resonant operation, associated with Channel 1 but not Channel 2, as featured in black and red dotted boxes (FIG. 21B; right). Such coupling can be captured by the reflection coefficient (S₁₁) for incident electromagnetic waves [7, 21]. Reductions in S₁₁ correspond to decreased reflections, and therefore improved power transmission efficiencies. The results exhibit two separate resonances, corresponding to two independently addressable channels of operation (FIG. 21C). The circuit diagram in FIG. 21D highlights the nature of signal flows from the antenna to the corresponding DC voltage output nodes. Images of an operating device demonstrate that the frequency of the incident RF radiation can be adjusted to activate the channels independently. The optical intensity generated in the non-targeted channel as a function of that in the targeted channel appears in FIG. 21E-21F. As indicated by the dotted lines, the intensity can reach ˜12 mW/mm², which is more than sufficient to activate optogenetic proteins, before the onset of the operation in the non-targeted channel [22]. Cross coupling between the two channels is negligible.

Advanced versions of the antenna design in FIG. 21A-21F can support independent manipulation of three separate channels. The scattering parameters of such an antenna that comprises 4 serpentine lines (FIG. 22A; red, black, blue, and green line) where each pair (red and black line, black and blue line, and blue and green line) exhibits capacitive coupling to its neighbor at a distinct frequency, indicate operation at 2.3 (Channel 1—black and red lines), 2.7 (Channel 2—black and blue lines), and 3.2 (Channel 3—blue and green lines) GHz. As before, computations of the electric field magnitude at 2.3 GHz illustrate selective capacitive coupling associated with the pair of traces that defines Channel 1. These finding are confirmed through a set of measurements and images of the devices (FIG. 22C-22G). As with the two-channel system, cross coupling can be ignored for the conditions of interest (FIG. 22D-22F). For both two and three-channel systems, the angular variations in power received at each channel can affect operation [4, 7, 21]. Calculations show that the radiation patterns associated with each channel, in all cases, are similar, ensuring that each receives (or transmits) an equivalent amount of power at a given orientation (FIG. 22H-22I). In addition, the serpentine layouts of the antennas and the soft, elastomeric substrate and superstrate afford linear elastic response to strain [7]. Tensile strains lead to shifting of the resonant frequencies to lower values, as expected from previously reported single channel antennas (FIG. 22J) [7]. This three-channel example highlights clearly the dramatic reduction in overall size compared to an otherwise equivalent arrangement of three separate, single-channel antennas (FIG. 22K). Advanced impedance matching techniques allow for further minimization of cross coupling, thereby extending of the number of channels that can be independently operated (FIG. 30) [21]. A practical limit for operation in the low gigahertz frequency ranges (0.7˜3.5 GHz, corresponding to a window of transparency for biological tissues) is ˜8 channels, based on a frequency range/channel margin of 2.8/0.4 [23, 24].

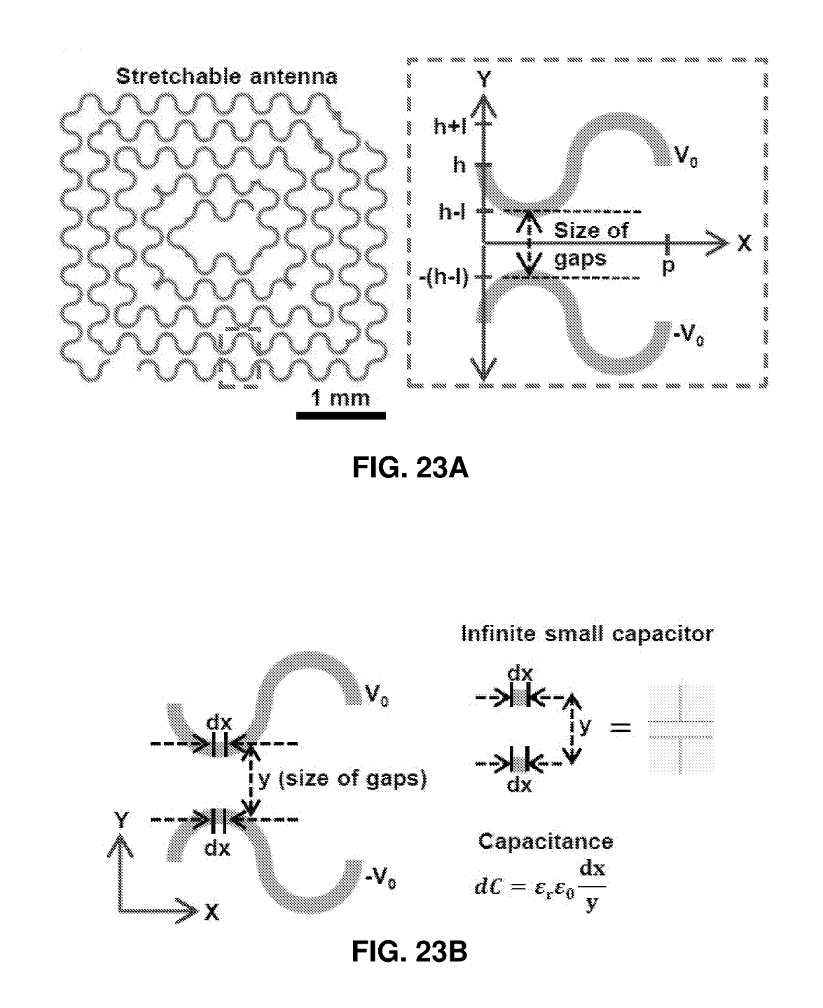

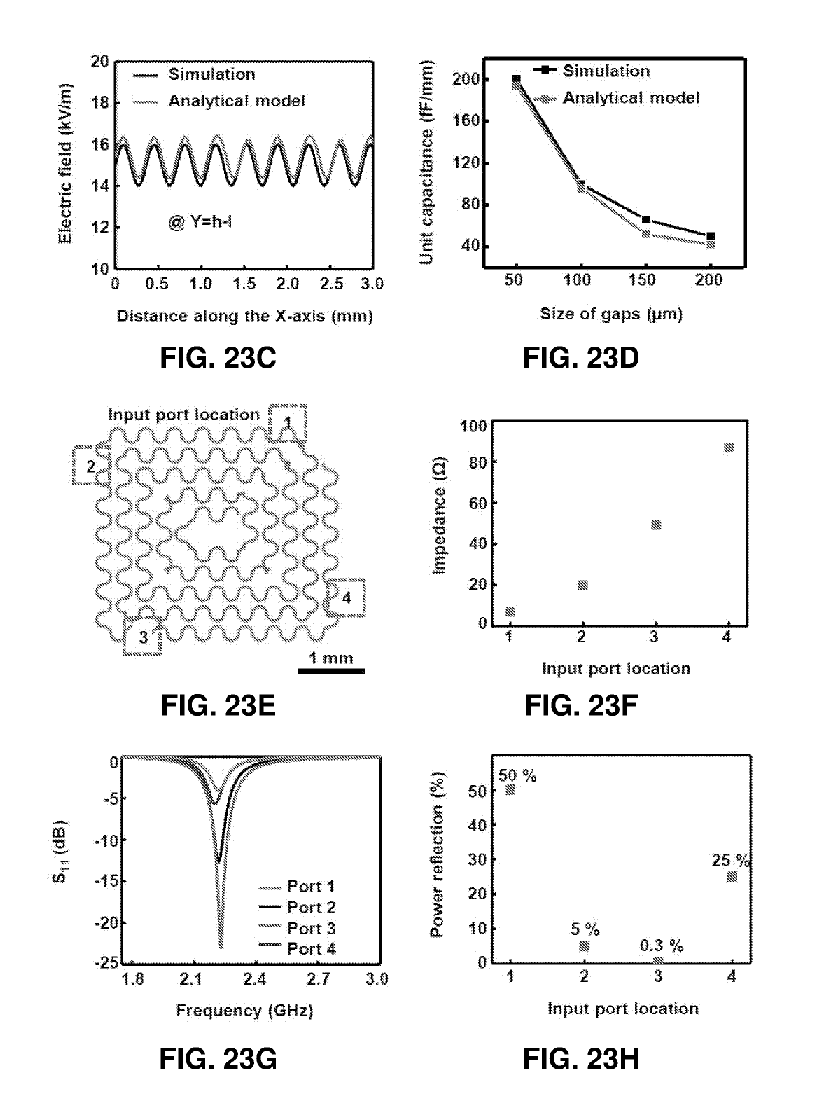

Modeling of the electromagnetic properties of stretchable antennas with serpentine designs. Multi-channel operation relies on the capacitive coupling that occurs between adjacent serpentine lines. This coupling can be captured analytically with solutions to the two-dimensional Laplace equation (FIG. 23A-23H) for simple antenna structures with serpentine geometries approximated by sinusoidal curves (FIG. 23A-23B). Here the electromagnetic interactions can be captured as a sum of infinitesimally small capacitors with gaps defined by the separation between adjacent lines (FIG. 23B). As the gap increases, the capacitive coupling decreases, with an analytical dependence that is consistent with numerical simulations (FIG. 23C-23D). These studies reveal that total capacitance of a stretchable antenna is approximately ˜1.5 pF. To achieve resonance in the low GHz ranges, inductances of 2˜3 nH are required, which is feasible within the dimensions of the antennas [21]. Similar strategies can be extended to calculations of the inductances to yield the resonance frequencies. This analytical approach dramatically reduces the computational burden of full simulations of the actual antenna layouts by providing a starting point for the designs. Moreover, the results confirm that the operation of these types of antennas relies critically on capacitive effects.

The input impedance of the antenna is also important, since proper impedance matching can maximize the power transmission efficiency. The designs exploited herein use an asymmetric geometry to increase the impedance over the very low values that are typically associated with antennas in this small size regime. Studies of input impedance as a function of the location of the input port provide insights into the role of this asymmetric geometry in input impedance and thus radiation efficiency. Moving the input from port 1 to port 4 breaks the symmetry, thereby increasing the impedance (FIG. 23E-23F) [21]. Using these concepts to realize impedances of 50 ohms ensures maximum power transfer [21]. The values of the reflection coefficient, S₁₁, are extremely sensitive to input impedance (FIG. 23G). The reflection values at each port, from 1 to 4, are 50, 5, 0.3, 25%, respectively (FIG. 23H).

Motion-tracking arrays of transmission antennas. Even with optimized impedance matching and antenna design, as the number of channels increases, corresponding increases in the total transmitted power may be necessary, depending on the nature of the optogenetic experiment. The upper limits may, with traditional systems, exceed exposure guidelines [23, 24]. An actively managed array of antennas and a separately controlled transmission system that tracks the motions of the animals, enables overall reductions in power by confining the delivering only to the relevant regions of an experimental assay. Here, digital image processing on data collected with a monitoring camera (FIG. 31A) locates the animal and then sends control signals to a multiplexer (MUX) that activates the antenna array in a way that directs power selectively to the corresponding area (FIG. 31B-31C) [25]. As such, transmission only occurs in the immediate environment of the targeted mouse, thereby greatly enhancing the efficiency of operation, to allow average power levels as low as 0.5 W (FIG. 32) for a typical case. This type of selective activation also lends itself well to complex behavioral assays such as conditioned place preference or those that demand coverage over large areas, social interactions, home cage manipulations or complex environments.

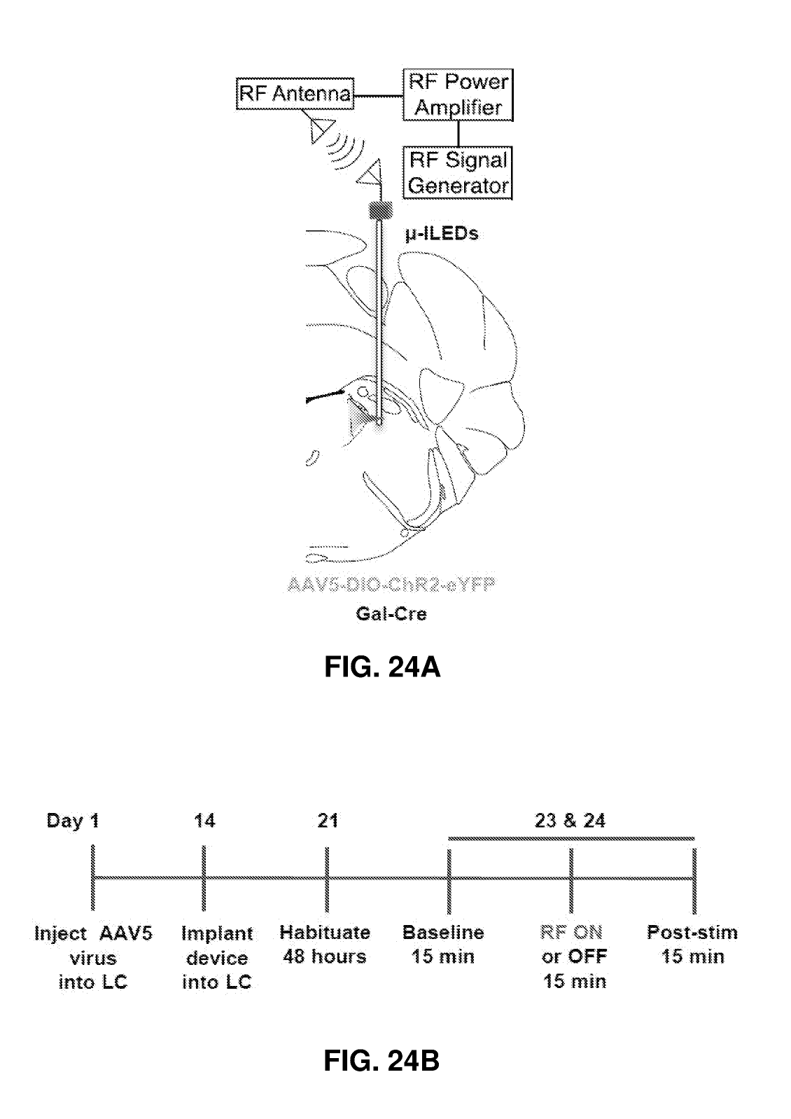

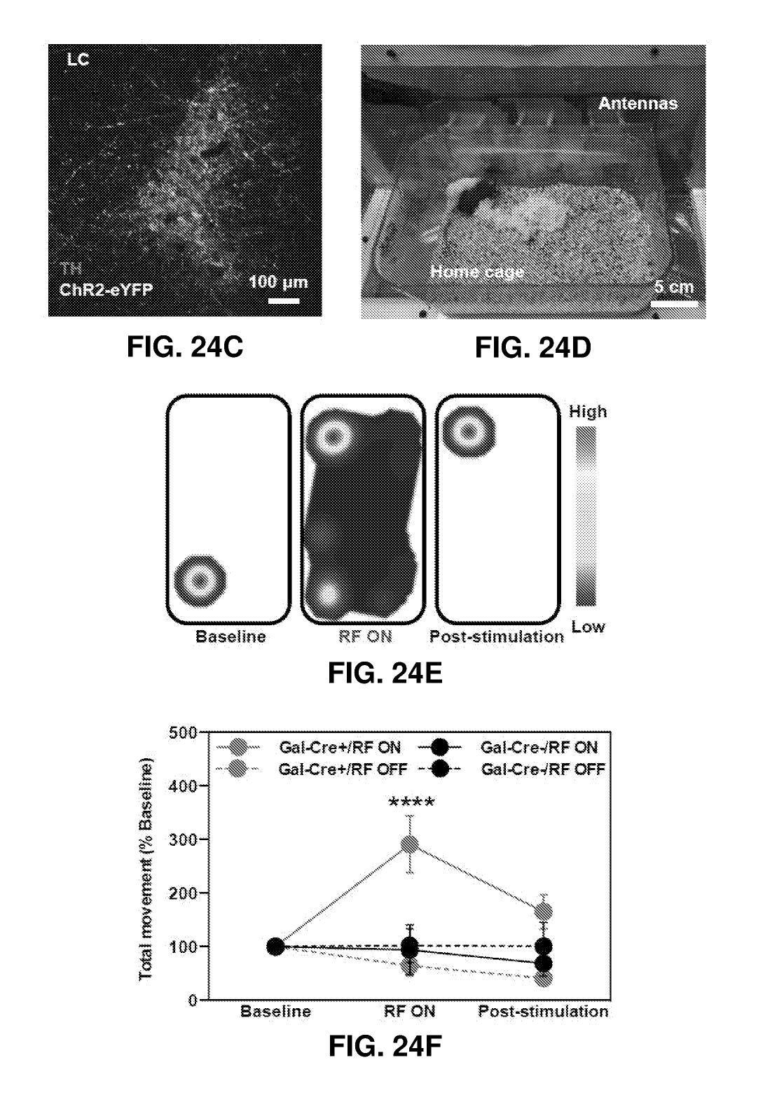

Optogenetic control of the locus coeruleus and nucleus accumbens shell. The unique features of the systems introduced herein demonstrate their utility for neural manipulation in native, homecage environments as well as standard behavioral assays with freely behaving mice. The LC has previously been implicated as a potent arousal center, such that activation of LC neurons can lead to sleep/wake transitions and speed emergence from anesthesia [26, 27]. We set out to show that these devices can optogenetically drive naturalistic behaviors within the home cage by wireless photostimulation of LC neurons. To do so, we used mice that express Cre recombinase under the promoter for galanin (Gal-Cre), which is expressed in the majority of noradrenergic LC neurons (LC-NE) in combination with virally transduced Cre-dependent expression of ChR2 [14, 26-28]. In these animals, the μ-ILED was placed within 1 mm of the LC to assess the ability of LC activation to drive locomotor activity during normal daily sleep periods (FIG. 24A-24B). Immunohistochemical analyses in Gal-Cre mice injected with AAV5-DIO-EF1α-ChR2 show robust ChR2 expression (yellow) in the LC that overlaps with tyrosine hydroxylase expression (red), consistent with expression selectively in the galaninergic subset of LC-NE neurons (FIG. 24C). We singly housed Cre-expressing (Cre+) and Cre-negative (Cre−) littermates in home cages mounted on custom built adapters that hold RF emitting antennas in close proximity to the home cage (˜5 cm from the cage wall and floor) (FIG. 31A, FIG. 24D). We followed a normal 12:12 hour light:dark cycle and recorded video footage of the mice during a period of normal sleep and recorded for three successive 15 minute periods. During the first and last periods, there was no activation of the implantable system (baseline and post-stimulation), but during the second period we stimulated the implanted device to drive LC activation by ChR2 under stimulation conditions (10 Hz, 50 ms pulse widths) (FIG. 24B) [14]. Photostimulation of Galanin-expressing LC neurons drove increased locomotor activity in Cre+ animals during the night phase compared to the pre-and post-activation period (FIG. 24E). In contrast, photostimulation of LC neurons in littermate Cre− mice did not alter the locomotor activity (FIG. 24F) (****p<0.0001 2-way Repeated measures ANOVA with Bonferroni post-hoc test). Taken together, these data demonstrate that activation of galanergic LC neurons produces arousal, and furthermore that these wireless devices can be easily used to perform behavioral experiments within the homecage, including in studies of sleep or resting that would otherwise be difficult with tethered cables.

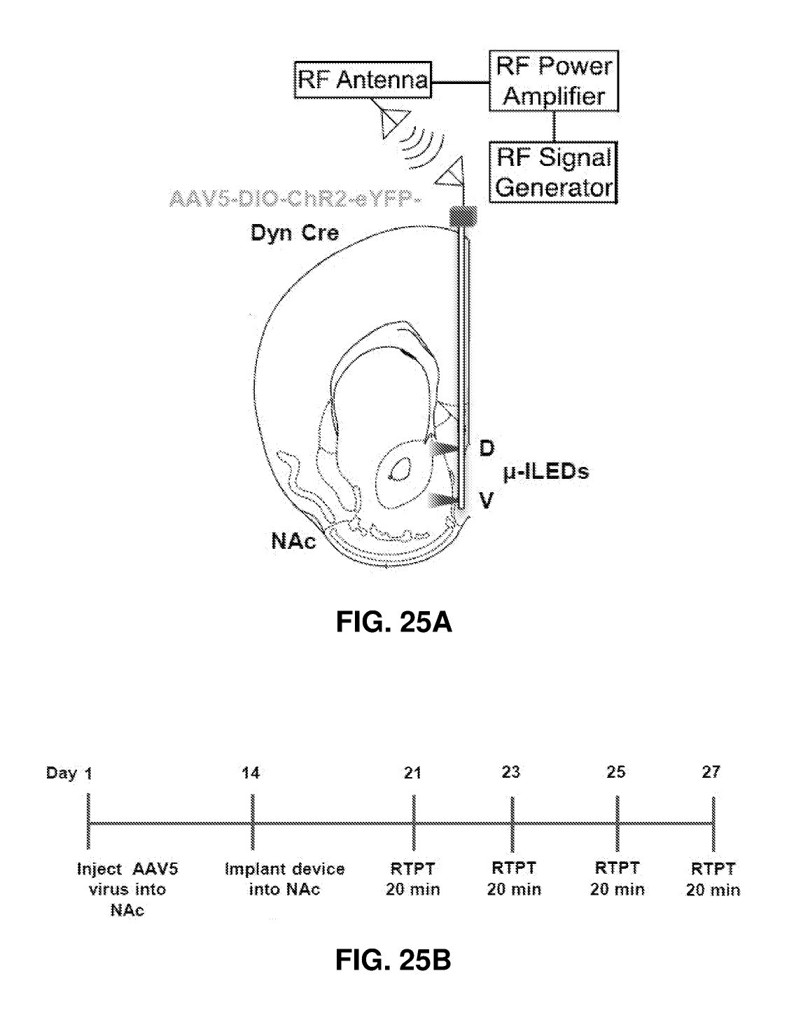

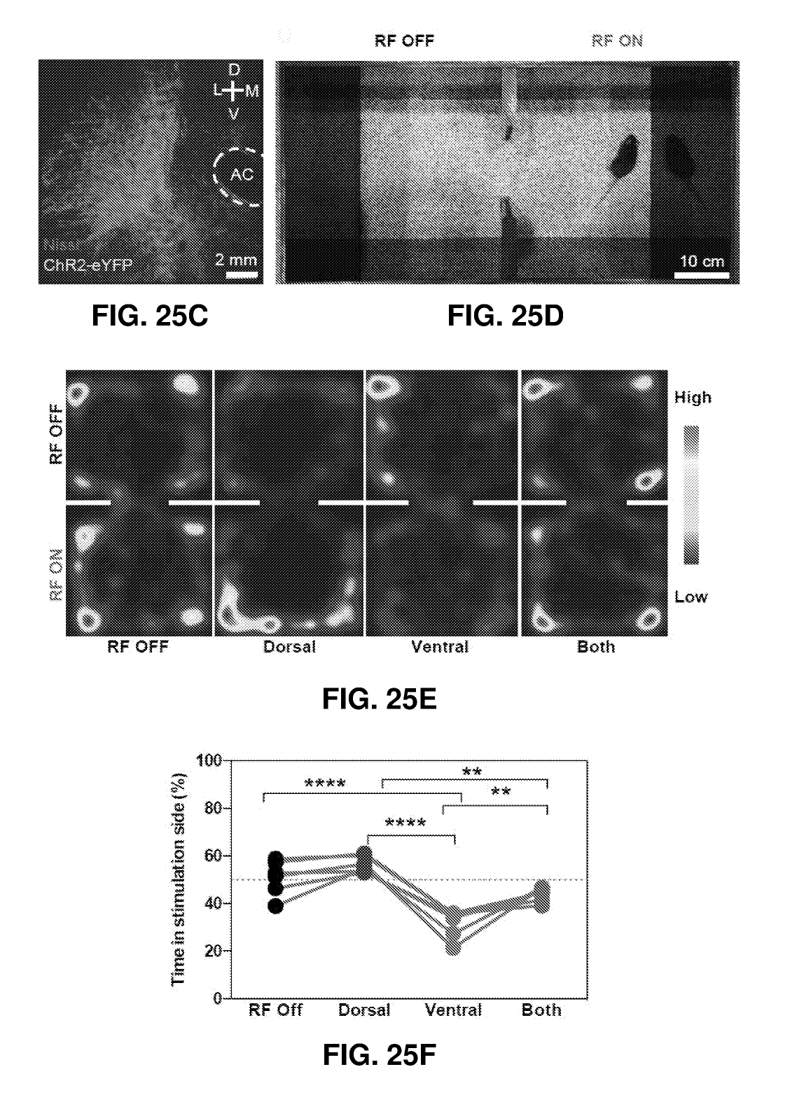

Multi-channel, bidirectional wireless control of reward and aversion. Wireless subdermal optogenetic probes that offer multichannel integration with discrete neuronal subpopulations are needed to extend our knowledge of complex neural circuitry. For example, multichannel capabilities are valuable in the context of experiments where multiple neuronal subpopulations need to be independently engaged. Recent efforts have shown that photostimulation of dynorphinergic neurons in the nucleus accumbens shell (NAcSh) is sufficient to induce both aversive and preference behaviors [12]. Photostimulation of dynorphinergic cells in the ventral NAcSh (vNAcSh) shell through activation of the kappa opioid receptor (KOR) elicits aversive behavior. Activation of nearby dorsal NAcSh (dNAcSh) dynorphin cells, however, in another KOR-mediated process elicits preference behavior. Characterization of this circuit previously required separate animals for optogenetic stimulation of the ventral and dorsal NAcSh regions. The multi-channel devices introduced here can define and modulate this spatially compact circuit with a single group of animals. Multi-channel devices implanted in preprodynorphin-IRES-cre positive mice that were infected with an AAV5-DIO-ChR2-eYFP virus to selectively express ChR2 in dynorphinergic neurons in the NAcSh demonstrate this capability (FIG. 25A-25C) [12]. Here, the vNAcSh targets one μ-ILED (Channel 1) and the nearby dNAcSh targets the other μ-ILED (Channel 2), separated by a distance of 1 mm (FIG. 25A). A custom-made unbiased, balanced two-compartment conditioning apparatus (52.5×25.5×25.5 cm, FIG. 25D) allows assessment of preference or aversion behavior by comparing the amount of time spent in the μ-ILED-on (RF-ON) side to the time spent in the μ-ILED-off side. Isolated photostimulation of the vNAcSh through activation of Channel 1 produces aversive behavior (FIG. 25E; ventral, FIG. 25F, and FIG. 33), while in the same animal illumination of the dNAcSh through activation of Channel 2 produces a real time preference behavior (FIG. 25E; dorsal, FIG. 25F, and FIG. 33; One-way ANOVA Bonferroni post-hoc; ****p<0.0001 RF off vs vNAcSh and dNAcSh vs vNAcSh and ***p<0.001 dNAcSh vs both and vNAcSh vs both). These data are consistent with prior studies, but here we show the subdermal multi-channel operation can photostimulate both regions in a single animal in a fully implantable tether free mode, thereby verifying that this circuit behaves as theorized [12]. These devices expand the range of complexity for in vivo optogenetic experiments that can be performed, and provide a flexible solution for multichannel functionality for neural circuit dissection.

The development of soft, fully implanted devices with independently controlled channels for multi-color illumination represents a significant technology advance for optogenetic studies. Simultaneous but separate optogenetic manipulation of multiple parts of the nervous system, as is demonstrated in this work, provides some examples of the possibilities. These multi-channel devices allow the direct interrogation of specific discrete circuits within the brain in individual animals. Studies to verify brain circuitry and function that have always been assumed or inferred through fMRI, anatomical, or pathophysiological studies can now be easily tested using these multi-channel platforms to stimulate, inhibit, or modulate the appropriate parts of the brain in relation to each other. Smart power delivery systems are essential, not only for the practical use of such multi-channel approaches in traditional experimental assays, but also to study long-range operation or multiplexed use across collections of animals, all with low-cost RF transmission hardware. While the immediate benefits of these advances on behavioral experiments are clear, they also enable the development of additional useful features such as wirelessly powered fully implantable RF controlled optofluidic systems and wireless closed loop optogenetic devices where one channel controls data transmission and the other channel offers power management; straightforward extensions of this platform will allow simultaneous sensing modalities and electrical stimulation where each function is assigned to each channel and controlled independently [29].

Methods: Fabrication of multi-channel devices. Fabrication was conducted on a clean glass slide (75 mm length, 50 mm width, and 1 mm thickness). Spin-casting polymethyl methacrylate (PMMA, 495 PMMA A8, Microchem) at 6000 rpm for 30 seconds and curing at 70° C. for 6 hours formed a coating with thickness of 200 nm. Subsequently, spin-casting a precursor to polydimethylsiloxane (PDMS, Sylgard 184) at 1000 rpm for 30 seconds and curing at 70° C. for 6 hours yielded an overcoat with thickness of 100 μm. An 18 μm thick copper foil (Dupont) laminated onto the PDMS, patterned by photolithography (AZ 5214E, AZ Electronic Materials) and copper etching defined the antenna and interconnects. Small amounts of lead-free solder paste (SMD290SNL250T5, Chipquik) applied to the surface mount devices and the μ-ILEDs (Cree Inc.) and baked at 285° C. in a vacuum oven for 10 minutes bonded the components to the patterned interconnects. An epoxy adhesive (200 nm), cured at 20° C. for 5 minutes, bonded this platform to the μ-needle. Spin-casting PDMS at 1000 rpm for 30 seconds and curing at 70° C. for 6 hours formed a final overcoat to complete the fabrication.

Fabrication of a biodegradable μ-needle. Hot pressing the poly (lactic-co-glycolic acid) (PLGA, 65:35, Sigma-Aldrich Inc., USA) thin film at 100° C. for 5 minutes, followed by laser-cutting, formed thin PLGA (˜75 μm thick) needle shaped substrates (μ-needles).

Electromagnetic simulations of multi-channel stretchable antennas. We used a commercially available finite element method tool, HFSS, to calculate the S11 values, the normalized electric field magnitudes, and angular radiation patterns with a mouse mesh model where layers corresponding to biological tissues are modeled as Cole-cole relaxation models [23].

Measurements of optical intensities. We used an optical measurement system (Extech LT300 Precision Digital Light Meter) to measure output of the LEDs. For accurate measurements of each individual channel, we prepared three types of samples, each with different color LED, to facilitate spectrally separate measurements of each channel.

Animals. Adult (25˜35 g) male mice were group-housed, given access to food pellets and water ad libitum, and maintained on a 12:12-hr light/dark cycle (lights on at 7:00 a.m). The Animal Care and Use Committee of Washington University approved all procedures which are conformed to NIH guidelines.

Stereotaxic surgery. For the arousal experiments, we used mice expressing Cre recombinase under the promoter for galanin (Gal-Cre) or littermate controls in combination with virally transduced Cre-dependent expression of ChR2 in order to target galaninergic neurons in the LC [30, 31]. For surgery, we anaesthetized the mice with isoflurane in an induction chamber and placed the animals in a stereotaxic frame that maintained anesthesia with 1%˜2% isoflurane through a nose cone. We injected AAVS-DIO-ChR2 unilaterally into the LC (coordinates from bregma: −5.45 anterior-posterior [AP], ±1.25 medial-lateral [ML], and −4.00 mm dorsal-ventral [DV]) in mice (Cre+ or Cre−) [28]. For multi-channel control of neuronal subpopulations experiments, we injected AAVS-DIO-ChR2-eYFP into the dorsal NAcSh of preprodynorphin-IRES-cre positive mice with targets of stereotaxic coordinates from bregma: +1.30 anterior-posterior [AP], ±0.5 medial-lateral [ML], −4.25 mm dorsal-ventral [DV] or ventral NAcSh stereotaxic coordinates from bregma: +1.30 [AP], ±0.5 [ML], −4.75 mm [DV] [12]. A minimum of two weeks after the viral injections, we stereotactically implanted the devices in mice using the same co-ordinates as above for AP and DV but at ±0.3 ML. Then, we secured the device to the skull with tissue glue and sutured the skin.

Behaviors. For the arousal experiments, we performed all behavioral experiments in a sound-attenuated room maintained at 23° C. at least 1 week following the final surgery. At 10:00 AM, during a period of normal sleep and less activity, we recorded the activity of the mice for three successive 15 minute periods by using Ethovision 8.5 (Noldus Information Technologies, RRID: rid_000100). During the first and last period, there was no activation of the implantable system. During the second period, the implanted system illuminated the LC under stimulation conditions (10 Hz, 50 ms pulse widths), which were previously shown to drive LC activation by ChR2 [14, 28]. For multi-channel control of neuronal subpopulations experiments, we placed mice in a custom-made unbiased, balanced two-compartment conditioning apparatus (52.5×25.5×25.5 cm) as described previously [12]. During a 20 min trial, entry into one compartment led to activation of the devices and provided photostimulation to the NAcSh (RF ON) (10 Hz, 50 ms pulse widths), while the animal remained in the light-paired chamber, and entry into the other chamber ended photostimulation (RF OFF). Arrangement of the transmitting antennas targeting only one side of the real-time place preference assay led to the unilateral RF transmission. With this configuration, the active side of the assay always received RF transmission (RF ON). The RF signal generator power output ranged from −10 to 0 dBm at 2.3 GHz, 2.5 GHz, and 2.7 GHz for each LED device.

Statistics/data analysis. We showed all grouped data as mean±s.e.m. Data were normally distributed, and independent Students' two-tailed, unpaired or paired t-tests determined differences between two groups. One-way or two-way analysis of variances (ANOVAs) followed by post-hoc Bonferroni or Dunnett's multiple comparisons if the main effect was significant at P<0.05 determined differences between multiple groups. We took statistical significance as *P<0.05, **P<0.01, ***P<0.001, ****P<0.0001 and conducted all analyses using Prism 5.0 (GraphPad).

Arrhenius model: To assess chronic reliability, we utilized extrapolations based on Arrhenius scaling of time to failure with temperature. By observing the behavior of devices at elevated temperatures, this scaling approach yields estimates for the time of failure at reduced temperatures. This form of accelerated testing utilizes the Arrhenius equation:

$$r = A exp ( \frac{- {E}_{a}}{kT} ) \qquad ( 1 )$$

where r=rate of the process, A=a proportional multiplier, Eₐ=the activation energy for the process, and k=Boltzmann constant.

Rearranging the equation yields a model for the temperature dependence of the failure time,

$$ln ( \frac{{t}_{2}}{{t}_{1}} ) = \frac{{E}_{a}}{k} ( \frac{1}{{T}_{2}} - \frac{1}{{T}_{1}} ) \qquad ( 2 )$$

where t₁=time to failure at T₁ and t₂=time to failure at T₂.

For T₁=90° C., T₂=60° C., experiments show that t₁=6 days and t₁=30 days. From these data, the value of Eₐ can be determined to be 60 kJ/(mol.K), similar to values in the literature [1]. This information allows extrapolation to time to failure at 37° C., which is 180 days (6 months).

Calculation of Electrostatic Potential and Capacitance

Assume that the length in the X-direction is much greater than the height in the Y-direction. The boundary function of the serpentine lines can be expressed by,

$$y = h - l cos ( kx ) where k = \frac{2 π}{p} \qquad ( 3 )$$

The electrostatic potential function, Ψ(x, y), satisfies the boundary and periodic conditions given by,

$$\begin{matrix}Ψ ( x , y ) = \{ \begin{matrix} {V}_{0} & if y = h - l cos ( kx ) \\ - {V}_{0} & if y = - h + l cos ( kx )\end{matrix} & ( 4 ) \\ Ψ ( x , y ) = Ψ ( x + p , y ) & ( 5 )\end{matrix}$$

Based on these conditions, one can solve Laplace equation,

$$\frac{{δ}^{2} Ψ ( x , y )}{{δx}^{2}} + \frac{{δ}^{2} Ψ ( x , y )}{{δy}^{2}} = 0 \qquad ( 6 )$$

beginning with a general solution that exploits separation of variables.

Ψ(x,y)=X(x)Y(y) (7)

Plugging the (7) into (6) and imposing the conditions in (4) and (5) leads to

$$\begin{matrix}{X}_{n} ( x ) = {a}_{n} cos ( nkx ) + {b}_{n} sin ( nkx ) n = 1 , 2 , 3 , … & ( 8 ) \\ {Y}_{n} ( y ) = {c}_{n} {e}^{nky} + {d}_{n} {e}^{- nky} n = 1 , 2 , 3 , … & ( 9 ) \\ a cos ( qx ) + b sin ( qx ) = a cos ( q ( x + p ) ) + b sin ( q ( x + p ) ) \\ where q , p = 2 π n , n = 1 , 2 , 3 , … & ( 10 ) \\ {Ψ}_{n} ( x , y ) = ( {a}_{n} cos ( nkx ) + {b}_{n} sin ( nkx ) ) [ {c}_{n} {e}^{nky} + {d}_{n} {e}^{- nky} ] , \\ n = 1 , 2 , 3 … & ( 11 ) \\ Ψ ( x , y ) = \frac{{v}_{0}}{h} y + \frac{{v}_{0}}{h} \frac{sin h ( ky ) cos ( kx )}{sin h ( k ( h - l cos ( kx ) ) )} k = \frac{2 π}{p} & ( 12 )\end{matrix}$$

The derivative of this expression with respect to y yields the Y components of the electric field,

$${E}_{Y} ( x , y ) = - \frac{{V}_{0}}{h} - \frac{{V}_{0}}{h} k \frac{cos h ( ky ) cos ( kx )}{sin h ( k ( h - l cos ( kx ) ) )} k = \frac{2 π}{p} \qquad ( 13 )$$

Next, we calculate the capacitance between two serpentine lines. This serpentine shaped-capacitor consists of infinite capacitors where capacitance is determined by (FIG. 23B),

$$dC = ɛ \frac{dx}{y} where y = 2 h + 2 l sin x \qquad ( 14 )$$

Integrating this expression along the length of the serpentine lines yields the capacitance.

$$C = \int dC = \int \frac{dx}{y} = \int \frac{dx}{2 h + 2 l sin x} \qquad ( 15 )$$

A motion tracking algorithm is developed using MATLAB code.

Thomas, T. H. & Kendrick, T. C. Thermal Analysis of Polydimethylsiloxanes. I. Thermal Degradation in Controlled Atmospheres. J. Polym. Sci. 7, 537-549 (1969).

### REFERENCES FOR EXAMPLE 2

1. Boyden, E. S., Zhang, F., Bamberg, E., Nagel, G. & Deisseroth, K. Millisecond-timescale, genetically targeted optical control of neural activity. Nat. Neurosci. 8, 1263-1268 (2005).

2. Adamantidis, A. R., Zhang, F., Aravanis, A. M. & Deisseroth, K. Neural substrates of awakening probed with optogenetic control of hypocretin neurons. Nature 450, 420-424 (2007).

3. Berndt, A., Lee, S., Ramakrishnan, C. & Deisseroth, K. Structure-guided transformation of a channelrhodopsin into a light-activated chloride channel. Science 344, 420-424 (2014).

4. Park, S.-I. et al. Ultraminiaturized photovoltaic and radio frequency powered optoelectronic systems for wireless optogenetics. J. Neural Eng. 12, 056002 (2015).

5. Deisseroth, K. Circuit dynamics of adaptive and maladaptive behavior. Nature 505, 309-317 (2014).

6. Paralikar, K. et al. An implantable optical stimulation delivery system for actuating an excitable biosubstrate. IEEE JSSC 46, 321-332 (2011).

7. Park, S.-I. et al. Soft, stretchable, fully implantable miniaturized optoelectronic systems for wireless optogenetics. Nat. Biotechnol. 33, 1280-1286 (2015).

8. Montgomery, K. L. et al. Wirelessly powered, fully internal optogenetics for brain, spinal and peripheral circuits in mice. Nat. Methods 12, 969-974 (2015).

9. Kim, T. et al. Injectable, cellular-scale optogenetics with applications for wireless optogenetics. Science 340, 211-216 (2013).

10. McCall, J. G. et al. Fabrication and application of flexible, multimodal light-emitting devices for wireless optogenetics. Nat. Protoc. 8, 2413-2428 (2013).

11. Siuda, E. R., et al. Optodynamic simulation of β-adrenergic receptor signaling. Nat. Commun. 6:8480 I DOI: 10.1038/ncomms9480 (2015).

12. Al-Hasani, R. et al. Distinct subpopulations of nucleus accumbens dynorphin neurons drive aversion and reward. Neuron 87, 1063-1077 (2015).

13. Iyer, S. M. et al. Virally mediated optogenetic excitation and inhibition of pain in freely moving nontransgenic mice. Nature Biotechnology 32, 274-278 (2014).

14. Carter, M. E. et al. Tuning arousal with optogenetic modulation of locus coeruleus neurons. Nat. Neurosci. 13, 1526-1533 (2010).

15. Berndt, A., Lee, S. Y., Ramakrishnan, C. & Deisseroth, K. Structure-guided transformation of channelrhodopsin into a light-activated chloride channel. Science 344, 420-424 (2014).

16. Kittel, C. & Kroemer, H. Thermal Physics 2nd edn W.H. Freeman (1980).

17. Kim, D.-H. et al. Epidermal electronics. Science 333, 838-843 (2011).

18. Hwang, S.-W. et al. A physically transient form of silicon electronics. Science 337, 1640-1644 (2012).

19. H. Tao, H. et al. Silk-based Resorbable Electronic Devices for Remotely Controlled Therapy and In Vivo Infection Abatement. Proc. Natl. Acad. Sci. USA 111(49), 17385-17389 (2014).

20. Hwang, S.-W. et al. 25th Anniversary Article: Materials for High-Performance Biodegradable Semiconductor Devices. Advanced Materials 26, 1992-2000 (2014).

21. Balanis, C. A. Antenna theory analysis and design 3rd edn Wiley-Interscience (2005).

22. Matteis, J. et al. Principles for applying optogenetics tools derived from direct comparative analysis of microbial opsins. Nat. Methods. 9, 159-172 (2012).

23. Park, S.-I. Enhancement of wireless power transmission into biological tissues using a high surface impedance ground plane. Prog. Electromagn. Res. 135, 123-136 (2013).

24. Gabriel, S., Lau, R. W. & Gabriel, C. Changes in the dielectric properties of rat tissue as a function of age at microwave frequencies. Phys. Med. Biol. 46, 1617-1629 (2001).

25. Adiv, G. Determining three-dimensional motion and structure from optical flow generated by several moving objects. IEEE Trans. Pattern Anal. Mach. Intell. 8, 381-401 (1985).

26. Vazey, E. M. & Aston-Jones, G. Designer receptor manipulations reveal a role of the locus coeruleus noradrenergic system in isoflurane general anesthesia. Proc. Natl. Acad. Sci. USA 111, 3859-3864 (2014).

27. Holets, V. R., Hokfelt, T., Rokaeus, A., Terenius, L. & Goldstein, M. Locus coeruleus neurons in the rat containing neuropeptide Y, tyrosine hydroxylase or galanin and their efferent projections to the spinal cord, cerebral cortex and hypothalamus. Neuroscience 24, 893-906 (1998).

28. McCall, J. G. et al. CRH Engagement of the Locus Coeruleus Noradrenergic System Mediates Stress-Induced Anxiety. Neuron 87, 605-620 (2015).

29. Jeong, J-W. et al. Wireless optofluidic systems for in vivo pharmacology and optogenetics. Cell 162, 662-674. (2015).

30. Gong, S. et al. A gene expression atlas of the central nervous system based on bacterial artificial chromosomes. Nature 425, 917-925 (2003).

31. Wu, Z., Autry, A. E., Bergan, J. F., Watebe-Uchida, M. & Dulac, C. G. Galanin neurons in the medial preoptic area govern parental behavior. Nature 509, 325-330 (2014).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components and method steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

The following references relate generally to fabrication methods, structures and systems for making electronic devices, and are hereby incorporated by reference to the extent not inconsistent with the disclosure in this application.

| Attorney | Application | | | Publication | | |
|---|---|---|---|---|---|---|
| Docket No. | No. | Filing Date | Publication No. | Date | Patent No. | Issue Date |
| 145-03 US | 11/001,689 | Dec. 1, 2004 | 2006/0286488 | Dec. 21, 2006 | 7,704,684 | Apr. 27, 2010 |
| 18-04 US | 11/115,954 | Apr. 27, 2005 | 2005/0238967 | Oct. 27, 2005 | 7,195,733 | Mar. 27, 2007 |
| 38-04A US | 11/145,574 | Jun. 2, 2005 | 2009/0294803 | Dec. 3, 2009 | 7,622,367 | Nov. 24, 2009 |
| 38-04B US | 11/145,542 | Jun. 2, 2005 | 2006/0038182 | Feb. 23, 2006 | 7,557,367 | Jul. 7, 2009 |
| 43-06 US | 11/421,654 | Jun. 1, 2006 | 2007/0032089 | Feb. 8, 2007 | 7,799,699 | Sep. 21, 2010 |
| 38-04C US | 11/423,287 | Jun. 9, 2006 | 2006/0286785 | Dec. 21, 2006 | 7,521,292 | Apr. 21, 2009 |
| 41-06 US | 11/423,192 | Jun. 9, 2006 | 2009/0199960 | Aug. 13, 2009 | 7,943,491 | May 17, 2011 |
| 25-06 US | 11/465,317 | Aug. 17, 2006 | — | — | — | — |
| 137-05 US | 11/675,659 | Feb. 16, 2007 | 2008/0055581 | Mar. 6, 2008 | — | — |
| 90-06 US | 11/782,799 | Jul. 25, 2007 | 2008/0212102 | Sep. 4, 2008 | 7,705,280 | Apr. 27, 2010 |
| 134-06 US | 11/851,182 | Sep. 6, 2007 | 2008/0157235 | Jul. 3, 2008 | 8,217,381 | Jul. 10, 2012 |
| 151-06 US | 11/585,788 | Sep. 20, 2007 | 2008/0108171 | May 8, 2008 | 7,932,123 | Apr. 26, 2011 |
| 216-06 US | 11/981,380 | Oct. 31, 2007 | 2010/0283069 | Nov. 11, 2010 | 7,972,875 | Jul. 5, 2011 |
| 116-07 US | 12/372,605 | Feb. 17, 2009 | — | — | — | — |
| 213-07 US | 12/398,811 | Mar. 5, 2009 | 2010/0002402 | Jan. 7, 2010 | 8,552,299 | Oct. 8, 2013 |
| 38-04D US | 12/405,475 | Mar. 17, 2009 | 2010/0059863 | Mar. 11, 2010 | 8,198,621 | Jun. 12, 2012 |
| 170-07 US | 12/418,071 | Apr. 3, 2009 | 2010/0052112 | Mar. 4, 2010 | 8,470,701 | Jun. 25, 2013 |
| 216-06A US | 12/522,582 | Jul. 9, 2009 | — | — | — | — |
| 38-04A1 US | 12/564,566 | Sep. 22, 2009 | 2010/0072577 | Mar. 25, 2010 | 7,982,296 | Jul. 19, 2011 |
| 71-07 US | 12/669,287 | Jan. 15, 2010 | 2011/0187798 | Aug. 4, 2011 | — | — |
| 60-09 US | 12/778,588 | May 12, 2010 | 2010/0317132 | Dec. 16, 2010 | — | — |
| 43-06A US | 12/844,492 | Jul. 27, 2010 | 2010/0289124 | Nov. 18, 2010 | 8,039,847 | Oct. 18, 2011 |
| 15-10 US | 12/892,001 | Sep. 28, 2010 | 2011/0230747 | Sep. 22, 2011 | 8,666,471 | Mar. 4, 2014 |
| 19-10 US | 12/916,934 | Nov. 1, 2010 | 2012/0105528 | May 3, 2012 | 8,562,095 | Oct. 22, 2013 |
| 3-10 US | 12/947,120 | Nov. 16, 2010 | 2011/0170225 | Jul. 14, 2011 | — | — |
| 118-08 US | 12/996,924 | Dec. 8, 2010 | 2011/0147715 | Jun. 23, 2011 | 8,946,683 | Feb. 3, 2015 |
| 126-09 US | 12/968,637 | Dec. 15, 2010 | 2012/0157804 | Jun. 21, 2012 | — | — |
| 50-10 US | 13/046,191 | Mar. 11, 2011 | 2012/0165759 | Jun. 28, 2012 | — | — |
| 151-06A US | 13/071,027 | Mar. 24, 2011 | 2011/0171813 | Jul. 14, 2011 | — | — |
| 137-05A US | 13/095,502 | Apr. 27, 2011 | — | — | — | — |
| 216-06B US | 13/100,774 | May 4, 2011 | 2011/0266561 | Nov. 3, 2011 | 8,722,458 | May 13, 2014 |
| 38-04A2 US | 13/113,504 | May 23, 2011 | 2011/0220890 | Sep. 15, 2011 | 8,440,546 | May 14, 2013 |
| 136-08 US | 13/120,486 | Aug. 4, 2011 | 2011/0277813 | Nov. 17, 2011 | 8,679,888 | Mar. 25, 2014 |
| 151-06B US | 13/228,041 | Sep. 8, 2011 | 2011/0316120 | Dec. 29, 2011 | — | — |
| 43-06B US | 13/270,954 | Oct. 11, 2011 | 2012/0083099 | Apr. 5, 2012 | 8,394,706 | Mar. 12, 2013 |
| 3-11 US | 13/349,336 | Jan. 12, 2012 | 2012/0261551 | Oct. 18, 2012 | — | — |
| 38-04E US | 13/441,618 | Apr. 6, 2012 | 2013/0100618 | Apr. 25, 2013 | 8,754,396 | Jun. 17, 2014 |
| 134-06B US | 13/441,598 | Apr. 6, 2012 | 2012/0327608 | Dec. 27, 2012 | 8,729,524 | May 20, 2014 |
| 28-11 US | 13/472,165 | May 15, 2012 | 2012/0320581 | Dec. 20, 2012 | — | — |
| 7-11 US | 13/486,726 | Jun. 1, 2012 | 2013/0072775 | Mar. 21, 2013 | 8,934,965 | Jan. 13, 2015 |
| 29-11 US | 13/492,636 | Jun. 8, 2012 | 2013/0041235 | Feb. 14, 2013 | — | — |
| 84-11 US | 13/549,291 | Jul. 13, 2012 | 2013/0036928 | Feb. 14, 2013 | — | — |
| 25-06A US | 13/596,343 | Aug. 28, 2012 | 2012/0321785 | Dec. 20, 2012 | 8,367,035 | Feb. 5, 2013 |
| 150-11 US | 13/624,096 | Sep. 21, 2012 | 2013/0140649 | Jun. 6, 2013 | — | — |
| 38-04A3 US | 13/801,868 | Mar. 13, 2013 | 2013/0320503 | Dec. 5, 2013 | 8,664,699 | Mar. 4, 2014 |
| 125-12 US | 13/835,284 | Mar. 15, 2013 | 2014/0220422 | Aug. 7, 2014 | — | — |
| 30-13 US | 13/853,770 | Mar. 29, 2013 | 2013/0333094 | Dec. 19, 2013 | — | — |
| 213-07A US | 13/974,963 | Aug. 23, 2013 | 2014/0140020 | May 22, 2014 | 8,905,772 | Dec. 9, 2014 |
| 19-10A US | 14/033,765 | Sep. 23, 2013 | 2014/0092158 | Apr. 3, 2014 | — | — |
| 15-10A US | 14/140,299 | Dec. 24, 2013 | 2014/0163390 | Jun. 12, 2014 | — | — |
| 38-04A4 US | 14/155,010 | Jan. 14, 2014 | 2014/0191236 | Jul. 10, 2014 | — | — |
| 136-08A US | 14/173,525 | Feb. 5, 2014 | 2014/0216524 | Aug. 7, 2014 | — | — |
| 216-06C US | 14/209,481 | Mar. 13, 2014 | 2014/0373898 | Dec. 25, 2014 | — | — |
| 134-06C US | 14/220,910 | Mar. 20, 2014 | 2014/0374872 | Dec. 25, 2014 | — | — |
| 38-04F US | 14/220,923 | Mar. 20, 2014 | 2015/0001462 | Jan. 1, 2015 | — | — |
| 151-06C US | 14/246,962 | Apr. 7, 2014 | 2014/0361409 | Dec. 11, 2014 | — | — |
| 62-13 US | 14/250,671 | Apr. 11, 2014 | 2014/0305900 | Oct. 16, 2014 | — | — |
| 56-13 US | 14/251,259 | Apr. 11, 2014 | 2014/0323968 | Oct. 30, 2014 | — | — |
| 60-09A US | 12/778,588 | Sep. 5, 2014 | 2015/0132873 | May 14, 2015 | — | — |
| 84-13 US | 14/504,736 | Oct. 2, 2014 | 2015/0141767 | May 21, 2015 | — | — |
| 213-07B US | 14/521,319 | Oct. 22, 2014 | — | — | — | — |
| 7-11A US | 14/532,687 | Nov. 4, 2014 | 2015/0080695 | Mar. 19, 2015 | — | — |
| 2-14 US | 14/599,290 | Jan. 16, 2015 | — | — | — | — |
| 71-07A US | 12/669,287 | Apr. 14, 2015 | — | — | — | — |
| 213-07C US | 12/398,811 | May 7, 2015 | — | — | — | — |
| 15-13 WO | PCT/US2014/ | Feb. 19, 2014 | WO2014/126927 | Aug. 21, 2014 | — | — |
| | 015825 | | | | | |
| 128-13 WO | PCT/US2014/ | Feb. 5, 2014 | WO 2014/124044 | Aug. 14, 2014 | — | — |
| | 014932 | | | | | |
| 8-14 WO | PCT/US2014/ | Feb. 18, 2014 | WO 2014/124049 | Aug. 14, 2014 | — | — |
| | 014944 | | | | | |
| 35-13 WO | PCT/US2014/ | Mar. 6, 2014 | WO 2014/138465 | Sep. 12, 2014 | — | — |
| | 021371 | | | | | |
| 54-13 WO | PCT/US2014/ | Apr. 3, 2014 | WO 2014/165686 | Oct. 9, 2014 | — | — |
| | 032848 | | | | | |

Every formulation or combination of components described or exemplified herein can be used to practice the invention, unless otherwise stated.

Whenever a range is given in the specification, for example, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified. As used herein, “consisting essentially of” does not exclude materials or steps that do not materially affect the basic and novel characteristics of the claim. In each instance herein, any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described.

We claim:

1. A fully implantable soft medical device comprising:
an elastomeric substrate;
a stretchable electronic device supported by said substrate, wherein said stretchable electronic device comprises:
an electronic component configured to interface with biological tissue;
a wireless power and control system for wirelessly powering and receiving a control signal for controlling said electronic components; and
an elastomeric superstrate that covers at least a portion of a top surface of said stretchable electronic device.

2. The device of claim 1, wherein said stretchable and wireless power and control system comprises a NFC chip device.

3. The device of claim 1, wherein said wireless power and control system comprises a stretchable radio frequency antenna having:
a plurality of adjacent serpentine electrical conductors separated by a separation distance, wherein adjacent serpentine electrical conductors are capacitatively coupled to each other.

4. The device of claim 3, wherein said plurality of adjacent serpentine electrical conductors provide a bandwidth of between 200 MHz and 300 MHz with a center frequency of between 2 GHz and 2.5 GHz over a strain range of up to 25% in a horizontal, a vertical or a horizontal and vertical direction.

5. The device of claim 1, wherein said electronic device has a thickness less than or equal to 100 μm and said medical device has a total thickness less than 1 mm.

6. The device of claim 1, wherein said power system comprises:
a magnetic loop antenna and an externally located electrode for generating an electric field over said magnetic loop antenna to power said electronic device.

7. The device of claim 1, wherein said control system comprises:
an externally located transmitter configured to transmit said control signal to said electronic device; and
a radio frequency harvester operably connected to the electronic device for receiving said control signal and subsequent control of said electronic components.

8. The device of claim 7, wherein said control system further comprises:
an impedance matching circuit;
a voltage multiplier; and
wherein a received power from said power system is converted into a direct current output by said impedance matching circuit and voltage multiplier for said control of said electronic components.

9. The device of claim 7, wherein said radio frequency harvester comprises a stretchable radio frequency antenna.

10. The device of claim 9, wherein said stretchable radio frequency antenna comprises adjacent serpentine electrical conductors in capacitative connection with each other.

11. The device of claim 1, wherein said stretchable electronic device is configured for one or more of:
electrical stimulation, electrical monitoring, or both;
optical stimulation, optical monitoring, or both;
controlled delivery of a biotherapeutic agent;
thermal control or sensing; or
pressure sensing;

12. The device of claim 1, configured for interfacing with a nerve or neural tissue.

13. The device of claim 9, configured for interfacing with a peripheral nerve.

14. The device of any of claims 12-13, wherein said electronic components comprise a light source to provide a rapid and temporally controllable optical stimulation.

15. The device of claim 14, having an average optical output power density of between 9.5 mW/mm² and 10 mW/mm² over a target region during device activation and configured for use in an optogenetic application.

16. The device of claim 14, wherein said light source comprises one or more μLEDs.

17. The device of claim 1, configured for slideable insertion into a muscle pocket of a living animal.

18. The device of claim 1, further comprising a pair of bilateral wings connected to or extending from the device and configured for suturing to a surrounding tissue for stable positioning of the device after implantation.

19. The device of claim 1, configured for chronic wireless implantation and remote control for up to one year.

20. The device of claim 1, wherein the device has a bulk Young's modulus that is matched to soft tissue and is less than or equal to 5 MPa and a bending stiffness of per unit width that is less than or equal to 10⁻⁷N m.

21. The device of claim 1, having a device footprint area that is less than or equal to 500 mm².

22. The device of claim 21, wherein said device footprint area is greater than or equal to 1 mm².

23. The device of claim 1, wherein said substrate has an average Young's modulus that is less than or equal to 10 MPa.

24. The device of claim 23, wherein said average Young's modulus is greater than 0.5 kPa.

25. The device of claim 1, wherein said electronic components comprise a plurality of independently addressable electronic components for a plurality of independently addressable interfacing with biological tissue.

26. The device of claim 25, wherein said electronic components comprise at least one actuator and at least one sensor to provide simultaneous and independent control of tissue activation with said actuator and tissue sensing with said sensor.

27. The device of claim 25, wherein said electronic components comprise a plurality of independently addressable LED optical sources.

28. The device of claim 27, wherein said independently addressable LED optical sources are each independently characterized by an emitting area less than or equal to 1×10⁵ μm².

29. The device of claim 28, wherein said independently addressable LED optical sources are each independently characterized by an emitting area selected from the range of 1×10³ μm² to 1×10⁵ μm².

30. The device of claim 27, wherein said independently addressable LED optical sources are provided in a 1D or 2D array.

31. The device of claim 27, wherein said independently addressable LED optical sources are operationally connected to a plurality of stretchable antenna structures providing for independent control of said LED optical sources.

32. The device of claim 31, wherein said stretchable antenna structures integrate multiple capacitive coupling traces to provide non-overlapping resonance frequencies for selective energy harvesting and control of an input radiofrequency.

33. The device of claim 27, wherein at least a portion of said independently addressable LED optical sources provide light characterized by a different emission wavelength spectrum.

34. The device of claim 25, wherein said plurality of independently addressable electronic components comprises up to eight independently addressable electronic components for an up to eight-channel multiplexing.

35. The device of claim 1, further comprising a motion tracking system in operational connection with said stretchable electronic circuit for tracking a motion of the devices when in use.

36. The device of claim 35, wherein said motion tracking system provides for a confined power delivery to said device over a power area.

37. The device of claim 1, further comprising a biodegradable needle operationally connected to said electronic circuit, said biodegradable needle having a stiffness sufficient to allow for injection or implantation of the device in a biological tissue.

38. The device of claim 1, wherein said wireless power and control system is stretchable and capable of accommodating a strain that is greater than 10% without fracture.

39. A wireless method of interfacing with biological tissue, the method comprising the steps of:
providing in a patient a soft medical device comprising:
an elastomeric substrate;
a stretchable electronic device supported by said substrate, wherein said stretchable electronic device comprises:
one or more electronic components configured to interface with biological tissue;
a wireless power and control system for wirelessly powering and receiving a control signal for controlling said electronic components;
an elastomeric superstrate that covers at least a portion of a top surface of said stretchable electronic device; and
generating a control signal with an externally located control signal generator to wirelessly control said electronic device, thereby interfacing with biological tissue adjacent to the soft medical device.

40. The method of claim 39, further comprising the step of powering said medical device by an externally-generated radiofrequency signal.

41. The method of claim 39, wherein said externally generated control signal is received and processed by said medical device with a NFC device that is operably connected to said electronic device.

42. The method of any of claims 39-41, wherein the soft medical device interfaces with a peripheral nerve tissue.

43. The method of any of claims 39-41, wherein the device is epidurally implanted for a pain relief application.

44. The method of any of claims 39-41, wherein the interfacing with biological tissue is by one or more of: optically interfacing; electrically interfacing, thermally interfacing;
chemically interfacing; or pressure interfacing.

45. The method of claim 44 wherein the interfacing comprises optical stimulation of a tissue adjacent to the soft medical device, wherein at least a portion of the cells in said tissue have been genetically transformed to express light-sensitive proteins having a light-intensity dependent functional activity.

46. The method of any of claims 39-41, wherein said soft medical device is configured to conform to a desired tissue or to a desired shape upon implantation without substantial impact on device functionality.

47. The method of any of claims 39-41, wherein the device is capable of a flexibility to accommodate a radius of curvature that is as small as 50 μm.

48. The method of any of claim 39-41, wherein the electronic components comprise a plurality of independently addressable electronic components, the method further comprising the step of independently wirelessly powering and controlling said plurality of independently addressable electronic components.

49. The method of any of claims 39-41, wherein the electronic components comprise a plurality of independently addressable LED optical sources, the method further comprising the step of independently wirelessly powering at least one of said plurality of independently addressable LED optical sources to provide optical stimulation.

50. The method of claim 39, wherein soft medical implants are implanted in additional patients, the method further comprising the step of: providing a multiplex control for the plurality of medical devices in the plurality of patients.

51. A multi-channel fully implantable medical device comprising:
a substrate;
an electronic device supported by said substrate, wherein said electronic device comprises:
a plurality of independently addressable electronic components configured to interface with biological tissue;
a multi-channel antenna in electronic contact with said plurality of independently addressable electronic components for controlling said electronic components;
a superstrate that at least partially covers said electronic device.

52. The device of claim 51, wherein said electronic components comprise a plurality of actuators, a plurality of sensors, or at least one actuator and at least one sensor, wherein each of the electronic components are independently addressable.

53. The device of claim 51, wherein said electronic components comprise a plurality of independently addressable LED optical sources.

54. The device of claim 53, wherein at least one LED optical source has an emission output wavelength maximum that is at least 40 nm different from another LED optical source emission output wavelength maximum, thereby providing multiplex control of different color LED optical sources.

55. The device of any of claims 51-54, wherein said multi-channel antenna comprises: a plurality of capacitative coupling traces operably connected to said plurality of electronic components to provide non-overlapping resonance frequencies for selective energy harvesting and independent control of each of said plurality of independently addressable electronic components.

56. The device of any of claims 51-54, that is stretchable and flexible and capable of accommodating a strain greater than 10% without device failure.

57. The device of claim 56, wherein said substrate and superstrate comprise an elastomeric material having a Young's modulus of less than 10 MPa.
